(19) United States
(12) Patent Application Publication
(10) Pub. No.: US 2006/0286117 A1
(43) Pub. Date: Dec. 21, 2006

(54) Neuronally expressed stem cell factor modulates angiogenesis and neural stem cell migration to areas of brain injury

(76) Inventors: Howard A. Fine, Potomac, MD (US); Lixin Sun, Frederick, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/440,173

(22) Filed: May 24, 2006

Related U.S. Application Data
(63) Continuation-in-part of application No. PCT/US04/39376, filed on Nov. 22, 2004.
(60) Provisional application No. 60/525,760, filed on Nov. 26, 2003.
(60) Provisional application No. 60/563,397, filed on Apr. 19, 2004.

(51) Int. Cl. A61K 39/00; A61K 48/00

(52) U.S. Cl. 424/185.1; 514/44

(57) ABSTRACT

The present invention relates to methods of promoting or inhibiting angiogenesis in humans comprising administering to a human in need thereof an effective amount of Stem Cell Factor or a modulator of Stem Cell Factor or an agonist or antagonist of c-Kit and methods of inducing migration of a neural stem or progenitor cell to a site of neurological injury in the central nervous system of a subject.

### RELATED APPLICATIONS

This application is a continuation-in-part of International Patent Application No. PCT/US2004/039376 filed Nov. 22, 2004, designating the U.S. and published in English on Jun. 16, 2005 as WO 2005/053729, which claims the benefit of U.S. Provisional Application No. 60/525,760, filed Nov. 26, 2003, and U.S. Provisional Patent Application No. 60/563,397, filed Apr. 19, 2004, all of which are hereby incorporated by reference in their entireties.

### FIELD OF THE INVENTION

The invention is related to methods of modulating angiogenesis and methods of inducing migration of a neural stem or progenitor cell to a site of neurological injury in the central nervous system of a subject.

### BACKGROUND OF THE INVENTION

Part I. Angiogenesis

Malignant gliomas are among the most lethal tumors, with median survivals of less than a year for patients with the most common type of glioma, glioblastoma, despite aggressive surgery, radiation, and chemotherapy. In an attempt to identify novel therapeutic targets, many investigators have focused on the profound angiogenic response associated with malignant gliomas (Plate and Risau, 1995 Glia 15:339-347). Tumor-induced angiogenesis is one of the pathological hallmarks of malignant gliomas and has been demonstrated experimentally to be important for glioma progression. Thus, antiangiogenic strategies have great potential as a treatment approach for gliomas (Stratmann et al. 1997 Acta Neurochir Suppl (Wien) 68:105-110; Tanaka et al. 1998 Cancer Res 58:3362-3369 and Purow and Fine, 2004 Hematol Oncol Clin North Am 18:1161-1181). Identification of the most important angiogenic pathways utilized by gliomas is therefore of interest and significance.

It has been shown that many putative angiogenic factors are expressed by glioma cell lines in vitro; however, few of these factors (with the possible exception of vascular endothelial growth factor, VEGF), have been conclusively demonstrated to have any pathophysiological role in glioma progression in vivo (Goldbrunner et al. 2000 Neurosurgery 47:921-929; Dunn et al. 2000 J Neurooncol 50:121-137 and Lamszus et al. 2004 Cancer Treat Res 117:169-190). This is important, for it is known that tumor angiogenesis involves a complex interplay between tumor cells, endothelial cells (ECs), and other host cells as well as their surrounding extracellular matrix (host stroma) (Liotta et al. 1991 Cell 64:327-336; Carmeliet and Jain 2000 Nature 407:249-257; Tlsty and Hein, 2001 Curr Opin Genet Dev 11:54-59 and Kalluri, 2003 Nat Rev Cancer 3:422-433). Thus, a putative angiogenic factor may have very different functions in diverse tissue types and vascular beds. Given the unique microenvironment and microvascular architecture within the central nervous system (CNS), it would therefore not be surprising that various cytokines might have very different biological effects on the angiogenic process within the CNS compared to that in systemic capillary beds.

Stem cell factor (SCF) expression has been demonstrated in the past in a number of glioma cell lines, although its significance remains unclear. We and others have recently reported that various forms of brain injury induce neuronal expression of SCF, and that SCF mediates neural stem cell (NSC) migration to the site of cerebral injury (Zhang and Fedoroff 1999 Acta Neuropathol (Berl) 97:393-398; Jin et al. 2002 J Clin Invest 110:311-319; Sun et al. 2004 J Clin Invest 113:1364-1374 and Erlandsson et al. 2004 Exp Cell Res 301:201-210). Likewise, recent data demonstrate that hepatic injury induces SCF production and that SCF in turn promotes liver regeneration, an angiogenesis-dependent process (Ren et al. 2003 J Clin Invest 112:1407-1418 and Greene et al. 2003 Ann Surg 237:530-535). Consistent with a potential role in angiogenesis, the SCF receptor c-Kit is found on circulating endothelial precursor cells and human umbilical vein EC (HUVEC) (Broudy et al. 1994 Blood 83:2145-2152; Peichev et al. 2000 Blood 95:952-958 and Matsui et al. 2004 J Biol Chem 279:18600-18607).

Part II: Migration

Neural stem/progenitor cell (NSPC) migration is an essential process for the development of the central nervous system (CNS) as well as the ongoing neurogenesis that occurs in the mature CNS of most vertebrate species including mammals (Gage, F. H. 2002 J. Neurosci. 22:612-613; Alvarez-Buylla, A. and Lois, C. 1995 Stem Cells 13:263-272; Hatten, M. E. 1999 Annu. Rev. Neurosci. 22:511-539). It has been demonstrated that NSPC proliferate in the subventricular zone (SVZ) and migrate tangentially through the SVZ, in a pattern reminiscent of the rostral migratory stream (RMS), toward the olfactory bulb where they differentiate into mature neurons (Lois, C. et al. 1996 Science 271:978-981; Bedard, A. et al. 2002 Eur. J Neurosci. 16:1917-1924; Fukushima, N. et al. 2002 Neurosci. Res. 44:467-473). It has also been recently demonstrated that NSPC migrate to sites of pathological insult such as various types of brain injury (i.e., ischemic, blunt trauma) and tumors (Arvidsson, A. et al. 2002 Nat. Med. 8:963-970; Parent, J. M. et al. 2002 J. Neurosci. 22:3174-3188; Iwai, M. et al. 2003 J Cereb. Blood Flow Metab 23:331-341; Fricker, R. A. et al. 1999 J. Neurosci. 19:5990-6005; Aboody, K. S. et al. 2000 PNAS 97:12846-12851; Li, Z. et al. 2002 Neurosci. Res. 42:123-132). NSPC migration toward damaged CNS tissue may represent an adaptive response for the purpose of limiting and/or repairing damage although to date there are few data to definitively support or refute this hypothesis. Regardless of its physiological role in injury, the migratory properties of NSPC could theoretically be exploited for cell based therapeutics (Ehtesham, M. et al. 2002 Cancer Res. 62:7170-7174; Yip, S. et al. 2003 Cancer J. 9:189-204). Thus, the microenvironmental conditions and guidance signals that regulate NSPC migration in the adult need to be elucidated.

Although little is understood about the mechanism controlling NSPC migration in the adult CNS, several key mechanisms responsible for NSPC migration in embryonic development have been elucidated. Embryonic NSPC recognize cues provided by cells along the path of migration and are guided at long distances by gradients of chemoattractant molecules that are released selectively by cells along the way and at their final destination (Luskin, M. B. 1993 Neuron 11:173-189; Becker, P. S. et al. 1999 Exp. Hematol. 27:533-541; Song, H. & Poo, M. 2001 Nat. Cell Biol. 3:E81-E88). The discovery of netrins (Serafini, T. et al. 1994 Cell 78:409-424; Yee, K. T. et al. 1999 Neuron 24:607-622; Kennedy, T. E. et al. 1994 Cell 78:425-435; Meyerhardt, J. A. et al. 1999 Cell Growth Differ. 10:35-42), semaphorins (Spassky, N. et al. 2002 J. Neurosci. B5992-6004; Bagri, A. & Tessier-Lavigne, M. 2002 Adv. Exp. Med. Biol. 515:13-31; Kolodkin, A. L. et al. 1993 Cell 75:1389-1399), ephrins (Smith, A. et al. 1997 Curr. Biol. 7:561-570; Wang, H. U. & Anderson, D. J. 1997 Neuron 18:383-396), and Slit proteins (Rothberg, J. M. et al. 1990 Genes Dev. 4:2169-2187; Simpson, J. H. et al. 2000 Neuron 28:753-766; Wu, W. et al. 1999 Nature 400:331-336) established the existence of neuronal migration guidance signals at long distances by diffusible secreted proteins that have NSPC chemoattractant and repulsive properties. Recently, it has been suggested that certain cytokines with known important functions in CNS development (i.e., transforming growth factor alpha, bFGF, and EGF) were capable of enhancing ischemia-induced progenitor proliferation and migration (Nakatomi, H. et al. 2002 Cell 110:429-441; Forsberg-Nilsson, K. et al. 1998 J. Neurosci. Res. 53:521-530). Nevertheless, our understanding of the mechanisms guiding neural progenitor cell migration in the pathological states remains limited. Identification of diffusible NSPC chemoattractant factors would not only help to better understand the totality of mechanisms responsible for injury-mediated NSPC migration, but also could have significant therapeutic implications for the prospect of cell-based therapeutic strategies for injury repair.

### SEGUE TO THE INVENTION

In this study, we demonstrate that SCF is a potent angiogenic factor in vitro and in vivo. Furthermore, SCF expression is upregulated in gliomas in a grade-dependent manner, and tumor cells with the strongest SCF expression, as well as normal SCF-expressing neurons, are found predominantly within the infiltrating tumor border, an area that colocalizes with prominent angiogenesis. High SCF expression is associated with shorter patient survival, and downregulation of SCF expression in glioma cells suppresses glioma-induced angiogenesis and improves survival of the mice bearing intracranial glioma xenografts. Thus, the SCF/c-Kit pathway plays a prominent role in pathological angiogenesis within the CNS and provides a basis for use as a molecular target for glioma treatment.

In an attempt to identify NSPC chemoattractant molecules in injured brain tissue, we utilized subtractive cDNA suppression hybridization (SSH) to identify genes that were selectively upregulated during injury. We then utilized an in vitro NSPC migration assay to screen the products of our isolated cDNA clones for NSPC chemotactic activity. We now report the identification of stem cell factor (SCF) as a cytokine that is highly overexpressed by neurons at sites of brain injury. We additionally show that the SCF receptor (c-Kit) is expressed on NSPC and autophosphorylated following ligand binding and that SCF mediates potent chemoattractant activity for NSPC both in vitro and in vivo.

### SUMMARY OF THE INVENTION

The present invention relates to methods of promoting or inhibiting angiogenesis in humans comprising administering to a human in need thereof an effective amount of Stem Cell Factor or a modulator of Stem Cell Factor or an agonist or antagonist of c-Kit.

The invention also relates to methods of inducing migration of a neural stem or progenitor cell to a site of neurological injury in the central nervous system of a subject.

### BRIEF DESCRIPTION OF THE DRAWINGS

FIG. 1. Aberrant expression of SCF in gliomas. (A-C): Expression of the two splicing isoforms of SCF in human glioma cell lines. (A) sSCF and mSCF were determined by real-time PCR with isoform-specific probe sets, respectively. (B) Secreted SCF in cell conditioned media was measured using ELISA and normalized by total protein. (C) Western blot analysis of SCF expression. Tubulin was used as a loading control. Note that both SCF isoforms are expressed at higher levels by glioma cells than by normal astrocytes. (D and E): SCF mRNA is overexpressed in primary human high-grade glioma specimens. (D) cDNA microarray analysis of differential SCF expression in gliomas according to WHO grades. Note the positive correlation of SCF expression with increasing grade (grade III and IV gliomas compared to nontumor brain (NB); **p<0.001). (E) Increased SCF mRNA in high-grade gliomas was confirmed by real-time RT-PCR. Grade II oligodendroglioma (n=9) and astrocytoma (n=9); grade III, anaplastic oligodentrocytoma (n=8), anaplastic astrocytoma (n=9); grade IV (n=28); NB (n=9). (*p<0.05). Relative message indicates the ratio of SCF to β-actin. Columns and bars show the mean and SEM, respectively.

FIG. 2. SCF promotes angiogenesis in vitro. (A) c-Kit expression in ECs was determined by FACS in BMVEC-b, HUVEC, and HMVEC-d cells. The shaded histograms represent the fluorescence intensity of c-Kit, and the black line indicates the control IgG isotype staining. SK-N-SH and NIH3T3 cells were used as positive and negative controls, respectively (NIH3T3 not shown). (B) SCF alone sustained EC proliferation and survival as determined by thymidine incorporation. (C) HBMEC also showed dose-dependent growth in the presence of SCF. (D-F) SCF activates brain microvascular ECs. (D) In the presence of SCF, BMVEC-b migrated into the denuded zone 18 hr after scraping. Arrows indicate the wound edge. (E) Total cells in denuded zone were counted from three separate fields in each well from triplicates. (F) BMVEC-b formed tubular structures on Matrigel in the presence of SCF but not in controls. (G) The U251-derived CM significantly increased HUVEC proliferation in culture, an effect nearly completely blocked by neutralizing antibodies to either SCF or VEGF. Scale bar, 200 μM. Columns and bars show the mean and SEM, respectively.

FIG. 3. SCF/c-Kit activates MAPKs and Akt pathways in brain microvascular ECs. Cell lysates were collected at the indicated times after SCF stimulation of BMVEC-b and HUVEC (A) as well as HBMEC (B) and analyzed by Western blot with the indicated anti-phosphorylated protein antibodies as well as anti-total protein antibodies. Arrows: dark gray, phosphorylated protein; light gray, total protein. Tubulin was used as an equal loading control.

FIG. 4. A high density of blood vessels was induced in SCF- or β-FGF-supplemented Matrigel plugs. (A) H&E staining. Scale bars, 500 μM (LP), 50 μM (HP). (B) Immunofluorescence staining for vWF. Scale bars, 100 μM (LP), 50 μM (HP). (C) Western blot for Tie2 in homogenates of Matrigel plugs. Arrows: neovasculature containing red blood cells. LP, low power; HP, high power; M, muscle; S, skin; G, gel.

FIG. 5. Suppression of SCF in U373 glioma cells attenuates U373-mediated angiogenesis. (A) Downregulation of SCF in U373/as-SCF, U87/as-SCF, and U251/shRNA SCF was verified by Western blot. Downregulation of SCF resulted in no effects on VEGF expression from the cells. (B and C): Immunofluorescence staining for vWF (B) and H&E staining (C) in an in vivo tumor-angiogenesis assay using U373/as-SCF and U373/vector glioma cells. Scale bars, 100 μM (LP in B), 500 μM (LP in C), 50 μM (HP). (D) Tie2 protein in homogenates of Matrigel plugs (40 mg total protein in each sample) was immunoprecipitated with Tie2 antibody and then detected by Western blot in duplicate. The comparative quantitative intensities of the amount of Tie2 protein were measured by densitometry. Arrows: neovasculature containing red blood cells. LP, low power; HP, high power; M, muscle; S, skin; G, gel. Columns and bars show the mean and SEM, respectively.

FIG. 6. Downregulation of SCF expression in glioma cells impedes tumor angiogenesis and prolongs survival in an intracranial tumor model. (A) Kaplan-Meier survival curve shows that downregulation of SCF expression in U373/as-SCF, U87/as-SCF, and U251/shRNA SCF tumors significantly improves survival of tumor-bearing mice compared with their vector controls, respectively (log-rank test p<0.05). (B) Downregulation of SCF expression attenuates SCF-induced angiogenesis in implanted glioma tumors. In contrast to U373/vector gliomas, the U373/as-SCF gliomas demonstrate little SCF staining and a markedly lower density of blood vessels. Scale bars, 200 μM (LP); 50 μM (HP). (C) Colocalization of neovasculature with SCF in U373/vector tumors. Tumor tissue shows strong SCF expression and a high density of collapsed vessels (upper panels). Along the tumor invasion border (middle panels), abundant SCF surrounds tumor cluster with multiple small vessels in the center. In brain tissue adjacent to the tumor mass (lower panels), SCF is expressed both by tumor cells and normal cells within the brain. Scale bar, 50 μM. (D and E): Intracranial tumor induces SCF expression in affected neurons. (D) SCF-positive neurons (arrows) are seen in the cerebral cortex infiltrated by tumor (T) as detected by in situ hybridization using a SCF antisense probe (left). The negative control using a hybridized sense probe is seen on the right. (E) SCF-expressing cells are present in the cerebral cortex close to U373/as-SCF injection (left) as compared to the contralateral side of the brain (right) as analyzed by immunohistochemistry. Arrows: dark gray, SCF staining; light gray, vWF+blood vessels. Scale bars, 50 μM.

FIG. 7. Distribution of SCF expression in human brain harboring a glioblastoma. (A-D): SCF-expressing cells by immunohistochemistry in representative regions of brain and tumor. (A) Center of tumor mass. (B) Cerebral white matter infiltrated by tumor cells. (C) Cerebral cortex with infiltration of tumor cells. (D) Cellular tumor adjacent to white matter. Scale bars (shown in D), 100 μM (LP), 50 μM (HP). (E) SCF-expressing tumor cells surrounding new blood vessels are shown at higher magnification (800×). The top panels show the SCF-expressing tumor cells (left) and SCF-negative cells (right) lining blood vessels, respectively. The middle and bottom panels show SCF-expressing cells lining the full-length of the blood vessels. Arrowheads indicate cytoplasmic staining of SCF in tumor cells (black), neuron (gray), and the boundary between tumor mass (TM) and tumor-infiltrating white matter (WM) (white). Note that SCF-expressing tumor cells are more pronounced in the tumor regions adjacent to normal brain and along the new blood vessels. Scale bars, 25 μM. (F) c-Kit-positive endothelial cells on the glioblastoma-associated vasculature colocalized with CD31-positive endothelial cells (left panels) throughout the tumor, including in areas adjacent to necrosis (N, right panel). Scale bars, 50 μM (left panels), 100 μM (right panel). (G) Kaplan-Meier survival curve demonstrating survival of patients with glioblastoma (grade IV gliomas) stratified by tumors expressing either low or high levels of SCF (log-rank analyses p=0.0004) and expressing low or high levels of VEGF (log-rank analyses p=0.2458).

FIG. 8. Expression of c-Kit on glioma cells by FACS analysis. The shaded histograms represent the fluorescence intensity of c-Kit and the black line indicates the control IgG isotype staining. SK-N-SH cells were used as positive control. A) Expression of c-Kit on the cell membrane. B) Total c-Kit expression on the cell membrane and in the intracytoplasm. Note that the majority of c-Kit is mainly expressed within the cytosol, not on the cell membrane. M1 markers have been set at the 98% confidence limit. Y-axis: cell counts; X-axis: c-Kit intensity.

FIG. 9. Inhibition of either SCF (by siRNA expression) or VEGF activity (through systemic administration of a VEGF antibody; bevacizumab) suppressed subcutaneously implanted human glioma growth in nude mice.

FIG. 10. SCF induced by “freeze” injury to the brain. (A) Schematic illustration of “freeze” injury. Arrow indicates the insertion track of the pre-cooled needle. Lines separate the forebrain into injured and uninjured hemispheres and divide the hemispheres into its dorsal and ventral halves. CX, cortex; CPu, caudate putamen (striatum); CC, corpus callosum; LV, lateral ventricle. (B) Custom microarray confirms SCF message increased in the injured brain at day 5. The cDNA clones from SSH library were loaded individually on to duplicate nitrocellulose filters, one hybridized by the injured brain-derived cDNAs (Injury), and another one hybridized by contralateral control (Control). Membranes with the SCF gene are shown, and the SCF spots are marked with circles in the middle rows of the microarrays. Actin and GAPDH, are shown as circles in the upper and lower rows of the microarrays. SCF images are stronger in the injured brain-derived cDNA-hybridized blot than the control blot. (C) Quantitative RT-PCR. Fold induction of SCF mRNA in “freeze”-injured forebrain 5 days after injury compared to the uninjured contralateral side (P<0.05). The SCF mRNAs were individually normalized to levels of 18S RNA. (D) Time-dependent changes of SCF protein induction after “freeze” brain injury as measured by Western blot, using uninjured forebrain as a control. Two major bands were present, representing the transmembrane form of SCF (33 kDa) and the cleaved, soluble form of SCF (19 kDa). (E) Quantitation of SCF expression by computer densitometry. The band intensities of SCF were normalized to those of β-tubulin.

FIG. 11. Distribution of SCF in the injured forebrain. (A-G) Sections were immunostained with an Ab against SCF and visualized with DAB. Counterstaining was done with hematoxylin. (A) In the normal cortex, SCF positive cells exist mainly in the surface of the cortex, in layers I and II. (B) In the “freeze”-injured forebrain, the distribution of SCF-staining cells include the whole depth of cortex, with intensively SCF-positive cells in the layers III, IV. (C and D): Magnified images of the black boxes in A and B, respectively. Abundant SCF-staining cells were observed in the injured area indicated by arrows (D). (E and F): SCF expression was also present in the SVZ of injured brain (F), but was not detectable in the control SVZ (E). (G) Large numbers of SCF-staining cells were present around the injury (boxes I and II), while the SCF-positive cells decreased with increasing distance away from the site of injury (boxes III and IV). Bottom row, higher-magnification views (×200) of each section as indicated by the arrows. Boxes I-IV are schematic representation of the areas used to quantify cell numbers. Scale bar (shown in A): A and B, 200 μm; C-F, 50 μm; G, main image, 250 μm, and higher magnification, 70 μm. CC, corpus callosum; LV, lateral ventricle; Str, striatum. (H) Quantitation of SCF-positive cells in boxes I-IV of G. The SCF-positive cells are presented as a percentage of the total cell number for each section. Comparisons are corrected for surface area and total cells in the section. Statistical differences were determined by comparison of boxes III and IV with box I (*p<0.05).

FIG. 12. Characterization of SCF-positive cells in the cortex at 7 days after injury. (A-H): Sections were double immunostained for TUJ-1 and SCF (A and B), MAP2 and SCF (C and D), GFAP and SCF (E and F), or lectin RCA I and SCF (G and H). White boxes on panels A, C, E and G are magnified in panel B, D, F, and H, respectively. The enlarged images show the double staining for TUJ-1 and SCF or MAP2 and SCF (arrows), GFAP, RCA I or SCF single-positive cells (arrowheads). Scale bar (shown in A): A, C, E and G, 50 μm; B, D, and H, 16 μm; F, 18 μm.

FIG. 13. Expression of c-Kit on NSPCs in vitro and in vivo. (A) Expression of c-Kit mRNA in human NSPCs (hNSPCs) and mouse NSPCs (mNSPCs) as measured by semi-quantitative RT-PCR. (B and C): Immunofluorescence analysis for c-Kit and nestin in mouse (B) and human (C) NSPC. (D and E): Expression of c-Kit on NSPCs in vivo. (D) Double staining of mouse brain by c-Kit and nestin revealed c-Kit expression in nestin-positive cells (arrow) in the SVZ. (E) Representative orthogonal image of colocalization of c-Kit and nestin in vivo. Upper, main, and left panel shows the views of xz, xy, and yz plane, respectively. Lines represent coordinates in each plane. x axis, 24.4 μm; y axis, 24.4 μm; z axis, 6.7 μm; optical section thickness, 0.02 μm. Scale bar: B-D, 20 μm (shown in B).

FIG. 14. SCF/c-Kit pathway is involved in injury-induced NSPC migration. (A) SCF-induced c-Kit tyrosine phosphorylation (Tyr-P) was detected by immunoprecipitation (IP) and immunoblot (IB). The 120-kDa and 140-kDa bands represent the c-Kit proteins in the human and mouse NSPCs. Lane 1, SCF treatment; lane 2, untreated control; lane 3, pretreatment of NSPCs with ACK45 c-Kit-blocking Ab before SCF treatment. (B) Tissue lysates from injured and normal mouse forebrain were used to stimulate migration of mouse NSPCs (with or without pretreatment of ACK45 blocking Ab) in the Boyden chamber migration assay. Relative fluorescence unit (RFU) correlated with the number of migrated cells. NSPCs migration was significantly induced by injured brain lysates compared to normal brain lysates (*P<0.05). The chemotactic effect of injured brain lysates was nearly completely abolished when NSPC were pretreated with the c-Kit-blocking Ab (*P<0.05). Error bars represent SEM. These are representative experiments and similar results were obtained from at least three independent experiments.

FIG. 15. Effects of rmSCF on NSPC migration in the Boyden chamber migration assay. Mouse and human NSPC migration was stimulated in a dose-dependent manner by soluble rmSCF. Error bars represent SEM. *P<0.05, **P<0.001, respectively. n=3. This is a representative experiment, similar results were obtained from three independent experiments.

FIG. 16. SCF-stimulated progenitor cell migration in vivo. (A) Quantitation of BrdU-labeled cells in the injected areas (1.5 mm²) and in the contralateral side of the brain. Significantly more BrdU labeled cells were seen in the SCF-injected area than in the PBS injected area or in the contralateral side of the SCF-injected brain (P<0.001, n=6). Inj. injection side; contra. contralateral side to SCF injection. (B) Representative images of BrdU staining in the cortical areas. (c) Schematic diagram of the brain used to examine the response of NPSC to rmSCF is shown. The arrow indicates the intracerebral injection track (as described in Example 2). (D) Normal distribution of BrdU-positive cells. These cells were detected primarily in the SVZ prior to SCF injection and were also positive for phospho-histone H3 staining (p-H3). (E and F): Immunohistochemistry of SCF injected brain with BrdU and phospho-histone H3 antibodies in the LVZ (E) and SCF injected cortex (F). (G) Nestin expression of BrdU positive cells in the SVZ. (H) Three-dimensional digital image of the cells indicated by the arrows in (G) is shown. Upper, main, and right panel shows the view of xz, xy, and yz plane, respectively. Lines represent coordinates in each plane. x axis, 23.2 μm; y axis, 23.2 μm; z axis, 11 μm; optical section thickness: 1.1 μm. Scale bar (shown in B): B, 100 μm; D, 64 μm; E and F, 32 μm; G, 12 μm . Abbreviation: CC, corpus callosum; LV, Lateral ventricle; Str striatum.

FIG. 17. SCF stimulated progenitor cell migration in vivo. (A and B): Whole-brain image of DiI labeling after ventricular injection of DiI. DiI-labeled cells were only detected in the lining of ventricular zone. Boxed regions in (A) are shown with higher magnification in (B). DiI staining was confined to the nuclei and process of cells. (C and D): Representative images of control hemisphere (C) and SCF-injected hemisphere (D). Note the greater number of DiI positive cells in the SCF injection side. (E) Whole-brain image of GFP-positive cells in the brain. (F and G): Representative images of control hemisphere (F) and SCF-injected hemisphere (G). Scale bar (shown in B): A and E, 100 μm; B-D, F and G, 20 μm.

FIG. 18. SCF stimulates angiogenesis in vivo. Gel: Plugged matrigel; M: muscle; RBC: red blood cell.

FIG. 19. SCF stimulated DNA synthesis of bMVEC-B cell.

FIG. 20. SCF enhanced bMVEC-B cell tube formation on matrigel.

### BRIEF DESCRIPTION OF THE SEQUENCES

Stem Cell Factor (SCF)

Nucleotide and predicted amino acid sequence of human SCF isoform KL-1 are given by GenBank Accession No. NM_{—}000899, having 273 amino acids total, a signal peptide of 25 amino acids so that the 248 amino acid transmembrane form extends from GenBank Accession No. NM_{—}000899 amino acid #26-273 and a proteolytic cleavage site in the extracellular domain at Ala 165 (counting from the beginning of the mature protein) so that the soluble form extends 165 amino acids from GenBank Accession No. NM_{—}000899 amino acid #26-190. Nucleotide and predicted amino acid sequence of human SCF isoform KL-2 are given by GenBank Accession No. NM_{—}003994, which is an alternatively spliced and membrane bound form having 245 amino acids total.

c-Kit

Nucleotide and predicted amino acid sequence of human c-Kit proto-oncogene are given by GenBank Accession No. X06182, having 976 amino acids.

### DETAILED DESCRIPTION OF THE PREFERRED EMBODIMENT

### DEFINITIONS

Unless defined otherwise, technical and scientific terms used herein have the same meaning as commonly understood by one of ordinary skill in the art to which this invention belongs. See, e.g., Dorland's illustrated medical dictionary (30^{th} Edition), D. M. Anderson, P. D. Novak, J. Keith and M. A. Elliott, Eds. Saunders (an Imprint of Elsevier), Philadelphia, Pa., 2003.

The term “agonist” refers to a drug that has affinity for and stimulates physiologic activity at a cell receptor normally stimulated by naturally occurring substances.

The term “antagonist” refers to a substance that tends to nullify the action of another as a drug that binds to a cell receptor without eliciting a biological response, blocking binding of substances that could elicit such responses.

The term “angiogenesis” refers to any formation of new blood vessels.

The term “ECD” means “extracellular domain”.

The term “TM” means “transmembrane domain”.

The term “CD” means “cytoplasmic domain”.

The term “active” refers to those forms of the polypeptide that retain the biologic and/or immunologic activities of any naturally occurring polypeptide. According to the invention, the terms “biologically active” or “biological activity” refer to a protein or peptide having structural, regulatory or biochemical functions of a naturally occurring molecule. The term “SCF-like” refers to biological activity that is similar to the biological activity of a stem cell factor. Likewise “biologically active” or “biological activity” refers to the capability of the natural, recombinant or synthetic SCF-like peptide, or any peptide thereof, to induce a specific biological response in appropriate animals or cells and to bind with specific antibodies.

The term “(c-Kit)-like” refers to biological activity that is similar to the biological activity of c-Kit. Likewise “biologically active” or “biological activity” refers to the capability of the natural, recombinant or synthetic (c-Kit)-like peptide, or any peptide thereof, to induce a specific biological response in appropriate animals or cells and to bind with specific antibodies.

The terms “complementary” or “complementarity” refer to the natural binding of polynucleotides by base pairing. For example, the sequence 5′-AGT-3′ binds to the complementary sequence 3′-TCA-5′. Complementarity between two single-stranded molecules may be “partial” such that only some of the nucleic acids bind or it may be “complete” such that total complementarity exists between the single stranded molecules. The degree of complementarity between the nucleic acid strands has significant effects on the efficiency and strength of the hybridization between the nucleic acid strands.

According to terminology adopted from hematopoiesis, neural precursor cells are often classified as stem cells (defined as self-renewing, totipotent precursors), progenitor cells (which arise from stem cells and are not self-renewing, but which can give rise to multiple cell types such as neurons and astrocytes), and committed precursors (which give rise to only a single cell type, such as neurons, but are not yet functionally mature) (Gage 2000 Science 287:1433-1438). In this application, however, the terms stem cell, progenitor and precursor will be used interchangeably unless stated otherwise.

The term “expression modulating fragment”, EMF, means a series of nucleotides that modulates the expression of an operably linked ORF or another EMF.

As used herein, a sequence is said to “modulate the expression of an operably linked sequence” when the expression of the sequence is altered by the presence of the EMF. EMFs include, but are not limited to, promoters, and promoter modulating sequences (inducible elements). One class of EMFs is nucleic acid fragments that induce the expression of an operably linked ORF in response to a specific regulatory factor or physiological event.

The terms “nucleotide sequence” or “nucleic acid” or “polynucleotide” or “oligonucleotide” are used interchangeably and refer to a heteropolymer of nucleotides or the sequence of these nucleotides. These phrases also refer to DNA or RNA of genomic or synthetic origin that may be single-stranded or double-stranded and may represent the sense or the antisense strand, to peptide nucleic acid (PNA) or to any DNA-like or RNA-like material. In the sequences, A is adenine, C is cytosine, G is guanine and T is thymine while N is A, C, G, or T (U). It is contemplated that where the polynucleotide is RNA, the T (thymine) in the sequence may be replaced with U (uracil). Generally, nucleic acid segments provided by this invention may be assembled from fragments of the genome and short oligonucleotide linkers, or from a series of oligonucleotides, or from individual nucleotides, to provide a synthetic nucleic acid that is capable of being expressed in a recombinant transcriptional unit comprising regulatory elements derived from a microbial or viral operon, or a eukaryotic gene.

The terms “oligonucleotide fragment” or a “polynucleotide fragment”, “portion”, or “segment” or “probe” or “primer” are used interchangeably and refer to a sequence of nucleotide residues that are at least about 5 nucleotides, more preferably at least about 7 nucleotides, more preferably at least about 9 nucleotides, more preferably at least about 11 nucleotides and most preferably at least about 17 nucleotides. The fragment is preferably less than about 500 nucleotides, preferably less than about 200 nucleotides, more preferably less than about 100 nucleotides, more preferably less than about 50 nucleotides and most preferably less than 30 nucleotides. Preferably the probe is from about 6 nucleotides to about 200 nucleotides, preferably from about 15 to about 50 nucleotides, more preferably from about 17 to 30 nucleotides and most preferably from about 20 to 25 nucleotides. Preferably the fragments can be used in polymerase chain reaction (PCR), various hybridization procedures or microarray procedures to identify or amplify identical or related parts of mRNA or DNA molecules. A fragment or segment may uniquely identify each polynucleotide sequence of the present invention.

Probes may, for example, be used to determine whether specific mRNA molecules are present in a cell or tissue or to isolate similar nucleic acid sequences from chromosomal DNA as described by Walsh et al. (Walsh, P. S. et al. 1992 PCR Methods Appl 1:241-250). They may be labeled by nick translation, Klenow fill-in reaction, PCR, or other methods well known in the art. Probes of the present invention, their preparation and/or labeling are elaborated in Sambrook J et al. 1989 Molecular Cloning: A Laboratory Manual, Cold Spring Harbor Laboratory, NY; or Ausubel, et al. (eds.) 1992 Current Protocols in Molecular Biology, John Wiley & Sons.

The term “open reading frame”, ORF, means a series of nucleotide triplets coding for amino acids without any termination codons and is a sequence translatable into protein.

The terms “operably linked” or “operably associated” refer to functionally related nucleic acid sequences. For example, a promoter is operably associated or operably linked with a coding sequence if the promoter controls the transcription of the coding sequence. While operably linked nucleic acid sequences can be contiguous and in the same reading frame, certain genetic elements, e.g., repressor genes are not contiguously linked to the coding sequence but still control transcription/translation of the coding sequence.

The terms “polypeptide” or “peptide” or “amino acid sequence” refer to an oligopeptide, peptide, polypeptide or protein sequence or fragment thereof and to naturally occurring or synthetic molecules. A polypeptide “fragment”, “portion”, or “segment” is a stretch of amino acid residues of at least about 5 amino acids, preferably at least about 7 amino acids, more preferably at least about 9 amino acids and most preferably at least about 17 or more amino acids. The peptide preferably is not greater than about 200 amino acids, more preferably less than 150 amino acids and most preferably less than 100 amino acids. Preferably the peptide is from about 5 to about 200 amino acids. To be active, any polypeptide must have sufficient length to display biological and/or immunological activity.

The term “naturally occurring polypeptide” refers to polypeptides produced by cells that have not been genetically engineered and specifically contemplates various polypeptides arising from post-translational modifications of the polypeptide including, but not limited to, acetylation, carboxylation, glycosylation, phosphorylation, lipidation and acylation.

The term “translated protein coding portion” means a sequence that encodes for the full length protein that may include any leader sequence or a processing sequence.

The term, “mature protein coding sequence” refers to a sequence that encodes a peptide or protein without any leader/signal sequence. The “mature protein portion” refers to that portion of the protein without the leader/signal sequence. The peptide may have the leader sequences removed during processing in the cell or the protein may have been produced synthetically or using a polynucleotide only encoding for the mature protein coding sequence. It is contemplated that the mature protein portion may or may not include an initial methionine residue. The initial methionine is often removed during processing of the peptide.

The term “derivative” refers to polypeptides chemically modified by such techniques as ubiquitination, labeling (e.g., with radionuclides or various enzymes), covalent polymer attachment such as pegylation (derivatization with polyethylene glycol) and insertion or substitution by chemical synthesis of amino acids such as ornithine, which do not normally occur in human proteins.

The term “variant” (or “analog”) refers to any polypeptide differing from naturally occurring polypeptides by amino acid insertions, deletions, and substitutions, created using, e.g., recombinant DNA techniques. Guidance in determining which amino acid residues may be replaced, added or deleted without abolishing activities of interest, may be found by comparing the sequence of the particular polypeptide with that of homologous peptides and minimizing the number of amino acid sequence changes made in regions of high homology (conserved regions) or by replacing amino acids with consensus sequence.

Alternatively, recombinant variants encoding these same or similar polypeptides may be synthesized or selected by making use of the “redundancy” in the genetic code. Various codon substitutions, such as the silent changes that produce various restriction sites, may be introduced to optimize cloning into a plasmid or viral vector or expression in a particular prokaryotic or eukaryotic system. Mutations in the polynucleotide sequence may be reflected in the polypeptide or domains of other peptides added to the polypeptide to modify the properties of any part of the polypeptide, to change characteristics such as ligand-binding affinities, interchain affinities, or degradation/turnover rate.

Preferably, amino acid “substitutions” are the result of replacing one amino acid with another amino acid having similar structural and/or chemical properties, i.e., conservative amino acid replacements. “Conservative” amino acid substitutions may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. “Insertions” or “deletions” are preferably in the range of about 1 to 20 amino acids, more preferably 1 to 10 amino acids. The variation allowed may be experimentally determined by systematically making insertions, deletions, or substitutions of amino acids in a polypeptide molecule using recombinant DNA techniques and assaying the resulting recombinant variants for activity.

Alternatively, where alteration of function is desired, insertions, deletions or non-conservative alterations can be engineered to produce altered polypeptides. Such alterations can, for example, alter one or more of the biological functions or biochemical characteristics of the polypeptides of the invention. For example, such alterations may change polypeptide characteristics such as ligand-binding affinities, interchain affinities, or degradation/tunover rate. Further, such alterations can be selected so as to generate polypeptides that are better suited for expression scale up and the like in the host cells chosen for expression. For example, cysteine residues can be deleted or substituted with another amino acid residue in order to eliminate disulfide bridges.

The terms “purified” or “substantially purified” as used herein denotes that the indicated nucleic acid or polypeptide is present in the substantial absence of other biological macromolecules, e.g., polynucleotides, proteins, and the like. In one embodiment, the polynucleotide or polypeptide is purified such that it constitutes at least 95% by weight, more preferably at least 99% by weight, of the indicated biological macromolecules present (but water, buffers, and other small molecules, especially molecules having a molecular weight of less than 1000 daltons, can be present).

The term “isolated” as used herein refers to a nucleic acid or polypeptide separated from at least one other component (e.g., nucleic acid or polypeptide) present with the nucleic acid or polypeptide in its natural source. In one embodiment, the nucleic acid or polypeptide is found in the presence of (if anything) only a solvent, buffer, ion, or other components normally present in a solution of the same. The terms “isolated” and “purified” do not encompass nucleic acids or polypeptides present in their natural source.

The term “recombinant,” when used herein to refer to a polypeptide or protein, means that a polypeptide or protein is derived from recombinant (e.g., microbial, insect, or mammalian) expression systems. “Microbial” refers to recombinant polypeptides or proteins made in bacterial or fungal (e.g., yeast) expression systems. As a product, “recombinant microbial” defines a polypeptide or protein essentially free of native endogenous substances and unaccompanied by associated native glycosylation. Polypeptides or proteins expressed in most bacterial cultures, e.g., E. coli, will be free of glycosylation modifications; polypeptides or proteins expressed in yeast will have a glycosylation pattern in general different from those expressed in mammalian cells.

The term “recombinant expression vehicle or vector” refers to a plasmid or phage or virus or vector, for expressing a polypeptide from a DNA (RNA) sequence. An expression vehicle can comprise a transcriptional unit comprising an assembly of (1) a genetic element or elements having a regulatory role in gene expression, for example, promoters or enhancers, (2) a structural or coding sequence that is transcribed into mRNA and translated into protein, and (3) appropriate transcription initiation and termination sequences. Structural units intended for use in yeast or eukaryotic expression systems preferably include a leader sequence enabling extracellular secretion of translated protein by a host cell. Alternatively, where recombinant protein is expressed without a leader or transport sequence, it may include an amino terminal methionine residue. This residue may or may not be subsequently cleaved from the expressed recombinant protein to provide a final product.

The term “recombinant expression system” means host cells that have stably integrated a recombinant transcriptional unit into chromosomal DNA or carry the recombinant transcriptional unit extrachromosomally. Recombinant expression systems as defined herein will express heterologous polypeptides or proteins upon induction of the regulatory elements linked to the DNA segment or synthetic gene to be expressed. This term also means host cells that have stably integrated a recombinant genetic element or elements having a regulatory role in gene expression, for example, promoters or enhancers. Recombinant expression systems as defined herein will express polypeptides or proteins endogenous to the cell upon induction of the regulatory elements linked to the endogenous DNA segment or gene to be expressed. The cells can be prokaryotic or eukaryotic.

The term “secreted” includes a protein that is transported across or through a membrane, including transport as a result of signal sequences in its amino acid sequence when it is expressed in a suitable host cell. “Secreted” proteins include without limitation proteins secreted wholly (e.g., soluble proteins) or partially (e.g., receptors) from the cell in which they are expressed. “Secreted” proteins also include without limitation proteins that are transported across the membrane of the endoplasmic reticulum. “Secreted” proteins are also intended to include proteins containing non-typical signal sequences (e.g., Interleukin-1 Beta, see Krasney, P. A. & Young, P. R. 1992 Cytokine 4:134-143) and factors released from damaged cells (e.g., Interleukin-1 Receptor Antagonist, see Arend, W. P. et al. 1998 Annu Rev Immunol 16:27-55).

Where desired, an expression vector may be designed to contain a “signal or leader sequence” that will direct the polypeptide through the membrane of a cell. Such a sequence may be naturally present on the polypeptides of the present invention or provided from heterologous protein sources by recombinant DNA techniques.

The term “stringent” is used to refer to conditions that are commonly understood in the art as stringent. Stringent conditions can include highly stringent conditions (i.e., hybridization to filter-bound DNA in 0.5 M NaHPO₄, 7% sodium dodecyl sulfate (SDS), 1 mM EDTA at 65° C., and washing in 0.1×SSC/0.1% SDS at 68° C.), and moderately stringent conditions (i.e., washing in 0.2×SSC/0.1% SDS at 42° C.). Other exemplary hybridization conditions are described herein in the examples.

In instances of hybridization of deoxyoligonucleotides, additional exemplary stringent hybridization conditions include washing in 6×SSC/0.05% sodium pyrophosphate at 37° C. (for 14-base oligonucleotides), 48° C. (for 17-base oligonucleotides), 55° C. (for 20-base oligonucleotides), and 60° C. (for 23-base oligonucleotides).

As used herein, “substantially equivalent” or “substantially similar” can refer both to nucleotide and amino acid sequences, for example a mutant sequence, that varies from a reference sequence by one or more substitutions, deletions, or additions, the net effect of which does not result in an adverse functional dissimilarity between the reference and subject sequences. Typically, such a substantially equivalent sequence varies from one of those listed herein by no more than about 35% (i.e., the number of individual residue substitutions, additions, and/or deletions in a substantially equivalent sequence, as compared to the corresponding reference sequence, divided by the total number of residues in the substantially equivalent sequence is about 0.35 or less). Such a sequence is said to have 65% sequence identity to the listed sequence. In one embodiment, a substantially equivalent, e.g., mutant, sequence of the invention varies from a listed sequence by no more than 30% (70% sequence identity); in a variation of this embodiment, by no more than 25% (75% sequence identity); and in a further variation of this embodiment, by no more than 20% (80% sequence identity) and in a further variation of this embodiment, by no more than 10% (90% sequence identity) and in a further variation of this embodiment, by no more that 5% (95% sequence identity). Substantially equivalent, e.g., mutant, amino acid sequences according to the invention preferably have at least 80% sequence identity with a listed amino acid sequence, more preferably at least 85% sequence identity, more preferably at least 90% sequence identity, more preferably at least 95% sequence identity, more preferably at least 98% sequence identity, and most preferably at least 99% sequence identity. Substantially equivalent nucleotide sequence of the invention can have lower percent sequence identities, taking into account, for example, the redundancy or degeneracy of the genetic code. Preferably, the nucleotide sequence has at least about 65% identity, more preferably at least about 75% identity, more preferably at least about 80% sequence identity, more preferably at least 85% sequence identity, more preferably at least 90% sequence identity, more preferably at least about 95% sequence identity, more preferably at least 98% sequence identity, and most preferably at least 99% sequence identity. For the purposes of the present invention, sequences having substantially equivalent biological activity and substantially equivalent expression characteristics are considered substantially equivalent. For the purposes of determining equivalence, truncation of the mature sequence (e.g., via a mutation that creates a spurious stop codon) should be disregarded. Sequence identity may be determined, e.g., using the Jotun Hein method (Hein, J. 1990 Methods Enzymol. 183:626-645). Identity between sequences can also be determined by other methods known in the art, e.g., by varying hybridization conditions.

The term “transformation” means introducing DNA into a suitable host cell so that the DNA is replicable, either as an extrachromosomal element, or by chromosomal integration. The term “transfection” refers to the taking up of an expression vector by a suitable host cell, whether or not any coding sequences are in fact expressed. The term “infection” refers to the introduction of nucleic acids into a suitable host cell by use of a virus or viral vector.

Each of the above terms is meant to encompass all that is described for each, unless the context dictates otherwise.

Angiogenesis

Angiogenesis is the process by which new-blood vessels are formed (Folkman et al. 1992 J Biol Chem 267:10931-10934). Thus, angiogenesis is essential in reproduction, development, and wound repair. However, inappropriate angiogenesis can have severe consequences. For example, it is only after many solid tumors are vascularized as a result of angiogenesis that the tumors begin to grow rapidly and metastasize. Because angiogenesis is so critical to these functions, it must be carefully regulated, in order to maintain health. The angiogenesis process is believed to begin with the degradation of the basement membrane by proteases secreted from endothelial cells (EC) activated by mitogens such as vascular endothelial growth factor (VEGF) and basic fibroblast growth factor (bFGF). The cells migrate and proliferate, leading to the formation of solid endothelial cell sprouts into the stromal space, then, vascular loops are formed and capillary tubes develop with formation of tight junctions and deposition of new basement membrane.

The rate of angiogenesis involves a change in the local equilibrium between positive and negative regulators of the growth of microvessels. Abnormal angiogenesis occurs when the body loses its control of angiogenesis, resulting in either excessive or insufficient blood vessel growth. For instance, conditions such as myocardial infarction, peripheral vascular occlusion, brain ischemia, or stroke may result from the absence of angiogenesis normally required for natural healing. On the contrary, excessive blood vessel proliferation may favor tumor growth and spreading, blindness, psoriasis and rheumatoid arthritis.

The feasibility of gene therapy for modulating angiogenesis has been demonstrated. For example, promoting angiogenesis in the treatment of ischemia was demonstrated in a rabbit model and in human clinical trials with VEGF using a Hydrogel-coated angioplasty balloon as the gene delivery system. Successful transfer and sustained expression of the VEGF gene in the vessel wall subsequently augmented neovascularization in the ischemic limb (Takeshita et al. 1996 Laboratory Investigation 75:487-502; Isner, J. M. et al. 1996 Lancet 348:370-374).

Alternative methods for regulating angiogenesis are still desirable for a number of reasons. For example, it is believed that native endothelial cell (EC) number and/or viability decreases over time. Thus, in certain patient populations, e.g., the elderly, the resident population of ECs that is competent to respond to administered angiogenic cytokines may be limited. Moreover, while agents promoting or inhibiting angiogenesis may be useful at one location, they may be undesirable at another location. Thus, means to more precisely regulate angiogenesis at a given location are desirable.

Methods of Inducing Angiogenesis

In one embodiment, the present invention provides methods of promoting angiogenesis. As used herein, the term “SCF activity” refers to the activities associated with SCF. Accordingly, SCF or any other agent that increases the expression of SCF or promotes the functional activity of SCF, or agonists of c-Kit, can be used to induce angiogenesis. Examples of diseases and conditions that can be treated by these molecules include myocardial infarction, peripheral vascular occlusion, brain ischemia, and stroke.

Methods of Preventing Angiogenesis

In another embodiment, the present invention provides methods of inhibiting angiogenesis using agents that inhibit the expression of SCF or c-Kit. Examples of inhibitors of SCF expression are antisense molecules, ribozymes, and short interfering RNAs. In one embodiment, soluble c-Kit protein can be administered to bind and inactivate SCF. An agent that inhibits the expression of SCF or c-Kit is useful to prevent diseases or conditions such as restenosis, vascular bypass graft occlusion, transplant coronary vasculopathy, rheumatoid arthritis, psoriasis, ocular neovascularization, diabetic retinopathy, neovascular glaucoma, angiogenesis-dependent tumors and tumor metastasis.

Agents that inhibit the functions of SCF or c-Kit are also useful in inhibiting angiogenesis, especially in cancerous cells to prevent metastases. Examples of such agents include, but are not limited to, SCF or c-Kit antibodies and inhibitors of SCF or c-Kit function.

Nucleic Acids of the Invention

The invention is based on the discovery of a SCF or SCF-like polypeptide, the polynucleotides encoding the SCF or SCF-like polypeptide and the use of these compositions for modulating angiogenesis and for the diagnosis, treatment or prevention of neurological conditions and disorders.

The isolated polynucleotides of the invention include, but are not limited to a polynucleotide comprising any of the nucleotide sequences of GenBank Accession No. NM_{—}000899; a fragment of GenBank Accession No. NM_{—}000899; a polynucleotide of GenBank Accession No. NM_{—}000899 encoding the full length protein; a polynucleotide of GenBank Accession No. NM_{—}000899 encoding the mature protein; and a polynucleotide of GenBank Accession No. NM_{—}000899 encoding the soluble form of the protein. The polynucleotides of the present invention also include, but are not limited to, a polynucleotide that hybridizes under stringent conditions to (a) the complement of any of the polynucleotides recited above; (b) a polynucleotide encoding any one of the full length protein, the mature protein, or the soluble form of the protein of GenBank Accession No. NM_{—}000899; (c) a polynucleotide that is an allelic variant of any of the polynucleotides recited above; (d) a polynucleotide that encodes a species homolog of any of the proteins recited above; or (e) a polynucleotide that encodes a polypeptide comprising a specific domain or truncation of the polypeptide of GenBank Accession No. NM_{—}000899. Domains of interest may depend on the nature of the encoded polypeptide; e.g., domains in receptor-like polypeptides include ligand-binding, extracellular, transmembrane, or cytoplasmic domains, or combinations thereof; domains in immunoglobulin-like proteins include the variable immunoglobulin-like domains; domains in enzyme-like polypeptides include catalytic and substrate binding domains; and domains in ligand polypeptides include receptor-binding domains.

The invention is based on the discovery of a c-Kit or (c-Kit)-like polypeptide, the polynucleotides encoding the c-Kit or (c-Kit)-like polypeptide and the use of these compositions for modulating angiogenesis. The c-Kit proto-oncogene encodes the type III receptor tyrosine kinase KIT, which consists of an extracellular ligand binding domain, a transmembrane domain, a negative regulatory juxtamembrane domain and a split kinase domain (Yarden Y. et al. 1987 EMBO 6:3341-3351).

The isolated polynucleotides of the invention include, but are not limited to a polynucleotide comprising any of the nucleotide sequences of GenBank Accession No. X06182; a fragment of GenBank Accession No. X06182; a polynucleotide of GenBank Accession No. X06182 encoding the full length protein; a polynucleotide of GenBank Accession No. X06182 encoding the mature protein; and a polynucleotide of GenBank Accession No. X06182 encoding a soluble form of the protein, e.g., the extracellular ligand-binding domain. The polynucleotides of the present invention also include, but are not limited to, a polynucleotide that hybridizes under stringent conditions to (a) the complement of any of the polynucleotides recited above; (b) a polynucleotide encoding any one of the full length protein, the mature protein, or a soluble form of the protein of GenBank Accession No. X06182; (c) a polynucleotide that is an allelic variant of any of the polynucleotides recited above; (d) a polynucleotide that encodes a species homolog of any of the proteins recited above; or (e) a polynucleotide that encodes a polypeptide comprising a specific domain or truncation of the polypeptide of GenBank Accession No. X06182. Domains of interest may depend on the nature of the encoded polypeptide; e.g., domains in receptor-like polypeptides include ligand-binding, extracellular, transmembrane, or cytoplasmic domains, or combinations thereof; domains in immunoglobulin-like proteins include the variable immunoglobulin-like domains; domains in enzyme-like polypeptides include catalytic and substrate binding domains; and domains in ligand polypeptides include receptor-binding domains.

The polynucleotides of the invention include naturally occurring or wholly or partially synthetic DNA, e.g., cDNA and genomic DNA, and RNA, e.g., mRNA. The polynucleotides may include all of the coding region of the cDNA or may represent a portion of the coding region of the cDNA.

The present invention also provides genes corresponding to the cDNA sequences disclosed herein. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. Further 5′ and 3′ sequence can be obtained using methods known in the art. For example, full length cDNA or genomic DNA that corresponds to any of the polynucleotides recited above can be obtained by screening appropriate cDNA or genomic DNA libraries under suitable hybridization conditions using the polynucleotides recited above or a portion thereof as a probe. Alternatively, any of the polynucleotides recited above may be used as the basis for suitable primer(s) that allow identification and/or amplification of genes in appropriate genomic DNA or cDNA libraries.

The nucleic acid sequences of the invention can be assembled from ESTs and sequences (including cDNA and genomic sequences) obtained from one or more public databases, such as dbEST, gbpri, and UniGene. The EST sequences can provide identifying sequence information, representative fragment or segment information, or novel segment information for the full-length gene.

The polynucleotides of the invention also provide polynucleotides including nucleotide sequences that are substantially equivalent to the polynucleotides recited above. Polynucleotides according to the invention can have, e.g., at least about 65%, at least about 70%, at least about 75%, at least about 80%, 81%, 82%, 83%, 84%, more typically at least about 85%, 86%, 87%, 88%, 89%, more typically at least about 90%, 91%, 92%, 93%, 94%, and even more typically at least about 95%, 96%, 97%, 98%, 99% sequence identity to a polynucleotide recited above.

Included within the scope of the nucleic acid sequences of the invention are nucleic acid sequence fragments that hybridize under stringent conditions to any of the polynucleotides recited above, or complements thereof, which fragment is greater than about 5 nucleotides, preferably 7 nucleotides, more preferably greater than 9 nucleotides and most preferably greater than 17 nucleotides. Fragments of, e.g., 15, 17, or 20 nucleotides or more that are selective for (i.e., specifically hybridize to any one of the polynucleotides of the invention) are contemplated. Probes capable of specifically hybridizing to a polynucleotide can differentiate polynucleotide sequences of the invention from other polynucleotide sequences in the same family of genes or can differentiate human genes from genes of other species, and are preferably based on unique nucleotide sequences.

The sequences falling within the scope of the present invention are not limited to these specific sequences, but also include allelic and species variations thereof. Allelic and species variations can be routinely determined by comparing the sequence provided in any of the polynucleotides recited above, a representative fragment thereof, or a nucleotide sequence at least 90% identical, preferably 95% identical to any of the polynucleotides recited above with a sequence from another isolate of the same species. Furthermore, to accommodate codon variability, the invention includes nucleic acid molecules coding for the same amino acid sequences as do the specific ORFs disclosed herein. In other words, in the coding region of an ORF, substitution of one codon for another codon that encodes the same amino acid is expressly contemplated.

The nearest neighbor result for the nucleic acids of the present invention, including any of the polynucleotides recited above, can be obtained by searching a database using an algorithm or a program. Preferably, a BLAST, which stands for Basic Local Alignment Search Tool, is used to search for local sequence alignments (Altschul, S. P. 1993 J. Mol. Evol. 36:290-300 and Altschul S. F. et al. 1990 J. Mol. Biol. 215:403-410)

Species homologs (or orthologs) of the disclosed polynucleotides and proteins are also provided by the present invention. Species homologs may be isolated and identified by making suitable probes or primers from the sequences provided herein and screening a suitable nucleic acid source from the desired species.

The invention also encompasses allelic variants of the disclosed polynucleotides or proteins; that is, naturally-occurring alternative forms of the isolated polynucleotides that also encode proteins that are identical, homologous or related to that encoded by the polynucleotides.

The nucleic acid sequences of the invention are further directed to sequences that encode variants of the described nucleic acids. These amino acid sequence variants may be prepared by methods known in the art by introducing appropriate nucleotide changes into a native or variant polynucleotide. There are two variables in the construction of amino acid sequence variants: the location of the mutation and the nature of the mutation. Nucleic acids encoding the amino acid sequence variants are preferably constructed by mutating the polynucleotide to encode an amino acid sequence that does not occur in nature. These nucleic acid alterations can be made at sites that differ in the nucleic acids from different species (variable positions) or in highly conserved regions (constant regions). Sites at such locations will typically be modified in series, e.g., by substituting first with conservative choices (e.g., hydrophobic amino acid to a different hydrophobic amino acid) and then with more distant choices (e.g., hydrophobic amino acid to a charged amino acid), and then deletions or insertions may be made at the target site. Amino acid sequence deletions generally range from about 1 to 30 residues, preferably about 1 to 10 residues, and are typically contiguous. Amino acid insertions include amino- and/or carboxyl-terminal fusions ranging in length from one to one hundred or more residues, as well as intrasequence insertions of single or multiple amino acid residues. Intrasequence insertions may range generally from about 1 to 10 amino residues, preferably from 1 to 5 residues. Examples of terminal insertions include the heterologous signal sequences necessary for secretion or for intracellular targeting in different host cells and sequences such as FLAG or poly-histidine sequences useful for purifying the expressed protein.

In a preferred method, polynucleotides encoding the novel amino acid sequences are changed via site-directed mutagenesis. This method uses oligonucleotide sequences to alter a polynucleotide to encode the desired amino acid variant, as well as sufficient adjacent nucleotides on both sides of the changed amino acid to form a stable duplex on either side of the site being changed. In general, the techniques of site-directed mutagenesis are well known to those of skill in the art and this technique is exemplified by publications such as, Adelman et al. 1983 DNA 2:183-93. A versatile and efficient method for producing site-specific changes in a polynucleotide sequence was published by Zoller and Smith 1982 Nucleic Acids Res. 10:6487-6500. PCR may also be used to create amino acid sequence variants of the subject nucleic acids. When small amounts of template DNA are used as starting material, primer(s) that differs slightly in sequence from the corresponding region in the template DNA can generate the desired amino acid variant. PCR amplification results in a population of product DNA fragments that differ from the polynucleotide template encoding the polypeptide at the position specified by the primer. The product DNA fragments replace the corresponding region in the plasmid and this gives a polynucleotide encoding the desired amino acid variant.

A further technique for generating amino acid variants is the cassette mutagenesis technique described in Wells et al. 1985 Gene 34:315-23; and other mutagenesis techniques well known in the art, such as, for example, the techniques in Sambrook J et al. 1989 Molecular Cloning: A Laboratory Manual, Cold Spring Harbor Laboratory, NY, and Current Protocols in Molecular Biology, Ausubel, et al. (eds.) 1992 Current Protocols in Molecular Biology, John Wiley & Sons. Due to the inherent degeneracy of the genetic code, other DNA sequences that encode substantially the same or a functionally equivalent amino acid sequence may be used in the practice of the invention for the cloning and expression of these novel nucleic acids. Such DNA sequences include those that are capable of hybridizing to the appropriate novel nucleic acid sequence under stringent conditions.

Polynucleotides encoding preferred polypeptide truncations of the invention can be used to generate polynucleotides encoding chimeric or fusion proteins comprising one or more domains of the invention and heterologous protein sequences.

The polynucleotides of the invention additionally include the complement of any of the polynucleotides recited above. The polynucleotide can be DNA (genomic, cDNA, amplified, or synthetic) or RNA. Methods and algorithms for obtaining such polynucleotides are well known to those of skill in the art and can include, for example, methods for determining hybridization conditions that can routinely isolate polynucleotides of the desired sequence identities.

In accordance with the invention, any of the polynucleotides recited above, or functional equivalents thereof, may be used to generate recombinant DNA molecules that direct the expression of that nucleic acid, or a functional equivalent thereof, in appropriate host cells. Also included are the cDNA inserts of any of the clones identified herein.

A polynucleotide according to the invention can be joined to any of a variety of other nucleotide sequences by well-established recombinant DNA techniques (see Sambrook J et al. 1989 Molecular Cloning: A Laboratory Manual, Cold Spring Harbor Laboratory, NY). Useful nucleotide sequences for joining to polynucleotides include an assortment of vectors, e.g., plasmids, cosmids, lambda phage derivatives, phagemids, and the like, that are well known in the art. Accordingly, the invention also provides a vector including a polynucleotide of the invention and a host cell containing the polynucleotide. In general, the vector contains an origin of replication functional in at least one organism, convenient restriction endonuclease sites, and a selectable marker for the host cell. Vectors according to the invention include expression vectors, replication vectors, probe generation vectors, and sequencing vectors. A host cell according to the invention can be a prokaryotic or eukaryotic cell and can be a unicellular organism or part of a multicellular organism.

The present invention further provides recombinant constructs comprising a nucleic acid having any of the polynucleotides recited above or a fragment thereof or any other polynucleotides of the invention. In one embodiment, the recombinant constructs of the present invention comprise a vector, such as a plasmid or viral vector, into which any of the polynucleotides recited above or a fragment thereof is inserted in a forward or reverse orientation. In the case of a vector comprising one of the ORFs of the present invention, the vector may further comprise regulatory sequences, including for example, a promoter, operably linked to the ORF. Large numbers of suitable vectors and promoters are known to those of skill in the art and are commercially available for generating the recombinant constructs of the present invention. The following vectors are provided by way of example. Bacterial: pBS, phagescript, PsiXI74, pBluescript SK, pBS KS, pNH8a, pNH16a, pNH18a, pNH46a (Stratagene); pTrc99A, pKK223-3, pKK233-3, pDR540, pRIT5 (Pharmacia). Eukaryotic: pWLneo, pSV2cat, pOG44, PXTI, pSG (Stratagene) pSVK3, pBPV, pMSG, pSVL (Pharmacia).

The isolated polynucleotide of the invention may be operably linked to an expression control sequence such as the pMT2 or pED expression vectors disclosed in Kaufman et al. 1991 Nucleic Acids Res. 19:4485-4490, in order to produce the protein recombinantly. Many suitable expression control sequences are known in the art. General methods of expressing recombinant proteins are also known and are exemplified in Kaufman, R. et al. 1990 Methods in Enzymology 185: 537-566. As defined herein “operably linked” means that the isolated polynucleotide of the invention and an expression control sequence are situated within a vector or cell in such a way that the protein is expressed by a host cell that has been transformed (transfected) with the ligated polynucleotide/expression control sequence.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T1, T7, gpt, lambda PR, and trc. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. Generally, recombinant expression vectors will include origins of replication and selectable markers permitting transformation of the host cell, e.g., the ampicillin resistance gene of E. coli and S. cerevisiae TRP1 gene, and a promoter derived from a highly expressed gene to direct transcription of a downstream structural sequence. Such promoters can be derived from operons encoding glycolytic enzymes such as 3-phosphoglycerate kinase (PGK), α-factor, acid phosphatase, or heat shock proteins, among others. The heterologous structural sequence is assembled in appropriate phase with translation initiation and termination sequences, and preferably, a leader sequence capable of directing secretion of translated protein into the periplasmic space or extracellular medium. Optionally, the heterologous sequence can encode a fusion protein including an amino terminal identification peptide imparting desired characteristics, e.g., stabilization or simplified purification of expressed recombinant product. Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but non-limiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEM 1 (Promega Biotech, Madison, Wis., USA). These pBR322 “backbone” sections are combined with an appropriate promoter and the structural sequence to be expressed. Following transformation of a suitable host strain and growth of the host strain to an appropriate cell density, the selected promoter is induced or derepressed by appropriate means (e.g., temperature shift or chemical induction) and cells are cultured for an additional period. Cells are typically harvested by centrifugation, disrupted by physical or chemical means, and the resulting crude extract retained for further purification.

Host Cells

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell that drives expression of the polynucleotides in the cell.

Knowledge of SCF or SCF-like DNA sequences allows for modification of cells to permit, or increase, expression of SCF or SCF-like polypeptide. Cells can be modified (e.g., by homologous recombination) to provide increased SCF or SCF-like polypeptide expression by replacing, in whole or in part, the naturally occurring SCF or SCF-like promoter with all or part of a heterologous promoter so that the cells SCF or SCF-like polypeptide is expressed at higher levels. The heterologous promoter is inserted in such a manner that it is operatively linked to SCF or SCF-like encoding sequences. See, for example, PCT International Publication No. W094/12650, PCT International Publication No. W092/20808, and PCT International Publication No. W091/09955. It is also contemplated that, in addition to heterologous promoter DNA, amplifiable marker DNA (e.g., ada, dhfr, and the multifunctional CAD gene that encodes carbamyl phosphate synthase, aspartate transcarbamylase, and dihydrorotase) and/or intron DNA may be inserted along with the heterologous promoter DNA. If linked to the SCF or SCF-like coding sequence, amplification of the marker DNA by standard selection methods results in co-amplification of the SCF or SCF-like coding sequences in the cells.

Knowledge of c-Kit or (c-Kit)-like DNA sequences allows for modification of cells to permit, or increase, expression of c-Kit or (c-Kit)-like polypeptide. Cells can be modified (e.g., by homologous recombination) to provide increased c-Kit or (c-Kit)-like polypeptide expression by replacing, in whole or in part, the naturally occurring c-Kit or (c-Kit)-like promoter with all or part of a heterologous promoter so that the cells c-Kit or (c-Kit)-like polypeptide is expressed at higher levels. The heterologous promoter is inserted in such a manner that it is operatively linked to c-Kit or (c-Kit)-like encoding sequences. See, for example, PCT International Publication No. W094/12650, PCT International Publication No. W092/20808, and PCT International Publication No. W091/09955. It is also contemplated that, in addition to heterologous promoter DNA, amplifiable marker DNA (e.g., ada, dhfr, and the multifunctional CAD gene that encodes carbamyl phosphate synthase, aspartate transcarbamylase, and dihydrorotase) and/or intron DNA may be inserted along with the heterologous promoter DNA. If linked to the c-Kit or (c-Kit)-like coding sequence, amplification of the marker DNA by standard selection methods results in co-amplification of the c-Kit or (c-Kit)-like coding sequences in the cells.

The host cell can be a higher eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected by calcium phosphate transfection, DEAE, dextran mediated transfection, or electroporation (Davis, L. et al. 1986 Basic Methods in Molecular Biology, McGraw-Hill). The host cells containing one of polynucleotides of the invention can be used in conventional manners to produce the gene product encoded by the isolated fragment (in the case of an ORF) or can be used to produce a heterologous protein under the control of the EMF.

Any host/vector system can be used to express one or more of the ORFs of the present invention. These include, but are not limited to, eukaryotic hosts such as HeLa cells, CV-1 cells, COS cells, and Sf9 cells, as well as prokaryotic host such as E. coli and B. subtilis. The most preferred cells are those that do not normally express the particular polypeptide or protein or that express the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook J et al. 1989 Molecular Cloning: A Laboratory Manual, Cold Spring Harbor Laboratory, NY.

Various mammalian cell culture systems can also be employed to express recombinant protein. Examples of mammalian expression systems include the COS-7 lines of monkey kidney fibroblasts, described by Gluzman, Y. 1981 Cell 23:175-82, and other cell lines capable of expressing a compatible vector, for example, the C127, 3T3, CHO, HeLa and BHK cell lines. Mammalian expression vectors will comprise an origin of replication, a suitable promoter, and also any necessary ribosome binding sites, polyadenylation sites, splice donor and acceptor sites, transcriptional termination sequences, and 5′ flanking nontranscribed sequences. DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. Protein refolding steps can be used, as necessary, in completing configuration of the target protein. Finally, high performance liquid chromatography (HPLC) can be employed for final purification steps. Microbial cells employed in expression of proteins can be disrupted by any convenient method, including freeze-thaw cycling, sonication, mechanical disruption, or use of cell lysing agents.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods. Such regulatory sequences may be comprised of promoters, enhancers, scaffold-attachment regions, negative regulatory elements, transcriptional initiation sites, and regulatory protein binding sites or combinations of said sequences. Alternatively, sequences that affect the structure or stability of the RNA or protein produced may be replaced, removed, added, or otherwise modified by targeting, including polyadenylation signals, mRNA stability elements, splice sites, leader sequences for enhancing or modifying transport or secretion properties of the protein, or other sequences that alter or improve the function or stability of protein or RNA molecules.

The targeting event may be a simple insertion of the regulatory sequence, placing the gene under the control of the new regulatory sequence, e.g., inserting a new promoter or enhancer or both upstream of a gene. Alternatively, the targeting event may be a simple deletion of a regulatory element, such as the deletion of a tissue-specific negative regulatory element. Alternatively, the targeting event may replace an existing element; for example, a tissue-specific enhancer can be replaced by an enhancer that has broader or different cell-type specificity than the naturally occurring elements. Here, the naturally occurring sequences are deleted and new sequences are added. In all cases, the identification of the targeting event may be facilitated by the use of one or more selectable marker genes that are contiguous with the targeting DNA, allowing for the selection of cells in which the exogenous DNA has integrated into the host cell genome. The identification of the targeting event may also be facilitated by the use of one or more marker genes exhibiting the property of negative selection, such that the negatively selectable marker is linked to the exogenous DNA, but configured such that the negatively selectable marker flanks the targeting sequence, and such that a correct homologous recombination event with sequences in the host cell genome does not result in the stable integration of the negatively selectable marker. Markers useful for this purpose include the Herpes Simplex Virus thymidine kinase (TK) gene or the bacterial xanthine-guanine phosphoribosyl-transferase (gpt) gene.

The gene targeting or gene activation techniques that can be used in accordance with this aspect of the invention are more particularly described in U.S. Pat. No. 5,272,071 to Chappel; U.S. Pat. No. 5,578,461 to Sherwin et al.; International Application No. PCT/US92/09627 (W093/09222) by Selden et al.; and International Application No. PCT/US90/06436 (W091/06667) by Skoultchi et al.

Chimeric and Fusion Proteins

The invention also provides SCF or SCF-like chimeric or fusion proteins. As used herein, an SCF or SCF-like “chimeric protein” or “fusion protein” comprises an SCF or SCF-like polypeptide operatively linked to either a different SCF or SCF-like polypeptide or a non-SCF or SCF-like polypeptide. An “SCF or SCF-like polypeptide” refers to a polypeptide having an amino acid sequence corresponding to the SCF or an SCF-like protein, whereas a “non-SCF or SCF-like polypeptide” refers to a polypeptide having an amino acid sequence corresponding to a protein that is not substantially homologous to the SCF or SCF-like protein, e.g., a protein that is different from the SCF-like protein and that is derived from the same or a different organism. Within an SCF or SCF-like fusion protein an SCF or SCF-like polypeptide can correspond to all or a portion of an SCF or SCF-like protein. In one embodiment, an SCF or SCF-like fusion protein comprises at least one biologically active portion of an SCF or SCF-like protein. In another embodiment, an SCF or SCF-like fusion protein comprises at least two biologically active portions of an SCF or SCF-like protein. In yet another embodiment, an SCF or SCF-like fusion protein comprises at least three biologically active portions of an SCF or SCF-like protein. Within the fusion protein, the term “operatively-linked” is intended to indicate that the SCF or SCF-like polypeptide(s) and/or the non-SCF or SCF-like polypeptide are fused in-frame with one another. The non-SCF or SCF-like polypeptide can be fused to the N-terminus or C-terminus of the SCF or SCF-like polypeptide.

In one embodiment, the fusion protein is a GST-SCF or GST-SCF-like fusion protein in which the SCF or SCF-like sequences are fused to the C-terminus of the GST (glutathione S-transferase) sequences. Such fusion proteins can facilitate the purification of recombinant SCF or SCF-like polypeptides.

In another embodiment, the fusion protein is an SCF or SCF-like protein containing a heterologous signal sequence at its N-terminus. In certain host cells (e.g., mammalian host cells), expression and/or secretion of SCF or SCF-like protein can be increased through use of a heterologous signal sequence.

In yet another embodiment, the fusion protein is an SCF or SCF-like-immunoglobulin fusion protein in which the SCF or SCF-like sequences are fused to sequences derived from a member of the immunoglobulin protein family. The SCF or SCF-like-immunoglobulin fusion proteins of the invention can be incorporated into pharmaceutical compositions and administered to a subject to inhibit an interaction between an SCF or SCF-like ligand and its receptor protein on the surface of a cell, thereby to suppress SCF or SCF-like-mediated signal transduction in vivo. The SCF or SCF-like-immunoglobulin fusion proteins can be used to affect the bioavailability of an SCF or SCF-like cognate ligand. Inhibition of the SCF or SCF-like ligand/receptor interaction can be useful for modulating angiogenesis and as a control for both the treatment of proliferative and differentiative disorders, as well as modulating (e.g., promoting or inhibiting) cell survival. Moreover, the SCF or SCF-like-immunoglobulin fusion proteins of the invention can be used as immunogens to produce anti-SCF or SCF-like antibodies in a subject, to purify SCF or SCF-like ligands, and in screening assays to identify molecules that inhibit the interaction of SCF or SCF-like ligand with its cognate receptor.

An SCF or SCF-like chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) 1992 Current Protocols in Molecular Biology, John Wiley & Sons). Moreover, many expression vectors are commercially available that already encode a fusion moiety (e.g., a GST polypeptide). An SCF or SCF-like polypeptide-encoding nucleic acid can be cloned into such an expression vector such that the fusion moiety is linked in-frame to the SCF or SCF-like protein.

The invention also provides c-Kit or (c-Kit)-like chimeric or fusion proteins. As used herein, a c-Kit or (c-Kit)-like “chimeric protein” or “fusion protein” comprises a c-Kit or (c-Kit)-like polypeptide operatively linked to either a different c-Kit or (c-Kit)-like polypeptide or a non-(c-Kit) or (c-Kit)-like polypeptide. A “c-Kit or (c-Kit)-like polypeptide” refers to a polypeptide having an amino acid sequence corresponding to the c-Kit or a (c-Kit)-like protein, whereas a “non-(c-Kit) or (c-Kit)-like polypeptide” refers to a polypeptide having an amino acid sequence corresponding to a protein that is not substantially homologous to the c-Kit or (c-Kit)-like protein, e.g., a protein that is different from the c-Kit-like protein and that is derived from the same or a different organism. Within a c-Kit or (c-Kit)-like fusion protein a c-Kit or (c-Kit)-like polypeptide can correspond to all or a portion of a c-Kit or (c-Kit)-like protein. In one embodiment, a c-Kit or (c-Kit)-like fusion protein comprises at least one biologically active portion of a c-Kit or (c-Kit)-like protein. In another embodiment, a c-Kit or (c-Kit)-like fusion protein comprises at least two biologically active portions of a c-Kit or (c-Kit)-like protein. In yet another embodiment, a c-Kit or (c-Kit)-like fusion protein comprises at least three biologically active portions of a c-Kit or (c-Kit)-like protein. Within the fusion protein, the term “operatively-linked” is intended to indicate that the c-Kit or (c-Kit)-like polypeptide(s) and/or the non-(c-Kit) or (c-Kit)-like polypeptide are fused in-frame with one another. The non-(c-Kit) or (c-Kit)-like polypeptide can be fused to the N-terminus or C-terminus of the c-Kit or (c-Kit)-like polypeptide.

In one embodiment, the fusion protein is a GST-c-Kit) or GST-(c-Kit)-like fusion protein in which the c-Kit or (c-Kit)-like sequences are fused to the C-terminus of the GST (glutathione S-transferase) sequences. Such fusion proteins can facilitate the purification of recombinant c-Kit or (c-Kit)-like polypeptides.

In another embodiment, the fusion protein is a c-Kit or (c-Kit)-like protein containing a heterologous signal sequence at its N-terminus. In certain host cells (e.g., mammalian host cells), expression and/or secretion of c-Kit or (c-Kit)-like protein can be increased through use of a heterologous signal sequence.

In yet another embodiment, the fusion protein is a c-Kit or (c-Kit)-like-immunoglobulin fusion protein in which the c-Kit or (c-Kit)-like sequences are fused to sequences derived from a member of the immunoglobulin protein family. The c-Kit or (c-Kit)-like-immunoglobulin fusion proteins of the invention can be incorporated into pharmaceutical compositions and administered to a subject to inhibit an interaction between a c-Kit or (c-Kit)-like ligand and its receptor protein on the surface of a cell, thereby to suppress c-Kit or (c-Kit)-like-mediated signal transduction in vivo. The c-Kit or (c-Kit)-like-immunoglobulin fusion proteins can be used to affect the bioavailability of a c-Kit or (c-Kit)-like cognate ligand. Inhibition of the c-Kit or (c-Kit)-like ligand/receptor interaction can be useful for modulating angiogenesis. Moreover, the c-Kit or (c-Kit)-like-immunoglobulin fusion proteins of the invention can be used as immunogens to produce anti-c-Kit or (c-Kit)-like antibodies in a subject, to purify c-Kit or (c-Kit)-like ligands, and in screening assays to identify molecules that inhibit the interaction of c-Kit or (c-Kit)-like ligand with its cognate receptor.

A c-Kit or (c-Kit)-like chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) 1992 Current Protocols in Molecular Biology, John Wiley & Sons). Moreover, many expression vectors are commercially available that already encode a fusion moiety (e.g., a GST polypeptide). A c-Kit or (c-Kit)-like polypeptide-encoding nucleic acid can be cloned into such an expression vector such that the fusion moiety is linked in-frame to the c-Kit or (c-Kit)-like protein.

### POLYPEPTIDES OF THE INVENTION

The isolated polypeptides of the invention include, but are not limited to, a polypeptide comprising: the amino acid sequence set forth as any one of the full length protein, the mature protein, or the soluble form of the protein of GenBank Accession No. NM_{—}000899; or an amino acid sequence encoded by a polynucleotide comprising any of the nucleotide sequences of GenBank Accession No. NM_{—}000899; a fragment of GenBank Accession No. NM_{—}000899; a polynucleotide of GenBank Accession No. NM_{—}000899 encoding the full length protein; a polynucleotide of GenBank Accession No. NM_{—}000899 encoding the mature protein; and a polynucleotide of GenBank Accession No. NM_{—}000899 encoding the soluble form of the protein. Polypeptides of the invention also include polypeptides preferably with biological or immunological activity that are encoded by: (a) a polynucleotide comprising any of the nucleotide sequences of GenBank Accession No. NM_{—}000899; a fragment of GenBank Accession No. NM_{—}000899; a polynucleotide of GenBank Accession No. NM_{—}000899 encoding the full length protein; a polynucleotide of GenBank Accession No. NM_{—}000899 encoding the mature protein; and a polynucleotide of GenBank Accession No. NM_{—}000899 encoding the soluble form of the protein; (b) a polynucleotide encoding any one of the full length protein, the mature protein, or the soluble form of the protein of GenBank Accession No. NM_{—}000899; or (c) polynucleotides that hybridize to the complement of the polynucleotides of either (a) or (b) under stringent hybridization conditions. The invention also provides biologically active or immunologically active variants of any of the amino acid sequences set forth as the full length protein, the mature protein, or the soluble form of the protein of GenBank Accession No. NM_{—}000899; and “substantial equivalents” thereof (e.g., with at least about 65%, at least about 70%, at least about 75%, at least about 80%, at least about 85%, 86%, 87 88%, 89%, at least about 90%, 91%, 92%, 93%, 94%, typically at least about 95%, 96%, 97%, more typically at least about 98%, or most typically at least about 99% amino acid, identity) that retain biological activity. Polypeptides encoded by allelic variants may have a similar, increased, or decreased activity compared to polypeptides set forth as any one of the full length protein, the mature protein, or the soluble form of the protein of GenBank Accession No. NM_{—}000899.

The isolated polypeptides of the invention also include, but are not limited to, a polypeptide comprising: the amino acid sequence set forth as any one of the full length protein, the mature protein, or a soluble form of the protein of GenBank Accession No. X06182; or an amino acid sequence encoded by a polynucleotide comprising any of the nucleotide sequences of GenBank Accession No. X06182; a fragment of GenBank Accession No. X06182; a polynucleotide of GenBank Accession No. X06182 encoding the full length protein; a polynucleotide of GenBank Accession No. X06182 encoding the mature protein; and a polynucleotide of GenBank Accession No. X06182 encoding a soluble form of the protein. Polypeptides of the invention also include polypeptides preferably with biological or immunological activity that are encoded by: (a) a polynucleotide comprising any of the nucleotide sequences of GenBank Accession No. X06182; a fragment of GenBank Accession No. X06182; a polynucleotide of GenBank Accession No. X06182 encoding the full length protein; a polynucleotide of GenBank Accession No. X06182 encoding the mature protein; and a polynucleotide of GenBank Accession No. X06182 encoding a soluble form of the protein; (b) a polynucleotide encoding any one of the full length protein, the mature protein, or a soluble form of the protein of GenBank Accession No. X06182; or (c) polynucleotides that hybridize to the complement of the polynucleotides of either (a) or (b) under stringent hybridization conditions. The invention also provides biologically active or immunologically active variants of any of the amino acid sequences set forth as the full length protein, the mature protein, or a soluble form of the protein of GenBank Accession No. X06182; and “substantial equivalents” thereof (e.g., with at least about 65%, at least about 70%, at least about 75%, at least about 80%, at least about 85%, 86%, 87 88%, 89%, at least about 90%, 91%, 92%, 93%, 94%, typically at least about 95%, 96%, 97%, more typically at least about 98%, or most typically at least about 99% amino acid, identity) that retain biological activity. Polypeptides encoded by allelic variants may have a similar, increased, or decreased activity compared to polypeptides set forth as any one of the full length protein, the mature protein, or a soluble form of the protein of GenBank Accession No. X06182.

Fragments of the proteins of the present invention that are capable of exhibiting biological activity are also encompassed by the present invention. Fragments of the protein may be in linear form or they may be cyclized using known methods, for example, as described in H. U. Saragovi, et al. 1992 Bio/Technology 10: 773-778, and R. S. McDowell et al. 1992 J. Amer. Chem. Soc. 114: 9245-9253. Such fragments may be fused to carrier molecules such as immunoglobulins for many purposes, including increasing the valency of protein binding sites.

The present invention also provides full length transmembrane, mature, and soluble forms of the disclosed protein. The protein coding sequence is identified in the GenBank listing by translation of the disclosed nucleotide sequence. The mature form of such protein may be obtained by expression of a full-length polynucleotide in a suitable mammalian cell or other host cell. The sequence of the mature form of the protein is also determinable from the amino acid sequence of the full length form, for example, by omission of the signal peptide. Since proteins of the present invention are soluble, membrane bound forms of the proteins are also provided.

Protein compositions of the present invention may further comprise an acceptable carrier, such as a hydrophilic, e.g., pharmaceutically acceptable, carrier.

The present invention further provides isolated polypeptides encoded by the nucleic acid fragments of the present invention or by degenerate variants of the nucleic acid fragments of the present invention. By “degenerate variant” is intended nucleotide fragments that differ from a nucleic acid fragment of the present invention (e.g., an ORF) by nucleotide sequence but, due to the degeneracy of the genetic code, encode an identical polypeptide sequence. Preferred nucleic acid fragments of the present invention are the ORFs that encode proteins.

A variety of methodologies known in the art can be utilized to obtain any one of the isolated polypeptides or proteins of the present invention. At the simplest level, the amino acid sequence can be synthesized using commercially available peptide synthesizers. The synthetically-constructed protein sequences, by virtue of sharing primary, secondary or tertiary structural and/or conformational characteristics with proteins may possess biological properties in common therewith, including protein activity. This technique is particularly useful in producing small peptides and fragments of larger polypeptides. Fragments are useful, for example, in generating antibodies against the native polypeptide. Thus, they may be employed as biologically active or immunological substitutes for natural, purified proteins in screening of therapeutic compounds and in immunological processes for the development of antibodies.

The polypeptides and proteins of the present invention can alternatively be purified from cells that have been altered to express the desired polypeptide or protein. As used herein, a cell is said to be altered to express a desired polypeptide or protein when the cell, through genetic manipulation, is made to produce a polypeptide or protein that it normally does not produce or that the cell normally produces at a lower level. One skilled in the art can readily adapt procedures for introducing and expressing either recombinant or synthetic sequences into eukaryotic or prokaryotic cells in order to generate a cell that produces one of the polypeptides or proteins of the present invention.

The invention also relates to methods for producing a polypeptide comprising growing a culture of host cells of the invention in a suitable culture medium, and purifying the protein from the cells or the culture in which the cells are grown. For example, the methods of the invention include a process for producing a polypeptide in which a host cell containing a suitable expression vector that includes a polynucleotide of the invention is cultured under conditions that allow expression of the encoded polypeptide. The polypeptide can be recovered from the culture, conveniently from the culture medium, or from a lysate prepared from the host cells and further purified. Preferred embodiments include those in which the protein produced by such process is a full length, mature, or soluble form of the protein.

In an alternative method, the polypeptide or protein is purified from bacterial cells that naturally produce the polypeptide or protein. One skilled in the art can readily follow known methods for isolating polypeptides and proteins in order to obtain one of the isolated polypeptides or proteins of the present invention. These include, but are not limited to, immunochromatography, HPLC, size-exclusion chromatography, ion-exchange chromatography, and immuno-affinity chromatography. See, e.g., Scopes, Protein Purification: Principles and Practice, Springer-Verlag (1994); Sambrook J et al. 1989 Molecular Cloning: A Laboratory Manual, Cold Spring Harbor Laboratory, NY; Ausubel, et al. (eds. ) 1992 Current Protocols in Molecular Biology, John Wiley & Sons. Polypeptide fragments that retain biological activity include fragments comprising greater than about 100 amino acids, or greater than about 200 amino acids, and fragments that encode specific protein domains.

The proteins of the invention may also be expressed as a product of transgenic animals, e.g., as a component of the milk of transgenic cows, goats, pigs, or sheep that are characterized by somatic or germ cells containing a nucleotide sequence encoding the protein.

The proteins provided herein also include proteins characterized by amino acid sequences similar to those of purified proteins but into which modification are naturally provided or deliberately engineered. For example, modifications, in the peptide or DNA sequence, can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein. Regions of the protein that are important for the protein function can be determined by various methods known in the art including the alanine-scanning method that involves systematic substitution of single or strings of amino acids with alanine, followed by testing the resulting alanine-containing variant for biological activity. This type of analysis determines the importance of the substituted amino acid(s) in biological activity. Regions of the protein that are important for protein function cay be determined by the eMATRIX program.

The proteins may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBat™ kit), and such methods are well known in the art, as described in Summers and Smith 1987 Texas Agricultural Experiment Station Bulletin No. 1555. As used herein, an insect cell capable of expressing a polynucleotide of the present invention is “transformed”.

The protein of the invention may be prepared by culturing transfected host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents that will bind to the protein; one or more column steps over such affinity resins as concanavalin A-agarose, heparin-toyopearl™ or Cibacrom blue 3GA Sepharose™; one or more steps involving hydrophobic interaction chromatography using such resins as phenyl ether, butyl ether, or propyl ether; or immunoaffinity chromatography.

Alternatively, the protein of the invention may also be expressed in a form that will facilitate purification. For example, it may be expressed as a fusion protein, such as those of maltose binding protein (MBP), glutathione-S-transferase (GST) or thioredoxin (TRX), or as a His tag. Kits for expression and purification of such fusion proteins are commercially available from New England BioLabs (Beverly, Mass.), Pharmacia (Piscataway, N.J.) and Invitrogen, respectively. The protein can also be tagged with an epitope and subsequently purified by using a specific antibody directed to such epitope. One such epitope (“FLAG”®) is commercially available from Kodak (New Haven, Conn.).

Finally, one or more reverse-phase high performance liquid chromatography (RP-HPLC) steps employing hydrophobic, RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein”.

The polypeptides of the invention include analogs (variants). The polypeptides of the invention include SCF-like analogs. This embraces fragments of SCF or SCF-like polypeptides of the invention, as well as SCF or SCF-like polypeptides that comprise one or more amino acids deleted, inserted, or substituted. Also, analogs of the SCF or SCF-like polypeptides of the invention embrace fusions of the SCF or SCF-like polypeptides or modifications of the SCF or SCF-like polypeptides, wherein the SCF or SCF-like polypeptide or analog is fused to another moiety or moieties, e.g., targeting moiety or another therapeutic agent. Such analogs may exhibit improved properties such as activity and/or stability. Examples of moieties that may be fused to the SCF or SCF-like polypeptide or an analog include, for example, targeting moieties that provide for the delivery of polypeptide to neurons, e.g., antibodies to central nervous system, or antibodies to receptor and ligands expressed on neuronal cells. Other moieties that may be fused to SCF or SCF-like polypeptides include therapeutic agents that are used for treatment, for example antidepressant drugs or other medications for neurological disorders. Also, SCF or SCF-like polypeptides may be fused to neuron growth modulators, and other chemokines for targeted delivery.

The polypeptides of the invention include (c-Kit)-like analogs. This embraces fragments of (c-Kit) or (c-Kit)-like polypeptides of the invention, as well as (c-Kit) or (c-Kit)-like polypeptides that comprise one or more amino acids deleted, inserted, or substituted. Also, analogs of the (c-Kit) or (c-Kit)-like polypeptides of the invention embrace fusions of the (c-Kit) or (c-Kit)-like polypeptides or modifications of the (c-Kit) or (c-Kit)-like polypeptides, wherein the (c-Kit) or (c-Kit)-like polypeptide or analog is fused to another moiety or moieties, e.g., targeting moiety or another therapeutic agent. Such analogs may exhibit improved properties such as activity and/or stability.

Antibodies to SCF or c-Kit Proteins

Antibodies that specifically recognize one or more epitopes of SCF or c-Kit, or epitopes of conserved variants of SCF or c-Kit, or peptide fragments of SCF or c-Kit are also encompassed by the invention. Such antibodies include but are not limited to polyclonal antibodies, monoclonal antibodies (mAbs), humanized or chimeric antibodies, single chain antibodies, Fab fragments, F(ab′)₂ fragments, fragments produced by a Fab expression library, anti-idiotypic (anti-Id) antibodies, and epitope-binding fragments of any of the above.

The antibodies of the invention may be used, for example, in the detection of SCF or c-Kit in a biological sample and may, therefore, be utilized as part of a diagnostic or prognostic technique whereby patients may be tested for abnormal amounts of SCF or c-Kit. Such antibodies may also be utilized in conjunction with, for example, compound screening schemes, as described below, for the evaluation of the effect of test compounds on expression and/or activity of the SCF or c-Kit gene product. Additionally, such antibodies can be used in conjunction with the gene therapy techniques described to, for example, evaluate the normal and/or engineered SCF or c-Kit-expressing cells prior to their introduction into the patient. Such antibodies may additionally be used as a method for the inhibition of abnormal SCF or c-Kit activity. Thus, such antibodies may, therefore, be utilized as part of treatment methods to modulate angiogenesis.

For the production of antibodies, various host animals may be immunized by injection with SCF or c-Kit, an SCF or c-Kit peptide (e.g., one corresponding to the functional domain of the receptor, such as ECD, TM or CD), truncated SCF or c-Kit polypeptides (c-Kit in which one or more domains, e.g., the TM or CD, has been deleted), functional equivalents of SCF or c-Kit or mutants of SCF or c-Kit. Such host animals may include but are not limited to rabbits, mice, and rats, to name but a few. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Polyclonal antibodies are heterogeneous populations of antibody molecules derived from the sera of the immunized animals.

Monoclonal antibodies, which are homogeneous populations of antibodies to a particular antigen, may be obtained by any technique that provides for the production of antibody molecules by continuous cell lines in culture. These include, but are not limited to, the hybridoma technique of Kohler and Milstein, (1975, Nature 256:495-497; and U.S. Pat. No. 4,376,110), the human B-cell hybridoma technique (Kosbor et al., 1983 Immunology Today 4:72; Cole et al., 1983 Proc Natl Acad Sci USA 80:2026-2030), and the EBV-hybridoma technique (Cole et al., 1985 in Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mAbs in vivo makes this the presently preferred method of production.

In addition, techniques developed for the production of “chimeric antibodies” (Morrison et al. 1984 Proc Natl Acad Sci USA 81:6851-6855; Neuberger et al. 1984 Nature 312:604-608; Takeda et al. 1985 Nature 314:452-454) by splicing the genes from a mouse antibody molecule of appropriate antigen specificity together with genes from a human antibody molecule of appropriate biological activity can be used. A chimeric antibody is a molecule in which different portions are derived from different animal species, such as those having a variable region derived from a murine mAb and a human immunoglobulin constant region.

Alternatively, techniques described for the production of single chain antibodies (U.S. Pat. No. 4,946,778; Bird 1988 Science 242:423-426; Huston et al. 1988 Proc Natl Acad Sci USA 85:5879-5883; and Ward et al. 1989 Nature 334:544-546) can be adapted to produce single chain antibodies against SCF or c-Kit gene products. Single chain antibodies are formed by linking the heavy and light chain fragments of the Fv region via an amino acid bridge, resulting in a single chain polypeptide.

Antibody fragments which recognize specific epitopes may be generated by known techniques. For example, such fragments include but are not limited to: the F(ab′)₂ fragments that can be produced by pepsin digestion of the antibody molecule and the Fab fragments that can be generated by reducing the disulfide bridges of the F(ab′)₂ fragments. Alternatively, Fab expression libraries may be constructed (Huse et al., 1989 Science 246:1275-1281) to allow rapid and easy identification of monoclonal Fab fragments with the desired specificity.

Antibodies to SCF or c-Kit can, in turn, be utilized to generate anti-idiotype antibodies that “mimic” SCF or c-Kit, using techniques well known to those skilled in the art. (See, e.g., Greenspan & Bona, 1993 FASEB J 7:437-444; and Nissinoff, 1991 J Immunol 147:2429-2438). For example antibodies that bind to the c-Kit ECD and competitively inhibit the binding of SCF to the c-Kit can be used to generate anti-idiotypes that “mimic” the ECD and, therefore, bind and neutralize c-Kit. Such neutralizing anti-idiotypes or Fab fragments of such anti-idiotypes can be used in therapeutic regimens to neutralize c-Kit and inhibit angiogenesis.

Screening Assays for Compounds that Modulate SCF or c-Kit Expression or Activity

The following assays are designed to identify compounds that interact with (e.g., bind to) SCF or c-Kit (including, but not limited to the ECD or CD of c-Kit), compounds that interact with (e.g., bind to) intracellular proteins that interact with c-Kit (including, but not limited to, the TM and CD of c-Kit), compounds that interfere with the interaction of c-Kit with transmembrane or intracellular proteins involved in c-Kit-mediated signal transduction, and to compounds that modulate the activity of SCF or c-Kit genes (i.e., modulate the level of SCF or c-Kit gene expression) or modulate the level of SCF or c-Kit. Assays may additionally be utilized that identify compounds that bind to SCF or c-Kit gene regulatory sequences (e.g., promoter sequences) and that may modulate SCF or c-Kit gene expression.

The compounds which may be screened in accordance with the invention include, but are not limited to peptides, antibodies and fragments thereof, and other organic compounds (e.g., peptidomimetics) that bind to the ECD of c-Kit and either mimic the activity triggered by SCF (i.e., agonists) or inhibit the activity triggered by SCF (i.e., antagonists); as well as peptides, antibodies or fragments thereof, and other organic compounds that mimic the ECD of c-Kit (or a portion thereof) and bind to and “neutralize” SCF.

Such compounds may include, but are not limited to, peptides such as, for example, soluble peptides, including but not limited to members of random peptide libraries (see, e.g., Lam, K. S. et al. 1991 Nature 354:82-84; Houghten, R. et al. 1991 Nature 354:84-86), and combinatorial chemistry-derived molecular libraries made of D- and/or L-configuration amino acids, phosphopeptides (including, but not limited to, members of random or partially degenerate, directed phosphopeptide libraries; see, e.g., Songyang, Z. et al. 1993 Cell 72:767-778), antibodies (including, but not limited to, polyclonal, monoclonal, humanized, anti-idiotypic, chimeric or single chain antibodies, and FAb, F(ab′)₂ and FAb expression library fragments, and epitope-binding fragments thereof), and small organic or inorganic molecules.

Other compounds which can be screened in accordance with the invention include but are not limited to small organic molecules that are able to cross the blood-brain barrier, gain entry into an appropriate cell and affect the expression of the SCF or c-Kit genes or some other gene involved in the SCF/c-Kit signal transduction pathway (e.g., by interacting with the regulatory region or transcription factors involved in gene expression); or such compounds that affect the activity of SCF/c-Kit (e.g., by inhibiting or enhancing the enzymatic activity of the CD) or the activity of some other intracellular factor involved in the SCF/c-Kit signal transduction pathway.

Computer modeling and searching technologies permit identification of compounds, or the improvement of already identified compounds, that can modulate SCF or c-Kit expression or activity. Having identified such a compound or composition, the active sites or regions are identified. Such active sites might typically be ligand binding sites, such as the interaction domains of SCF with c-Kit itself. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found. Next, the three dimensional geometric structure of the active site is determined. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures may be measured with a complexed ligand, natural or artificial, that may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods. Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential SCF or c-Kit modulating compounds.

Alternatively, these methods can be used to identify improved modulating compounds from an already known modulating compound or ligand. The composition of the known compound can be modified and the structural effects of modification can be determined using the experimental and computer modeling methods described above applied to the new composition. The altered structure is then compared to the active site structure of the compound to determine if an improved fit or interaction results. In this manner systematic variations in composition, such as by varying side groups, can be quickly evaluated to obtain modified modulating compounds or ligands of improved specificity or activity.

Further experimental and computer modeling methods useful to identify modulating compounds based upon identification of the active sites of SCF, c-Kit, and related transduction and transcription factors will be apparent to those of skill in the art.

Examples of molecular modeling systems are the CHARMM and QUANTA programs (Polygen Corporation, Waltham, Mass.). CHARMM performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific-proteins, such as Rotivinen, et al. 1988 Acta Pharmaceutical Fennica 97:159-166; Ripka, Jun. 16, 1988 New Scientist 54-57; McKinaly and Rossmann 1989 Annu Rev Pharmacol Toxiciol 29:111-122; Perry and Davies in OSAR: Quantitative Structure-Activity Relationships in Drug Design pp. 189-193 (Alan R. Liss, Inc. 1989); Lewis and Dean 1989 Proc R Soc Lond 236:125-140 and 141-162; and, with respect to a model receptor for nucleic acid components, Askew, et al. 1989 J Am Chem Soc 111:1082-1090. Other computer programs that screen and graphically depict chemicals are available from companies such as BioDesign, Inc. (Saco, Me.), NPS Allelix Corp. (Mississauga, Ontario, Canada), and Hypercube, Inc. (Gainesville, Fla.). Although these are primarily designed for application to drugs specific to particular proteins, they can be adapted to design of drugs specific to regions of DNA or RNA, once that region is identified.

Although described above with reference to design and generation of compounds which could alter binding, one could also screen libraries of known compounds, including natural products or synthetic chemicals, and biologically active materials, including proteins, for compounds that are inhibitors or activators.

Compounds identified via assays such as those described herein may be useful, for example, in elaborating the biological functions of SCF and c-Kit gene products, and for modulating angiogenesis. Assays for testing the effectiveness of compounds are discussed below.

In Vitro Screening Assays for Compounds that Bind to SCF or c-Kit

In vitro systems may be designed to identify compounds capable of interacting with (e.g., binding to) SCF or c-Kit (including, but not limited to, the ECD or CD of c-Kit). Compounds identified may be useful, for example, in modulating the activity of wild type and/or mutant SCF or c-Kit gene products; may be useful in elaborating the biological function of SCF or c-Kit; may be utilized in screens for identifying compounds that disrupt normal SCF/c-Kit interactions; or may in themselves disrupt such interactions.

The principle of the assays used to identify compounds that bind to SCF or c-Kit involves preparing a reaction mixture of the SCF or c-Kit and the test compound under conditions and for a time sufficient to allow the two components to interact and bind, thus forming a complex that can be removed and/or detected in the reaction mixture. The SCF or c-Kit species used can vary depending upon the goal of the screening assay. For example, where agonists of SCF are sought, the full length c-Kit, or a soluble truncated c-Kit, e.g., in which the TM and/or CD is deleted from the molecule, a peptide corresponding to the ECD or a fusion protein containing the c-Kit ECD fused to a protein or polypeptide that affords advantages in the assay system (e.g., labeling, isolation of the resulting complex, etc.) can be utilized. Where compounds that interact with the cytoplasmic domain are sought to be identified, peptides corresponding to the c-Kit CD and fusion proteins containing the c-Kit CD can be used.

The screening assays can be conducted in a variety of ways. For example, one method to conduct such an assay would involve anchoring the SCF or c-Kit protein, polypeptide, peptide or fusion protein or the test substance onto a solid phase and detecting (SCF or c-Kit)/test compound complexes anchored on the solid phase at the end of the reaction. In one embodiment of such a method, the SCF or c-Kit reactant may be anchored onto a solid surface, and the test compound, which is not anchored, may be labeled, either directly or indirectly.

In practice, microtiter plates may conveniently be utilized as the solid phase. The anchored component may be immobilized by non-covalent or covalent attachments. Non-covalent attachment may be accomplished by simply coating the solid surface with a solution of the protein and drying. Alternatively, an immobilized antibody, preferably a monoclonal antibody, specific for the protein to be immobilized may be used to anchor the protein to the solid surface. The surfaces may be prepared in advance and stored.

In order to conduct the assay, the non-immobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously non-immobilized component is pre-labeled, the detection of label immobilized on the surface indicates that complexes were formed. Where the previously non-immobilized component is not pre-labeled, an indirect label can be used to detect complexes anchored on the surface; e.g., using a labeled antibody specific for the previously non-immobilized component (the antibody, in turn, may be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for SCF or c-Kit protein, polypeptide, peptide or fusion protein or the test compound to anchor any complexes formed in solution, and a labeled antibody specific for the other component of the possible complex to detect anchored complexes.

Alternatively, cell-based assays, membrane vesicle-based assays and membrane fraction-based assays can be used to identify compounds that interact with SCF or c-Kit. To this end, cell lines that express SCF or c-Kit, or cell lines (e.g., COS cells, CHO cells, fibroblasts, etc.) that have been genetically engineered to express SCF or c-Kit (e.g., by transfection or transduction of SCF or c-Kit DNA) can be used. Interaction of the test compound with, for example, the ECD of c-Kit expressed by the host cell can be determined by comparison or competition with native SCF.

Assays for Intracellular Proteins that Interact with c-Kit

Any method suitable for detecting protein-protein interactions may be employed for identifying transmembrane proteins or intracellular proteins that interact with c-Kit. Among the traditional methods that may be employed are co-immunoprecipitation, cross-linking and co-purification through gradients or chromatographic columns of cell lysates or proteins obtained from cell lysates and c-Kit to identify proteins in the lysate that interact with c-Kit. For these assays, the c-Kit component used can be a full length c-Kit, a soluble derivative lacking the membrane-anchoring region (e.g., a truncated c-Kit in which the TM is deleted resulting in a truncated molecule containing the ECD fused to the CD), a peptide corresponding to the CD or a fusion protein containing the CD of c-Kit. Once isolated, such an intracellular protein can be identified and can, in turn, be used, in conjunction with standard techniques, to identify proteins with which it interacts. For example, at least a portion of the amino acid sequence of an intracellular protein that interacts with c-Kit can be ascertained using techniques well known to those of skill in the art, such as via the Edman degradation technique. (See, e.g., Creighton, 1983, “Proteins: Structures and Molecular Principles”, W.H. Freeman & Co., N.Y., pp. 34-49). The amino acid sequence obtained may be used as a guide for the generation of oligonucleotide mixtures that can be used to screen for gene sequences encoding such intracellular proteins. Screening may be accomplished, for example, by standard hybridization or PCR techniques. Techniques for the generation of oligonucleotide mixtures and the screening are well-known. (See, e.g., Ausubel, supra., and PCR Protocols: A Guide to Methods and Applications, 1990, Innis, M. et al., eds. Academic Press, Inc., New York).

Additionally, methods may be employed that result in the simultaneous identification of genes that encode the transmembrane or intracellular proteins interacting with c-Kit. These methods include, for example, probing expression libraries, in a manner similar to the well known technique of antibody probing of λgt11 libraries, using labeled c-Kit protein, or a c-Kit polypeptide, peptide or fusion protein, e.g., a c-Kit polypeptide or c-Kit domain fused to a marker (e.g., an enzyme, fluor, luminescent protein, or dye), or an Ig-Fc domain.

One method that detects protein interactions in vivo, the two-hybrid system, is described in detail for illustration only and not by way of limitation. One version of this system has been described (Chien et al., 1991, Proc. Natl. Acad. Sci. USA, 88:9578-9582) and is commercially available from Clontech (Palo Alto, Calif.).

Briefly, utilizing such a system, plasmids are constructed that encode two hybrid proteins: one plasmid consists of nucleotides encoding the DNA-binding domain of a transcription activator protein fused to an SCF or c-Kit nucleotide sequence encoding SCF or c-Kit, an SCF or c-Kit polypeptide, peptide or fusion protein, and the other plasmid consists of nucleotides encoding the transcription activator protein's activation domain fused to a cDNA encoding an unknown protein that has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology may be used to screen activation domain libraries for proteins that interact with the “bait” gene product. By way of example, and not by way of limitation, SCF or c-Kit may be used as the bait gene product. Total genomic or cDNA sequences are fused to the DNA encoding an activation domain. This library and a plasmid encoding a hybrid of a bait SCF or c-Kit gene product fused to the DNA-binding domain are co-transformed into a yeast reporter strain, and the resulting transformants are screened for those that express the reporter gene. For example, and not by way of limitation, a bait SCF or c-Kit gene sequence, such as the open reading frame of SCF or c-Kit (or a domain of SCF or c-Kit) can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait SCF or c-Kit gene product are to be detected can be made using methods routinely practiced in the art. According to the particular system described herein, for example, the cDNA fragments can be inserted into a vector such that they are translationally fused to the transcriptional activation domain of GAL4. This library can be co-transformed along with the bait SCF or c-Kit gene-GAL4 fusion plasmid into a yeast strain which contains a lacZ gene driven by a promoter that contains GAL4 activation sequence. A cDNA encoded protein, fused to GAL4 transcriptional activation domain, that interacts with bait SCF or c-Kit gene product will reconstitute an active GAL4 protein and thereby drive expression of the HIS3 gene. Colonies that express HIS3 can be detected by their growth on petri dishes containing semi-solid agar based media lacking histidine. The cDNA can then be purified from these strains, and used to produce and isolate the bait SCF or c-Kit gene-interacting protein using techniques routinely practiced in the art.

Assays for Compounds that Interfere with c-Kit/Intracellular or c-Kit/Transmembrane Macromolecule Interaction

The macromolecules that interact with the c-Kit are referred to, for purposes of this discussion, as “binding partners”. These binding partners are likely to be involved in the SCF/c-Kit signal transduction pathway, and therefore, in the role of SCF/c-Kit in modulation of angiogenesis. Therefore, it is desirable to identify compounds that interfere with or disrupt the interaction of such binding partners with SCF which may be useful in regulating the activity of c-Kit and modulate angiogenesis associated with SCF/c-Kit activity.

The basic principle of the assay systems used to identify compounds that interfere with the interaction between the c-Kit and its binding partner or partners involves preparing a reaction mixture containing c-Kit protein, polypeptide, peptide or fusion protein and the binding partner under conditions and for a time sufficient to allow the two to interact and bind, thus forming a complex. In order to test a compound for inhibitory activity, the reaction mixture is prepared in the presence and absence of the test compound. The test compound may be initially included in the reaction mixture, or may be added at a time subsequent to the addition of the c-Kit moiety and its binding partner. Control reaction mixtures are incubated without the test compound or with a placebo. The formation of any complexes between the c-Kit moiety and the binding partner is then detected. The formation of a complex in the control reaction, but not in the reaction mixture containing the test compound, indicates that the compound interferes with the interaction of c-Kit and the interactive binding partner. Additionally, complex formation within reaction mixtures containing the test compound and normal c-Kit protein may also be compared to complex formation within reaction mixtures containing the test compound and a mutant c-Kit. This comparison may be important in those cases wherein it is desirable to identify compounds that disrupt interactions of mutant but not normal c-Kit.

The assay for compounds that interfere with the interaction of the c-Kit and binding partners can be conducted in a heterogeneous or homogeneous format. Heterogeneous assays involve anchoring either the c-Kit moiety product or the binding partner onto a solid phase and detecting complexes anchored on the solid phase at the end of the reaction. In homogeneous assays, the entire reaction is carried out in a liquid phase. In either approach, the order of addition of reactants can be varied to obtain different information about the compounds being tested. For example, test compounds that interfere with the interaction by competition can be identified by conducting the reaction in the presence of the test substance; i.e., by adding the test substance to the reaction mixture prior to or simultaneously with the c-Kit moiety and interactive binding partner. Alternatively, test compounds that disrupt preformed complexes, e.g., compounds with higher binding constants that displace one of the components from the complex, can be tested by adding the test compound to the reaction mixture after complexes have been formed. The various formats are described briefly below.

In a heterogeneous assay system, either the c-Kit moiety or the interactive binding partner, is anchored onto a solid surface, while the non-anchored species is labeled, either directly or indirectly. In practice, microtiter plates are conveniently utilized. The anchored species may be immobilized by non-covalent or covalent attachments. Non-covalent attachment may be accomplished simply by coating the solid surface with a solution of the c-Kit gene product or binding partner and drying. Alternatively, an immobilized antibody specific for the species to be anchored may be used to anchor the species to the solid surface. The surfaces may be prepared in advance and stored.

In order to conduct the assay, the partner of the immobilized species is exposed to the coated surface with or without the test compound. After the reaction is complete, unreacted components are removed (e.g., by washing) and any complexes formed will remain immobilized on the solid surface. The detection of complexes anchored on the solid surface can be accomplished in a number of ways. Where the non-immobilized species is pre-labeled, the detection of label immobilized on the surface indicates that complexes were formed. Where the non-immobilized species is not pre-labeled, an indirect label can be used to detect complexes anchored on the surface; e.g., using a labeled antibody specific for the initially non-immobilized species (the antibody, in turn, may be directly labeled or indirectly labeled with a labeled anti-Ig antibody). Depending upon the order of addition of reaction components, test compounds that inhibit complex formation or that disrupt preformed complexes can be detected.

Alternatively, the reaction can be conducted in a liquid phase in the presence or absence of the test compound, the reaction products separated from unreacted components, and complexes detected; e.g., using an immobilized antibody specific for one of the binding components to anchor any complexes formed in solution, and a labeled antibody specific for the other partner to detect anchored complexes. Again, depending upon the order of addition of reactants to the liquid phase, test compounds that inhibit complex or that disrupt preformed complexes can be identified.

In an alternate embodiment of the invention, a homogeneous assay can be used. In this approach, a preformed complex of the c-Kit moiety and the interactive binding partner is prepared in which either the c-Kit or its binding partner is labeled, but the signal generated by the label is quenched due to formation of the complex (see, e.g., U.S. Pat. No. 4,109,496 by Rubenstein that utilizes this approach for immunoassays). The addition of a test substance that competes with and displaces one of the species from the preformed complex will result in the generation of a signal above background. In this way, test substances that disrupt c-Kit/intracellular binding partner interaction can be identified.

In a particular embodiment, a c-Kit fusion can be prepared for immobilization. For example, c-Kit or a peptide fragment, e.g., corresponding to the CD, can be fused to a glutathione-S-transferase (GST) gene using a fusion vector, such as pGEX-5X-1, in such a manner that its binding activity is maintained in the resulting fusion protein. The interactive binding partner can be purified and used to raise a monoclonal antibody, using methods routinely practiced in the art. This antibody can be labeled with the radioactive isotope ¹²⁵I, for example, by methods routinely practiced in the art. In a heterogeneous assay, e.g., the GST-c-Kit fusion protein can be anchored to glutathione-agarose beads. The interactive binding partner can then be added in the presence or absence of the test compound in a manner that allows interaction and binding to occur. At the end of the reaction period, unbound material can be washed away, and the labeled monoclonal antibody can be added to the system and allowed to bind to the complexed components. The interaction between the c-Kit gene product and the interactive binding partner can be detected by measuring the amount of radioactivity that remains associated with the glutathione-agarose beads. A successful inhibition of the interaction by the test compound will result in a decrease in measured radioactivity.

Alternatively, the GST-c-Kit fusion protein and the interactive binding partner can be mixed together in liquid in the absence of the solid glutathione-agarose beads. The test compound can be added either during or after the species are allowed to interact. This mixture can then be added to the glutathione-agarose beads and unbound material is washed away. Again the extent of inhibition of the c-Kit/binding partner interaction can be detected by adding the labeled antibody and measuring the radioactivity associated with the beads.

In another embodiment of the invention, these same techniques can be employed using peptide fragments that correspond to the binding domains of c-Kit and/or the interactive or binding partner (in cases where the binding partner is a protein), in place of one or both of the full length proteins. Any number of methods routinely practiced in the art can be used to identify and isolate the binding sites. These methods include, but are not limited to, mutagenesis of the gene encoding one of the proteins and screening for disruption of binding in a co-immunoprecipitation assay. Compensating mutations in the gene encoding the second species in the complex can then be selected. Sequence analysis of the genes encoding the respective proteins will reveal the mutations that correspond to the region of the protein involved in interactive binding. Alternatively, one protein can be anchored to a solid surface using methods described above, and allowed to interact with and bind to its labeled binding partner, which has been treated with a proteolytic enzyme, such as trypsin. After washing, a short, labeled peptide comprising the binding domain may remain associated with the solid material, which can be isolated and identified by amino acid sequencing. Also, once the gene coding for the intracellular binding partner is obtained, short gene segments can be engineered to express peptide fragments of the protein, which can then be tested for binding activity and purified or synthesized.

For example, and not by way of limitation, a c-Kit gene product can be anchored to a solid material as described, above, by making a GST-c-Kit fusion protein and allowing it to bind to glutathione agarose beads. The interactive binding partner can be labeled with a radioactive isotope, such as ³⁵S, and cleaved with a proteolytic enzyme such as trypsin. Cleavage products can then be added to the anchored GST-c-Kit fusion protein and allowed to bind. After washing away unbound peptides, labeled bound material, representing the intracellular binding partner binding domain, can be eluted, purified, and analyzed for amino acid sequence by well-known methods. Peptides so identified can be produced synthetically or fused to appropriate facilitative proteins using recombinant DNA technology.

Assays for Identification of Compounds that Modulate Angiogenesis

Compounds, including but not limited to binding compounds identified via assay techniques such as those described above, can be tested for the ability to modulate angiogenesis. The assays described above can identify compounds that affect c-Kit activity (e.g., compounds that bind to c-Kit, inhibit or promote binding of SCF, and either activate signal transduction (agonists) or block activation (antagonists), and compounds that bind to SCF and neutralize ligand activity); or compounds that affect SCF or c-Kit gene activity (by affecting SCF or c-Kit gene expression, including molecules, e.g., proteins or small organic molecules, that affect or interfere with splicing events so that expression of the full length or a truncated form of SCF or c-Kit can be modulated). However, it should be noted that the assays described can also identify compounds that modulate SCF/c-Kit signal transduction (e.g., compounds that affect downstream signaling events, such as inhibitors or enhancers of tyrosine kinase activity that participates in transducing the signal activated by SCF binding to c-Kit). The identification and use of such compounds that affect another step in the SCF/c-Kit signal transduction pathway in which the c-Kit gene and/or c-Kit gene product is involved and, by affecting this same pathway may modulate the effect of c-Kit on the modulation of angiogenesis are within the scope of the invention. Such compounds can be used as part of a therapeutic method to modulate angiogenesis.

The invention encompasses cell-based and animal model-based assays for the identification of compounds exhibiting such an ability to modulate angiogenesis. Such cell-based assay systems can also be used as the “gold standard” to assay for purity and potency of the natural ligand, SCF, including recombinantly or synthetically produced SCF and SCF mutants.

Cell-based systems, membrane vesicle-based systems and membrane fraction-based systems can be used to identify compounds that may act to modulate angiogenesis. Such cell systems can include, for example, recombinant or non-recombinant cells, such as cell lines, that express the SCF or c-Kit gene. In addition, expression host cells (e.g., COS cells, CHO cells, fibroblasts) genetically engineered to express a functional c-Kit and to respond to activation by the natural SCF ligand, e.g., as measured by a chemical or phenotypic change, induction of another host cell gene, change in ion flux (e.g., Ca⁺⁺), tyrosine phosphorylation of host cell proteins, etc., can be used as an end point in the assay.

In utilizing such cell systems, cells may be exposed to a compound suspected of exhibiting an ability to modulate angiogenesis, at a sufficient concentration and for a time sufficient to elicit such modulation of angiogenesis as measured by a chemical or phenotypic change in the exposed cells. After exposure, the cells can be assayed to measure alterations in the expression of the SCF or c-Kit genes, e.g., by assaying cell lysates for SCF or c-Kit mRNA transcripts (e.g., by Northern analysis) or for SCF or c-Kit protein expressed in the cell; compounds that regulate or modulate expression of the SCF or c-Kit genes are good candidates as therapeutics. Alternatively, the cells are examined to determine whether angiogenesis has been modulated as measured by a chemical or phenotypic change. Still further, the expression and/or activity of components of the signal transduction pathway of which SCF/c-Kit are a part, or the activity of the SCF/c-Kit signal transduction pathway itself can be assayed.

For example, after exposure, the cell lysates can be assayed for the presence of tyrosine phosphorylation of host cell proteins, as compared to lysates derived from unexposed control cells. The ability of a test compound to inhibit tyrosine phosphorylation of host cell proteins in these assay systems indicates that the test compound inhibits signal transduction initiated by c-Kit activation. The cell lysates can be readily assayed using a Western blot format; i.e., the host cell proteins are resolved by gel electrophoresis, transferred and probed using a anti-phosphotyrosine detection antibody (e.g., an anti-phosphotyrosine antibody labeled with a signal generating compound, such as radiolabel, fluor, enzyme, etc.) (See, e.g., Glenney et al. 1988 J Immunol Methods 109:277-285; Frackelton et al. 1983 Mol Cell Biol 3:1343-1352). Alternatively, an ELISA format could be used in which a particular host cell protein involved in the SCF/c-Kit signal transduction pathway is immobilized using an anchoring antibody specific for the target host cell protein, and the presence or absence of phosphotyrosine on the immobilized host cell protein is detected using a labeled anti-phosphotyrosine antibody. (See, King et al. 1993 Life Sciences 53:1465-1472). In yet another approach, ion flux, such as calcium ion flux, can be measured as an end point for SCF/c-Kit stimulated signal transduction.

In addition, animal-based models of angiogenesis may be used to identify compounds capable of modulating angiogenesis. Such animal models may be used as test substrates for the identification of drugs, pharmaceuticals, therapies and interventions that may be effective in treating conditions where modulation of angiogenesis is desired. For example, animal models may be exposed to a compound, suspected of exhibiting an ability to modulate angiogenesis, at a sufficient concentration and for a time sufficient to elicit such a modulation of angiogenesis in the exposed animals. The response of the animals to the exposure may be monitored by assessing phenotypic correlates associated with angiogenesis. With regard to intervention, any treatments that modulate angiogenesis or phenotypic correlates of angiogenesis should be considered as candidates for therapeutic intervention. Dosages of test agents may be determined by deriving dose-response curves.

Inhibition of SCF or c-Kit Expression or SCF or c-Kit Activity to Inhibit Angiogenesis

Any method that neutralizes SCF or inhibits expression of the c-Kit gene (either transcription or translation) can be used to modulate angiogenesis. Such approaches can be used to promote angiogenesis or to inhibit angiogenesis.

For example, the administration of soluble peptides, proteins, fusion proteins, or antibodies (including anti-idiotypic antibodies) that bind to and “neutralize” circulating SCF, the natural ligand for c-Kit, can be used to inhibit angiogenesis. To this end, peptides corresponding to the ECD of c-Kit, soluble deletion mutants of c-Kit (e.g., a deletion mutant in which the TM domain is deleted), or either of these c-Kit domains or mutants fused to another polypeptide (e.g., an IgFc polypeptide) can be utilized. Alternatively, anti-idiotypic antibodies or Fab fragments of antiidiotypic antibodies that mimic the c-Kit ECD and neutralize SCF can be used. Such c-Kit peptides, proteins, fusion proteins, anti-idiotypic antibodies or Fabs are administered to a subject in amounts sufficient to neutralize SCF and to inhibit angiogenesis.

In an alternative embodiment for neutralizing circulating SCF, cells that are genetically engineered to express such soluble or secreted forms of c-Kit may be administered to a patient, whereupon they will serve as “bioreactors” in vivo to provide a continuous supply of the SCF neutralizing protein. Such cells may be obtained from the patient or an MHC compatible donor and can include, but are not limited to fibroblasts, blood cells (e.g., lymphocytes), adipocytes, muscle cells, endothelial cells etc. The cells are genetically engineered in vitro using recombinant DNA techniques to introduce the coding sequence for the c-Kit ECD, a deletion mutant in which the TM domain of c-Kit is deleted, or for c-Kit-Ig fusion protein (e.g., c-Kit-, ECD- or c-Kit TM domain deletion-IgFc fusion proteins) into the cells, e.g., by transduction (using viral vectors, and preferably vectors that integrate the transgene into the cell genome) or transfection procedures, including but not limited to the use of plasmids, cosmids, YACs, electroporation, liposomes, etc. The c-Kit coding sequence can be placed under the control of a strong constitutive or inducible promoter or promoter/enhancer to achieve expression and secretion of the c-Kit peptide or fusion protein. The engineered cells which express and secrete the desired c-Kit product can be introduced into the patient systemically. Alternatively, the cells can be incorporated into a matrix and implanted in the body, e.g., genetically engineered fibroblasts can be implanted as part of a skin graft; genetically engineered endothelial cells can be implanted as part of a vascular graft. (See, for example, Anderson et al. U.S. Pat. No. 5,399,349; and Mulligan & Wilson, U.S. Pat. No. 5,460,959.)

When the cells to be administered are non-autologous cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Antisense RNA

Antisense RNA Genes may be constructed or isolated that produce antisense RNA that is complementary to all or part(s) of a targeted messenger RNA(s). The antisense RNA reduces production of the polypeptide product of the messenger RNA. The polypeptide product may be any protein encoded by the genome. The aforementioned genes will be referred to as antisense genes. An antisense gene may be introduced into a tissue culture cell line or individual by transformation methods to reduce expression of a selected protein of interest. Construction of genes encoding antisense RNAs is well within the skill of the ordinary practitioner, once a suitable target sequence is known.

The term “antisense construct” is intended to refer to nucleic acids, preferably oligonucleotides, that are complementary to the base sequences of a target DNA or RNA. Targeting double-stranded (ds) DNA with an antisense construct leads to triple-helix formation. Targeting RNA will lead to double-helix formation. Antisense nucleic acids, when introduced into a target cell, specifically bind to their target polynucleotide and interfere with transcription, RNA processing, RNA transport, translation and/or stability. Antisense RNA constructs, or DNA encoding such antisense RNAs, may be employed to inhibit gene transcription or translation or both within cells.

Antisense constructs may be designed to bind to complementary sequences within the promoter region or other control regions, exons, introns or even exon-intron boundaries of a gene. Antisense RNA constructs, or DNA encoding such antisense RNAs, may be employed to inhibit gene transcription or translation or both within a host cell, either in vitro or in vivo, such as within a human subject. Nucleic acid sequences which comprise “complementary sequences” are those which are capable of base-pairing according to the standard Watson-Crick complementarity rules. Inclusion of less common bases such as inosine, 5-methylcytosine, 6-methyladenine, hypoxanthine and others in hybridizing sequences does not interfere with base-pairing.

As used herein, the term “complementary” means nucleic acid sequences that are substantially complementary over their entire length and have very few base mismatches. For example, nucleic acid sequences of fifteen bases in length may be termed complementary when they have a complementary nucleotide at thirteen or fourteen positions with only a single mismatch. Naturally, nucleic acid sequences which are “completely complementary” will be nucleic acid sequences which are entirely complementary throughout their entire length and have no base mismatches.

Other sequences with lower degrees of homology also are contemplated. For example, an antisense construct that has limited regions of high homology, but also contains a non-homologous region (e.g., a ribozyme) could be designed. These molecules, though having less than 50% homology, would bind to target sequences under appropriate conditions.

While all or part of the gene sequences may be employed in the context of antisense construction, short oligonucleotides are easier to make and increase in vivo accessibility. However, both binding affinity and sequence specificity of an antisense oligonucleotide to its complementary target increases with increasing length. One can readily determine whether a given antisense nucleic acid is effective at targeting of the corresponding host cell gene by testing the constructs in vitro to determine whether the function of the endogenous gene is affected or whether the expression of related genes having complementary sequences is affected.

In certain embodiments, one may wish to employ antisense constructs that include other elements, for example, those which include C-5 propyne pyrimidines. Oligonucleotides that contain C-5 propyne analogues of uridine and cytidine have been shown to bind RNA with high affinity and to be potent antisense inhibitors of gene expression.

Ribozymes

Another method for inhibiting target gene expression contemplated in the present invention is via ribozymes. Ribozymes are RNA-protein complexes that cleave nucleic acids in a site-specific fashion. Ribozymes have specific catalytic domains that possess endonuclease activity. A large number of ribozymes accelerate phosphoester transfer reactions with a high degree of specificity, often cleaving only one of several phosphoesters in an oligonucleotide substrate. This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids. For example, investigators report that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications. It has been reported that ribozymes elicit genetic changes in some cells lines to which they were applied. The altered genes included the oncogenes H-ras, c-fos and genes of HIV. Most of this work involved the modification of a target mRNA, based on a specific mutant codon that is cleaved by a specific ribozyme.

Several different ribozyme motifs have been described with RNA cleavage activity. Examples that are expected to function equivalently for the down regulation of genes include sequences from the Group I self splicing introns. These include Tobacco Ringspot Virus, Avocado Sunblotch Viroid, and Lucerne Transient Streak Virus. Sequences from these and related viruses are referred to as hammerhead ribozymes based on a predicted folded secondary structure.

Other suitable ribozymes include sequences from RNase P, hairpin ribozyme structures and Hepatitis Delta virus based ribozymes. The general design and optimization of ribozyme directed RNA cleavage activity has been discussed in detail in the literature.

The other variable on ribozyme design is the selection of a cleavage site on a given target RNA. Ribozymes are targeted to a given sequence by virtue of annealing to a site by complementary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence that is the cleavage site. For hammerhead ribozymes, the cleavage site is a dinucleotide sequence on the target RNA—a uracil (U) followed by either an adenine, cytosine or uracil (A, C or U). The frequency of this dinucleotide occurring in any given RNA is statistically 3 out of 16. Designing and testing ribozymes for efficient cleavage of a target RNA is a process well known to those skilled in the art.

RNA Interference

RNA interference refers to the process of sequence-specific post-transcriptional gene silencing in animals mediated by short interfering RNAs (siRNAs) (Fire et al. 1998 Nature 391:806-811). The corresponding process in plants is commonly referred to as post-transcriptional gene silencing or RNA silencing and is also referred to as quelling in fungi. The process of post-transcriptional gene silencing is thought to be an evolutionarily-conserved cellular defense mechanism used to prevent the expression of foreign genes which is commonly shared by diverse flora and phyla (Fire et al. 1999 Trends Genet 15:358-363). Such protection from foreign gene expression may have evolved in response to the production of double-stranded RNAs (dsRNAs) derived from viral infection or from the random integration of transposon elements into a host genome via a cellular response that specifically destroys homologous single-stranded RNA or viral genomic RNA. The presence of dsRNA in cells triggers the RNAi response through a mechanism that has yet to be fully characterized. This mechanism appears to be different from the interferon response that results from dsRNA-mediated activation of protein kinase PKR and 2′,5′-oligoadenylate synthetase resulting in non-specific cleavage of mRNA by ribonuclease L.

The presence of long dsRNAs in cells stimulates the activity of a ribonuclease III enzyme referred to as dicer. Dicer is involved in the processing of the dsRNA into short pieces of dsRNA known as short interfering RNAs (siRNAs) (Bernstein et al. 2001 Nature 409:363-366). Short interfering RNAs derived from dicer activity are typically about 21-23 nucleotides in length and comprise about 19 base pair duplexes. Dicer has also been implicated in the excision of 21 and 22 nucleotide small temporal RNAs (stRNAs) from precursor RNA of conserved structure that are implicated in translational control (Hutvagner et al. 2001 Science 293:834-838). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al. 2001 Genes Dev 15:188-200).

Short interfering RNA mediated RNAi has been studied in a variety of systems. Fire et al. 1998 Nature 391:806-811, were the first to observe RNAi in C. elegans. Wianny, F. and Zernicka-Goetz, M. 1999 Nature Cell Biol 2:70-75, describe RNAi mediated by dsRNA in mouse embryos. Hammond et al. 2000 Nature 404:293-296, describe RNAi in Drosophila cells transfected with dsRNA. Elbashir et al. 2001 Nature 411:494-498, describe RNAi induced by introduction of duplexes of synthetic 21-nucleotide RNAs in cultured mammalian cells including human embryonic kidney and HeLa cells. Recent work in Drosophila embryonic lysates (Elbashir et al. 2001 EMBO J 20:6877-6888) has revealed certain requirements for siRNA length, structure, chemical composition, and sequence that are essential to mediate efficient RNAi activity. These studies have shown that 21-nucleotide siRNA duplexes are most active when containing 3′-terminal di-nucleotide overhangs. Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the siRNA guide sequence (Elbashir et al. 2001 EMBO J 20:6877-6888). Other studies have indicated that a 5′-phosphate on the target-complementary strand of a siRNA duplex is required for siRNA activity and that ATP is utilized to maintain the 5′-phosphate moiety on the siRNA (Nykanen et al. 2001 Cell 107:309-321).

Restoration or Increase in SCF or c-Kit Expression or Activity to Promote Angiogenesis

With respect to an increase in the level of normal SCF or c-Kit gene expression and/or SCF or c-Kit gene product activity, SCF or c-Kit nucleic acid sequences can be utilized to promote angiogenesis. Where a deficiency in angiogenesis is due to a defective SCF or c-Kit, treatment can be administered, for example, in the form of gene replacement therapy. Specifically, one or more copies of a normal SCF or c-Kit gene or a portion of the SCF or c-Kit genes that direct the production of an SCF or c-Kit gene product exhibiting normal function, may be inserted into the appropriate cells within a patient or animal subject, using vectors which include, but are not limited to adenovirus, adeno-associated virus, retrovirus, lentivirus and herpes virus vectors, in addition to other particles that introduce DNA into cells, such as liposomes.

Because the SCF and c-Kit genes are expressed, among other areas, in the brain, such gene replacement therapy techniques should be capable of delivering SCF or c-Kit gene sequences to these cell types within patients. Thus, the techniques for delivery of the SCF or c-Kit gene sequences should be designed to readily cross the blood-brain barrier, which are well known to those of skill in the art (see, e.g., PCT application, publication No. WO89/10134), or, alternatively, should involve direct administration of such SCF or c-Kit gene sequences to the site of the cells in which the SCF or c-Kit gene sequences are to be expressed. Alternatively, targeted homologous recombination can be utilized to correct the defective endogenous SCF or c-Kit genes in appropriate tissues.

Additional methods that may be utilized to increase the overall level of SCF or c-Kit gene expression and/or SCF or c-Kit activity include the introduction of appropriate SCF- or c-Kit-expressing cells, preferably autologous cells, into a patient at positions and in numbers that are sufficient to promote angiogenesis. Such cells may be either recombinant or non-recombinant. Among the cells that can be administered to increase the overall level of SCF or c-Kit gene expression in a patient are normal cells that express the SCF or c-Kit genes. The cells can be administered at the anatomical site in the brain, or as part of a tissue graft located at a different site in the body. Such cell-based gene therapy techniques are well known to those skilled in the art, see, e.g., Anderson, et al., U.S. Pat. No. 5,399,349; Mulligan & Wilson, U.S. Pat. No. 5,460,959.

Finally, compounds, identified in the assays described above, that stimulate or enhance the signal transduced by activated SCF/c-Kit, e.g., by activating downstream signaling proteins in the SCF/c-Kit cascade and thereby by-passing the defective SCF or c-Kit, can be used to promote angiogenesis. The formulation and mode of administration will depend upon the physico-chemical properties of the compound. The administration should include known techniques that allow for a crossing of the blood-brain barrier.

Modulators

The compounds tested as modulators of a SCF or c-Kit family member can be any small chemical compound, or a biological entity, such as a protein, sugar, nucleic acid or lipid. Alternatively, modulators can be genetically altered versions of a SCF or c-Kit gene. Typically, test compounds will be small chemical molecules and peptides.

Essentially any chemical compound can be used as a potential modulator or ligand in the assays of the invention, although most often compounds dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In one convenient embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particularly chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175; Furka 1991 Int J Pept Prot Res 37:487-493 and Houghton et al. 1991 Nature 354:84-88). Other chemistries for generating chemical diversity libraries can also be used. Such chemistries include, but are not limited to: peptides (e.g., PCT Publication No. WO 91/19735), encoded peptides (e.g., PCT Publication WO 93/20242), random bio-oligomers (e.g., PCT Publication No. WO 10 92/00091), benzodiazepines (e.g., U.S. Pat. No. 5,288,514), diversomers such as hydantoins, benzodiazepines and dipeptides (Hobbs et al. 1993 PNAS USA 90:6909-6913), vinylogous polypeptides (Hagihara et al. 1992 J Amer Chem Soc 114:6568), nonpeptidal peptidomimetics with glucose scaffolding (Hirschmann et al. 1992 J Amer Chem Soc 114:9217-9218), analogous organic syntheses of small compound libraries (Chen et al. 15 1994 J Amer Chem Soc 116:2661), oligocarbamates (Cho et al. 1993 Science 261:1303), and/or peptidyl phosphonates (Campbell et al. 1994 J Org Chem 59:658), nucleic acid libraries (see Sambrook et al. 1989 in Molecular Cloning, A Laboratorv Manual, Cold Springs Harbor Press, N.Y.; and Ausubel et al. 1989 in Current Protocols in Molecular Biology Green Publishing Associates and Wiley Interscience, N.Y.), peptide nucleic acid libraries (see, e.g., U.S. Pat. No. 5,539,083), antibody libraries (see, e.g., Vaughn et al. 1996 Nature Biotechnology 14:309-314 and PCT/US96/10287), carbohydrate libraries (see, e.g., Liang et al. 1996 Science 274:1520-1522 and U.S. Pat. No. 5,593,853), small organic molecule libraries (see, e.g., benzodiazepines, Baum C & EN, Jan. 18, 1993 page 33; isoprenoids, U.S. Pat. No. 5,569,588; thiazolidionones and methathiazones, U.S. Pat. No. 255,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville, Ky.; Symphony, Rainin, Woburn, Mass.; 433A Applied Biosystems, Foster City, Calif.; 9050 Plus, Millipore, Bedford, Mass.). In addition, numerous combinatorial libraries are themselves commercially available (see, e.g., ComGenex, Princeton, N.J.; Tripos, Inc., St. Louis, Mo.; 3D Pharmaceuticals, Exton, Pa.; Martek Biosciences, Columbia, Md.; etc.).

Solid State and Soluble High Throughput Assays

In one embodiment the invention provides soluble assays using molecules such as a domain such as a ligand binding domain, an extracellular domain, a transmembrane domain, a transmembrane domain and a cytoplasmic domain, an active site, a subunit association region, etc.; a domain that is covalently linked to a heterologous protein to create a chimeric molecule; an SCF or c-Kit receptor protein; or a cell or tissue expressing an SCF or c-Kit receptor protein, either naturally occurring or recombinant. In another embodiment, the invention provides solid phase based in vitro assays in a high throughput format, where the domain, chimeric molecule, SCF or c-Kit receptor protein, or cell or tissue expressing the SCF or c-Kit receptor is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay several different plates per day; assay screens for up to about 6,000-20,000 different compounds are possible using the integrated systems of the invention. More recently, microfluidic approaches to reagent manipulation have been developed.

The molecule of interest can be bound to the solid state component, directly or indirectly, via covalent or non-covalent linkage, e.g., via a tag. The tag can be any of a variety of components. In general, a molecule which binds the tag (a tag binder) is fixed to a solid support, and the tagged molecule of interest (e.g., SCF or c-Kit) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and appropriate tag binders; see, SIGMA Immunochemicals 1998 catalogue SIGMA, St. Louis, Mo.

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody that recognizes the first antibody. In addition to antibody-antigen interactions, receptor-ligand interactions are also appropriate as tag and tag-binder pairs.

For example, agonists and antagonists of cell membrane receptors (e.g., cell receptor-ligand interactions such as transferrin, viral receptor ligands, cytokine receptors, chemokine receptors, interleukin receptors, immunoglobulin receptors and antibodies, the cadherein family, the integrin family, the selectin family, and the like; (see, e.g., Pigott & Power 1993 in The Adhesion Molecule Facts Book I). Similarly, toxins and venoms, viral epitopes, hormones (e.g., opiates, steroids, etc. ), intracellular receptors (e.g., which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethelyne glycol) linkers are available from Shearwater Polymers, Inc. Huntsville, Alabama. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent that fixes a chemical group to the surface that is reactive with a portion of the tag binder. For example, groups that are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield 1963 J Am Chem Soc 85.2149-2154 (describing solid phase synthesis of, e.g., peptides); Geysen et al. 1987 J Immun Meth 102:259-274 (describing synthesis of solid phase components on pins); Frank & Doring 1988 Tetrahedron 44:6031-6040 (describing synthesis of various peptide sequences on cellulose disks); Fodor et al. 1991 Science 251:767-777; Sheldon et al. 1993 Clinical Chemistry 39:718-719; and Kozal et al. 1996 Nature Medicine 2:753759 (all describing arrays of biopolymers fixed to solid substrates). Non-chemical approaches for fixing tag binders to substrates include other common methods, such as heat, cross-linking by UV radiation, and the like.

Computer-Based Assays

Yet another assay for compounds that modulate SCF or c-Kit receptor protein activity involves computer assisted drug design, in which a computer system is used to generate a three-dimensional structure of an SCF or c-Kit receptor protein based on the structural information encoded by its amino acid sequence. The input amino acid sequence interacts directly and actively with a preestablished algorithm in a computer program to yield secondary, tertiary, and quaternary structural models of the protein. The models of the protein structure are then examined to identify regions of the structure that have the ability to bind, e.g., ligands. These regions are then used to identify ligands that bind to the protein. The three-dimensional structural model of the protein is generated by entering protein amino acid sequences of at least 10 amino acid residues or corresponding nucleic acid sequences encoding a SCF or c-Kit polypeptide into the computer system. The nucleotide sequence encoding the polypeptide, or the amino acid sequence thereof, can be any of any “wild-type” and “mutant” SCF or c-Kit receptor. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Walls potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of the SCF or c-Kit receptor protein to identify ligands that bind to the protein. Binding affinity between the protein and ligands is determined using energy terms to determine which ligands have an enhanced probability of binding to the protein.

Determining Polypeptide and Polynucleofide Identity and Similarity

Preferred identity and/or similarity are designed to give the largest match between the sequences tested. Methods to determine identity and similarity are codified in computer programs including, but are not limited to, the GCG program package, including GAP (Devereux, J., et al., 1984 Nucleic Acids Research 12:387-95; Genetics Computer Group, University of Wisconsin, Madison, Wis.), BLASTP, BLASTN, BLASTX, FASTA (Altschul, S. F. et al. 1990, J. Molec. Biol. 215:403-410, PSI-BLAST (Altschul S. F. et al. 1997 Nucleic Acids Res. 25:3389-3402), the eMatrix software (Wu et al. 1999 J. Comp. Biol. 6:219-235), eMotif software (Nevill-Manning et al. 1997 Proc Int Conf Intell Syst Mol Biol 5:202-209), the GeneAtlas software (Molecular Simulations Inc. (MSI), San Diego, Calif.) (Sanchez and Safl 1998 Proc. Natl. Acad. Sci. 95:13597-13602; Fischer and Eisenberg 1996 Protein Sci. 5:947-955), Neural Network SignalP V1.1 program (from Center for Biological Sequence Analysis, The Technical University of Denmark) and the Kyte-Doolittle hydrophobicity prediction algorithm (Kyte and Doolittle 1982 J. Mol Biol 157:105-31). The BLAST programs are publicly available from the National Center for Biotechnology Information (NCBI) and other sources (BLAST Manual, Altschul, S., et al. NCBI NLM NIFI Bethesda, Md. 20894; Altschul, S., et al. 1990 J. Mol. Biol. 215:403-410.

Gene Therapy

The invention provides gene therapy to mimic normal activity of the polypeptides of the invention; or to treat disease states involving polypeptides of the invention. Delivery of a functional gene encoding polypeptides of the invention to appropriate cells is effected ex vivo, in situ, or in vivo by use of vectors, and more particularly viral vectors (e.g., adenovirus, adeno-associated virus, or a retrovirus), or ex vivo by use of physical DNA transfer methods (e.g., liposomes or chemical treatments). See, for example, Anderson 1998 Nature suppl 392:25-30. For additional reviews of gene therapy technology see Friedmann 1989 Science 244:1275-1281; Verma 1990 Scientific American 263:68-72, 81-84; and Miller 1992 Nature 357:455-460. Introduction of any one of the nucleotides of the present invention or a gene encoding the polypeptides of the present invention can also be accomplished with extrachromosomal substrates (transient expression) or artificial chromosomes (stable expression).

Uses and Biological Activity of Human SCF, SCF-Like, c-Kit or c-Kit-Like Polypeptides

The polynucleotides and proteins of the present invention are expected to exhibit one or more of the uses or biological activities identified herein. Uses or activities described for proteins of the present invention may be provided by administration or use of such proteins or of polynucleotides encoding such proteins (such as, for example, in gene therapies or vectors suitable for introduction of DNA). The mechanism underlying the particular condition or pathology will dictate whether the polypeptides of the invention, the polynucleotides of the invention, or modulators (activators or inhibitors) thereof would be beneficial to the subject in need of treatment. Thus, “therapeutic compositions of the invention” include compositions comprising isolated polynucleotides (including recombinant DNA molecules, cloned genes and degenerate variants thereof), polypeptides of the invention (including full length protein, mature protein and truncations or domains thereof), or compounds and other substances that modulate the overall activity of the target gene products, either at the level of target gene/protein expression or target protein activity.

Chemotactic/Chemokinetic Activity

A polypeptide of the present invention may be involved in chemotactic or chemokinetic activity for mammalian cells, including, for example, neural stem, progenitor, and precursor cells. A polynucleotide of the invention can encode a polypeptide exhibiting such attributes. Chemotactic and chemokinetic receptor activation can be used to mobilize or attract a desired cell population to a desired site of action. Chemotactic or chemokinetic compositions (e.g. proteins or peptides) provide particular advantages in treatment of lesions of the central nervous system. For example, attraction of neural stem, progenitor or precursor cells to sites of lesions may result in improved central nervous system function.

A SCF or SCF-like protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

Therapeutic compositions of the invention can be used in the following.

Assays for chemotactic activity (that will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Coligan et al 1991 Current Protocols in Immunology, Pub. Greene Publishing Associates and Wiley-Interscience (Chapter 6.12, Measurement of alpha and beta Chemokines 6.12.1-6.12.28); Taub et al 1995. J. Clin. Invest. 95:1370-1376; Lind et al. 1995 APMIS 103:140-146; Muller et al 1995 Eur. J. Immunol. 25:1744-1748; Gruber et al. 1994 J. of Immunol. 152:5860-5867; Johnston et al. 1994 J. of Immunol. 153:1762-1768.

Nervous System Disorders

Nervous system disorders that can be treated include but are not limited to nervous system injuries, and diseases or disorders that result in either a disconnection of axons, a diminution or degeneration of neurons, or demyelination. Nervous system lesions that may be treated in a patient (including human and non-human mammalian patients) according to the invention include but are not limited to the following lesions of the central (including spinal cord, brain) nervous system:

(i) traumatic lesions, including lesions caused by physical injury or associated with surgery, for example, lesions that sever a portion of the nervous system, or compression injuries;

(ii) ischemic lesions, in which a lack of oxygen in a portion of the nervous system results in neuronal injury or death, including cerebral infarction or ischemia, or spinal cord infarction or ischemia;

(iii) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis;

(iv) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis;

(v) malignant lesions, in which a portion of the nervous system is destroyed or injured as a result of a malignancy;

(vi) lesions associated with nutritional diseases or disorders, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration;

(vii) neurological lesions associated with systemic diseases including but not limited to diabetes (diabetic neuropathy, Bell's palsy), systemic lupus erythematosus, carcinoma, or sarcoidosis;

(viii) lesions caused by toxic substances including alcohol, lead, or particular neurotoxins; and

(ix) demyelinated lesions in which a portion of the nervous system is destroyed or injured by a demyelinating disease including but not limited to multiple sclerosis, human immunodeficiency virus-associated myelopathy, transverse myelopathy or various etiologies, progressive multifocal leukoencephalopathy, and central pontine myelinolysis.

Therapeutic Methods

The compositions (including polypeptides and fragments, analogs, and variants thereof) of the invention have numerous applications in a variety of therapeutic methods. Examples of therapeutic applications include, but are not limited to, those exemplified herein.

Pharmaceutical Formulations and Routes of Administration

A protein or other composition of the present invention (from whatever source derived, including without limitation from recombinant and non-recombinant sources) may be administered to a patient in need, by itself, or in pharmaceutical compositions where it is mixed with suitable carriers or excipient(s) at doses to treat or ameliorate a variety of disorders. Such a composition may optionally contain (in addition to protein or other active ingredient and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNFO, TNF1, TNF2, G-CSF, Meg-CSF, thrombopoietin, and erythropoietin. In further compositions, proteins of the invention may be combined with other agents beneficial to the treatment of the disease or disorder in question. These agents include various growth factors such as epidermal growth factor (EGF), platelet-derived growth factor (PDGF), transforming growth factors (TGF-α and TGF-β), insulin-like growth factor (IGF), as well as cytokines described herein.

The pharmaceutical composition may farther contain other agents that either enhance the activity of the protein or other active ingredient or complement its activity or use in treatment. Such additional factors and/or agents may be included in the pharmaceutical composition to produce a synergistic effect with protein or other active ingredient of the invention, or to minimize side effects. Conversely, protein or other active ingredient of the present invention may be included in formulations of the particular cytokine to minimize side effects of the cytokine (such as IL-1Ra, IL-1 Hy1, IL-1 Hy2, anti-TNF, corticosteroids, immunosuppressive agents). A protein of the present invention may be active in multimers (e.g., heterodimers or homodimers) or complexes with itself or other proteins. As a result, pharmaceutical compositions of the invention may comprise a protein of the invention in such multimeric or complexed form.

As an alternative to being included in a pharmaceutical composition of the invention including a first protein, a second protein or a therapeutic agent may be concurrently administered with the first protein (e.g., at the same time, or at differing times provided that therapeutic concentrations of the combination of agents is achieved at the treatment site). Techniques for formulation and administration of the compounds of the instant application may be found in “Remington's Pharmaceutical Sciences” Mack Publishing Co., Easton, Pa., 1985. A therapeutically effective dose further refers to that amount of the compound sufficient to result in amelioration of symptoms, e.g., treatment, healing, prevention or amelioration of the relevant medical condition, or an increase in rate of treatment, healing, prevention or amelioration of such conditions. When applied to an individual active ingredient, administered alone, a therapeutically effective dose refers to that ingredient alone. When applied to a combination, a therapeutically effective dose refers to combined amounts of the active ingredients that result in the therapeutic effect, whether administered in combination, serially or simultaneously.

In practicing the method of treatment or use of the present invention, a therapeutically effective amount of protein or other active ingredient of the present invention is administered to a mammal having a condition to be treated. Protein or other active ingredient of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein or other active ingredient of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein or other active ingredient of the present invention in combination with cytokine(s), lymphokine(s), other hematopoietic factor(s), thrombolytic or anti-thrombotic factors.

Routes of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Administration of protein or other active ingredient of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection.

Alternately, one may administer the compound in a local rather than systemic manner, for example, via injection of the compound directly into tissue, often in a depot or sustained release formulation.

The polypeptides of the invention are administered by any route that delivers an effective dosage to the desired site of action. The determination of a suitable route of administration and an effective dosage for a particular indication is within the level of skill in the art. Preferably for wound treatment, one administers the therapeutic compound directly to the site. Suitable dosage ranges for the polypeptides of the invention can be extrapolated from these dosages or from similar studies in appropriate animal models. Dosages can then be adjusted as necessary by the clinician to provide maximal therapeutic benefit.

Compositions/Formulations

Pharmaceutical compositions for use in accordance with the present invention thus may be formulated in a conventional manner using one or more physiologically acceptable carriers comprising excipients and auxiliaries that facilitate processing of the active compounds into preparations that can be used pharmaceutically. These pharmaceutical compositions may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen. When a therapeutically effective amount of protein or other active ingredient of the present invention is administered orally, protein or other active ingredient of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein or other active ingredient of the present invention, and preferably from about 25 to 90% protein or other active ingredient of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may farther contain physiological saline solution, dextrose or other saccharide solution, or glycols such as ethylene glycol, propylene glycol or polyethylene glycol. When administered in liquid form, the pharmaceutical composition contains from. about 0.5 to 90% by weight of protein or other active ingredient of the present invention, and preferably from about 1 to 50% protein or other active ingredient of the present invention.

When a therapeutically effective amount of protein or other active ingredient of the present invention is administered by intravenous, cutaneous or subcutaneous injection, protein or other active ingredient of the present invention will be in the form of a pyrogen-free, parenterally acceptable aqueous solution. The preparation of such parenterally acceptable protein or other active ingredient solutions, having due regard to pH, isotonicity, stability, and the like, is within the skill in the art. A preferred pharmaceutical composition for intravenous, cutaneous, or subcutaneous injection should contain, in addition to protein or other active ingredient of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Dragee cores are provided with suitable coatings. For this purpose, concentrated sugar solutions may be used that may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alliteratively, the active ingredient may be in powder form for constitution with a suitable vehicle, e.g., sterile pyrogen-free water, before use.

In addition to the formulations described previously, the compounds may also be formulated as a depot preparation. Such long acting formulations may be administered by implantation or by injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. The co-solvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (V-PD: 5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may substitute for dextrose. Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various types of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein or other active ingredient stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols. Many of the active ingredients of the invention may be provided as salts with pharmaceutically compatible counter ions. Such pharmaceutically acceptable base addition salts are those salts that retain the biological effectiveness and properties of the free acids and that are obtained by reaction with inorganic or organic bases such as sodium hydroxide, magnesium hydroxide, ammonia, trialkylamine, dialkylamine, monoalkylamine, dibasic amino acids, sodium acetate, potassium benzoate, triethanol amine and the like.

The pharmaceutical composition of the invention may be in the form of a liposome in which protein of the present invention is combined, in addition to other pharmaceutically acceptable carriers, with amphipathic, agents such as lipids that exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithins, phospholipids, saponin, bile acids, and the like. Preparation of such liposomal formulations is within the level of skill in the art, as disclosed, for example, in U.S. Pat. Nos. 4,235,871; 4,501,728; 4,837,028; and 4,737,323.

The amount of protein or other active ingredient of the present invention in the pharmaceutical composition of the present invention will depend upon the nature and severity of the condition being treated, and on the nature of prior treatments that the patient has undergone. Ultimately, the attending physician will decide the amount of protein or other active ingredient of the present invention with which to treat each individual patient. Initially, the attending physician will administer low doses of protein or other active ingredient of the present invention and observe the patient's response. Larger doses of protein or other active ingredient of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 μg to about 10 mg, more preferably about 0.1 μg to about 1 mg) of protein or other active ingredient of the present invention per kg body weight. For compositions of the present invention that are useful for tissue regeneration, the therapeutic method includes administering the composition topically, systematically, or locally as an implant or device. When administered, the therapeutic composition for use in this invention is, of course, in a pyrogen-free, physiologically acceptable form. Further, the composition may desirably be encapsulated or injected in a viscous form for delivery to the site of tissue damage. Therapeutically useful agents other than a protein or other active ingredient of the invention that may also optionally be included in the composition as described above, may alternatively or additionally, be administered simultaneously or sequentially with the composition in the methods of the invention.

The therapeutic compositions are also presently valuable for veterinary applications. Particularly domestic animals and thoroughbred horses, in addition to humans, are desired patients for such treatment with proteins or other active ingredient of the present invention. The dosage regimen of a protein-containing pharmaceutical composition to be used in tissue regeneration will be determined by the attending physician considering various factors that modify the action of the proteins, e.g., amount of tissue weight desired to be formed, the site of damage, the condition of the damaged tissue, the size of a wound, type of damaged tissue, the patient's age, sex, and diet, the severity of any infection, time of administration and other clinical factors. Progress can be monitored by periodic assessment of tissue growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism. (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amount is well within the capability of those skilled in the art, especially in light of the detailed disclosure provided herein. For any compound used in the method of the invention, the therapeutically effective dose can be estimated initially from appropriate in vitro assays. For example, a dose can be formulated in animal models to achieve a circulating concentration range that can be used to more accurately determine useful doses in humans. For example, a dose can be formulated in animal models to achieve a circulating concentration range that includes the IC₅₀ as determined in cell culture (i.e., the concentration of the test compound that achieves a half-maximal inhibition of the protein's biological activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀, (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al. 1975 in The Pharmacological Basis of Therapeutics, Ch. 1 p. 1. Dosage amount and interval may be adjusted individually to provide plasma levels of the active moiety that are sufficient to maintain the desired effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen that maintains plasma levels above the MEC for 10-90% of the time, preferably between 30-90% and most preferably between 50-90%. In cases of local administration or selective uptake, the effective local concentration of the drug may not be related to plasma concentration.

An exemplary dosage regimen for polypeptides or other compositions of the invention will be in the range of about 0.01 μg/kg to 100 mg/kg of body weight daily, with the preferred dose being about 0.1 82 g/kg to 25 mg/kg of patient body weight daily, varying in adults and children. Dosing may be once daily, or equivalent doses may be delivered at longer or shorter intervals.

The amount of composition administered will, of course, be dependent on the subject being treated, on the subject's age and weight, the severity of the affliction, the manner of administration and the judgment of the prescribing physician.

Packaging

The compositions may, if desired, be presented in a pack or dispenser device that may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

Neuronal and Glioma-Derived Stem Cell Factor Induces Angiogenesis Within the Brain

Stem cell factor (SCF) is overexpressed by neurons following brain injury as well as by glioma cells; however, its role in gliomagenesis remains unclear. Here, we demonstrate that SCF directly activates brain microvascular endothelial cells (ECs) in vitro and induces a potent angiogenic response in vivo. Primary human gliomas express SCF in a grade-dependent manner and induce normal neurons to express SCF in brain regions infiltrated by glioma cells, areas that colocalize with prominent angiogenesis. Downregulation of SCF inhibits tumor-mediated angiogenesis and glioma growth in vivo, whereas overexpression of SCF is associated with shorter survival in patients with malignant gliomas. Thus, the SCF/c-Kit pathway plays an important role in tumor- and normal host cell-induced angiogenesis within the brain.

Tumor angiogenesis is a hallmark of malignant gliomas and is thought to play an important role in glioma progression. SCF expression in glioma cells has been reported, but its significance remains unclear. We have now demonstrated that SCF expression, both by neurons as well as by infiltrating glioma cells, results in a marked angiogenic response in vivo. Downregulation of SCF expression in vivo results in improved survival in orthotopic mouse glioma models, whereas overexpression of SCF is associated with a worse prognosis in patients with glioblastoma. This work, therefore, shows that SCF, expressed by both tumor and normal host cells, is an important antiangiogenic target in malignant gliomas and other SCF-expressing tumors.

Aberrant Expression of SCF in Gliomas

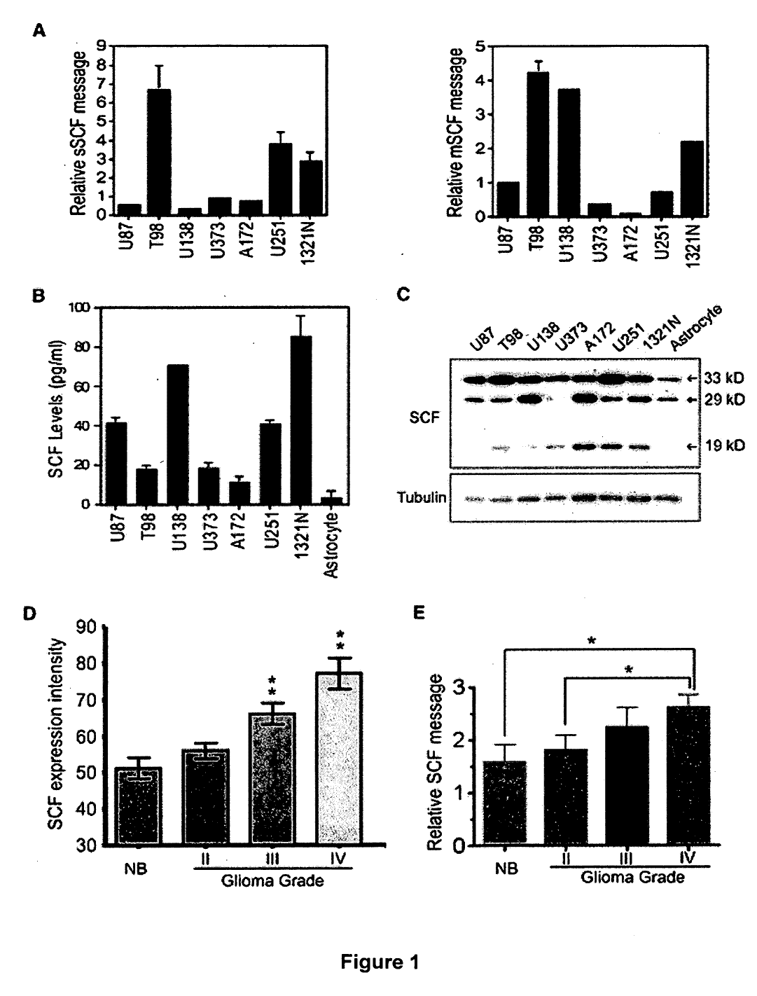

Under normal conditions, little or no SCF expression is detectable in the normal brain (Zhang and Fedoroff 1999 Acta Neuropathol (Berl) 97:393-398 and Manova et al. 1992 J Neurosci 12:4663-4676). To evaluate whether SCF is associated with the prominent angiogenesis seen in malignant gliomas, we first determined SCF expression levels in human malignant glioma cell lines. Quantitative real-time RT-PCR revealed high levels of both the secreted SCF (sSCF) and membrane bound SCF (mSCF) mRNA splicing isoforms in all glioma cells examined (FIG. 1A). We further demonstrate by ELISA that high levels of SCF protein are secreted into the conditioned media (CM) by glioma cell lines in contrast to very low levels found in the CM from normal human astrocytes in vitro (FIG. 1B). Additionally, lysates of glioma cell lines reveal high levels of SCF protein as detected by immunoblotting (FIG. 1C). The three bands seen on the Western blot correspond to the sSCF (33 kDa), cleaved fraction of sSCF (19 kDa), and mSCF (29 kDa).

In order to evaluate the level of SCF expression in primary human gliomas, rather than in just cell lines, we performed mRNA expression profiling of 157 primary human gliomas and 23 nontumor human brain samples using the Affymetrix Human Genome U-133 plus 2.0 GeneChip. A total of four probe sets corresponding to the SCF gene are present, and FIG. 1D demonstrates the data from a typical probe set showing a significant elevation of SCF message in gliomas in comparison to nontumor brain (p<0.001). High-grade gliomas differ significantly from low-grade gliomas both biologically and genetically. Thus, we investigated the levels of SCF expression in gliomas of different grades. The results revealed a statistically significant higher level of SCF expression in high-grade gliomas (grade III or anaplastic astrocytoma, n=31; grade IV astrocytoma or glioblastoma, n=81) compared to low-grade gliomas (grade II astrocytoma, n=45) or normal brain (n=23) (p<0.001) (FIG. 1D). The microarray expression data were confirmed by quantitative real-time RT-PCR demonstrating a significant elevation of SCF in grade IV gliomas compared to low-grade gliomas or nontumor brain (p<0.05) (FIG. 1E). Since high-grade gliomas are characterized by a much greater amount of tumor-associated angiogenesis compared to low-grade gliomas, the positive correlation of SCF expression with increasing glioma grade is consistent with a potential role for SCF in glioma-associated angiogenesis.

Effects of SCF on Brain Microvascular EC

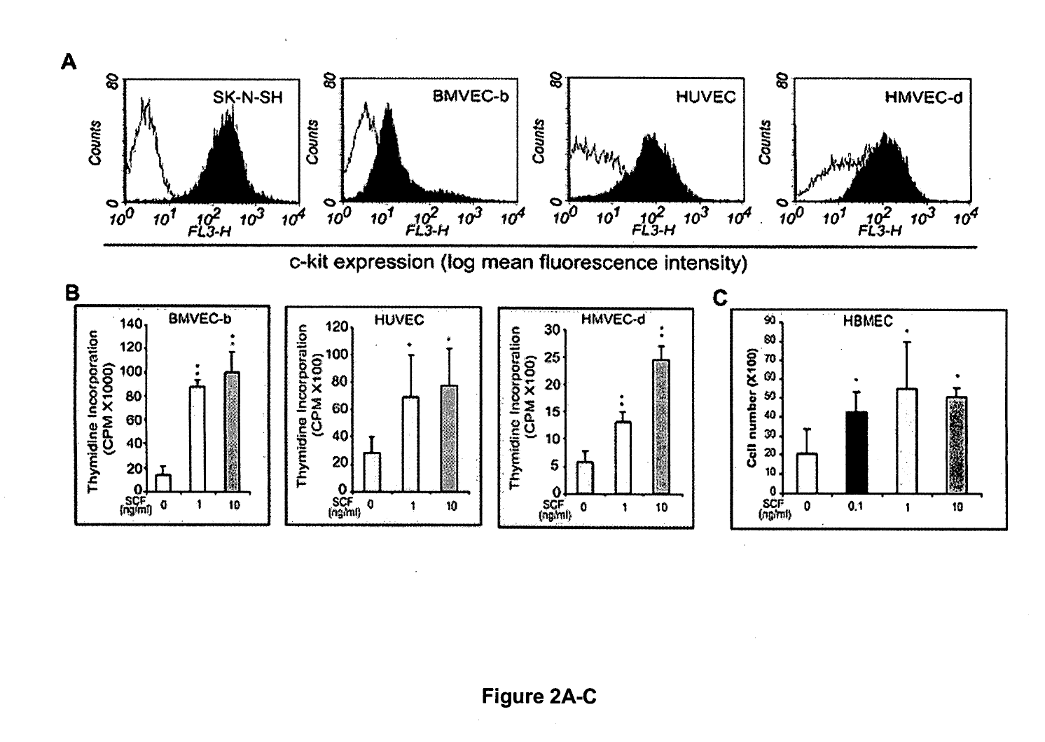

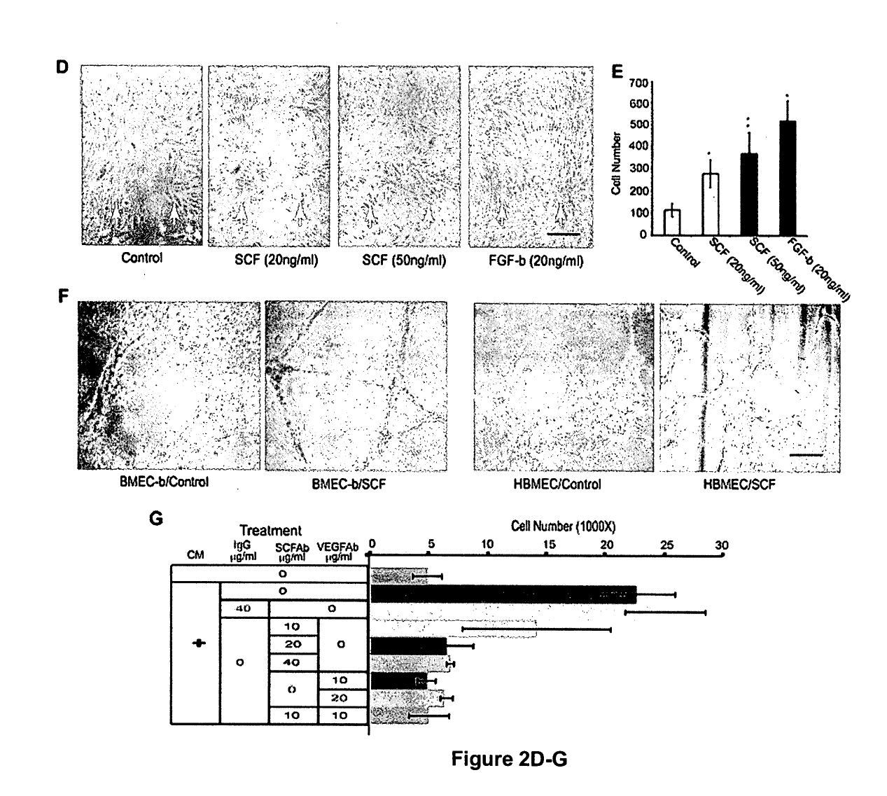

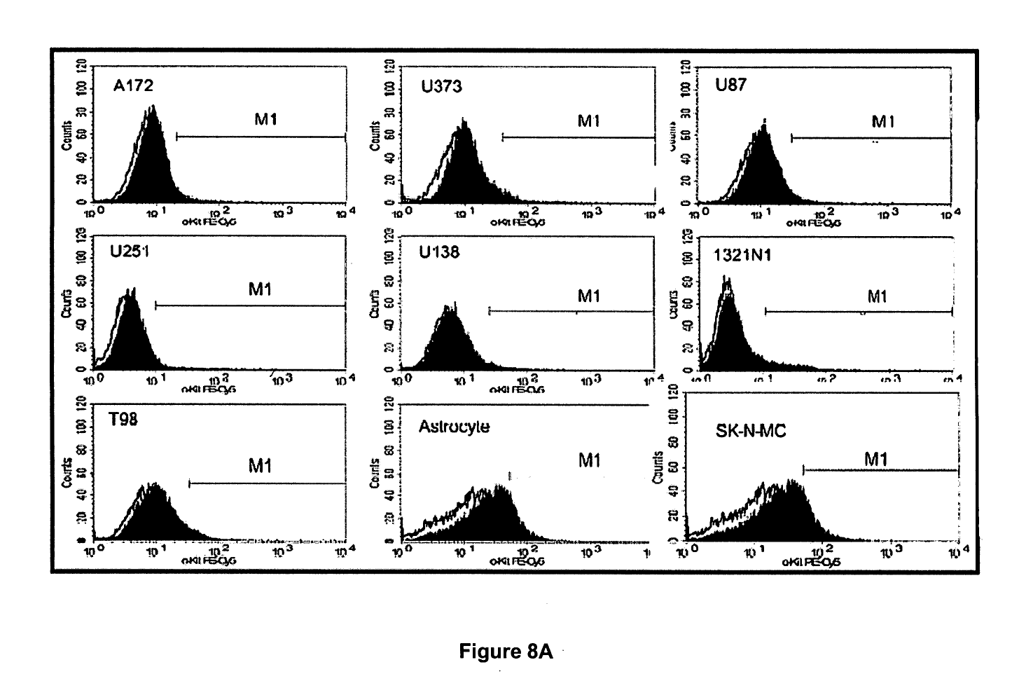

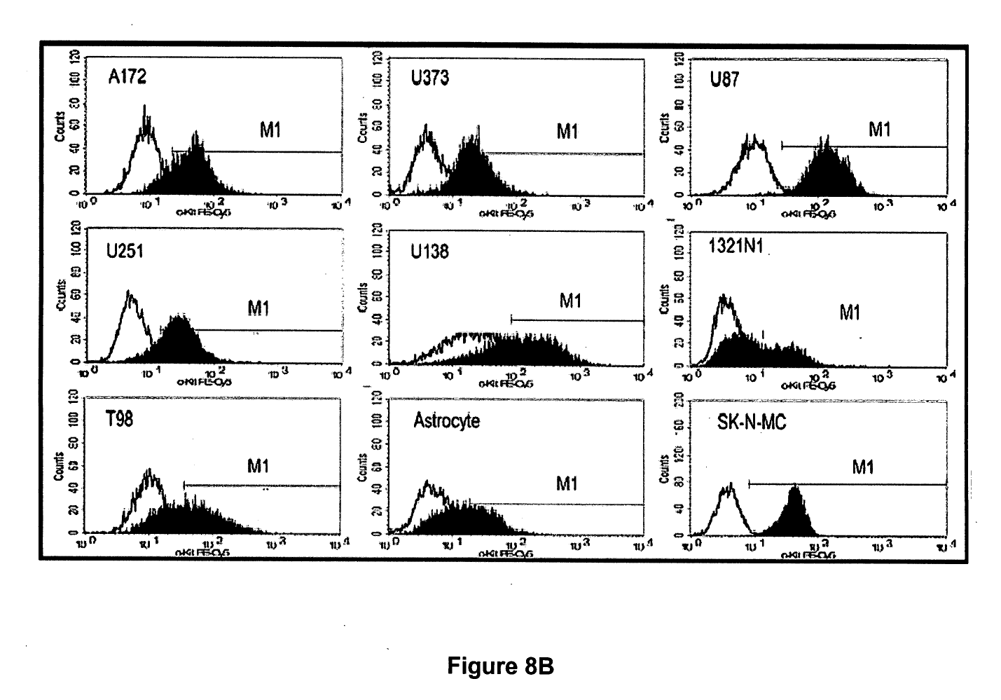

Even though the SCF receptor c-Kit is often expressed on glioma cells in vitro (Natali et al. 1992 Cancer Res 52:6139-6143; Tada et al. 1994 J Neurosurg 80:1063-1073 and Stanulla et al. 1995 Acta Neuropathol (Berl) 89:158-165), even high concentrations of SCF failed to induce proliferation in our glioma cells in vitro, consistent with previous observations (Berdel et al. 1992 Cancer Res 52:3498-3502 and Stanulla et al. 1995 Acta Neuropathol (Berl) 89:158-165). Given the lack of effect of SCF on glioma cell growth in vitro, yet the positive correlation between glioma grade and higher levels of SCF expression, we reasoned that SCF might be involved in glioma progression in vivo through some indirect or paracrine mechanism. To explore the possibility that this indirect effect was a proangiogenic one, we first set out to determine whether c-Kit was expressed on primary ECs in vitro. All ECs examined exhibited c-Kit protein on their cell surfaces by FACS analysis, including bovine brain microvascular EC (BMVEC-b), HUVEC, and human dermal microvascular EC (HMVEC-d) (FIG. 2A). By contrast, all of seven glioma cell lines demonstrated the presence of intracytoplasmic but little membrane bound c-Kit (FIGS. 8A and 8B; Table 1).

TABLE 1
Expression of c-Kit in the glioma cell lines

| | c-Kit membrane expression | | Total c-Kit expression | |
|---|---|---|---|---|
| | | Fold increase in | | Fold increase in |
| | % of cells | fluorescence | % of cells | fluorescence |
| | in M1* | intensity | in M1* | intensity |
| A172 | 4.95 | 1.39 | 81.69 | 5.24 |
| U373 | 5.78 | 1.71 | 59.79 | 4.56 |
| U87 | 5.78 | 1.25 | 99.04 | 14.82 |
| U251 | 4.60 | 1.69 | 93.19 | 5.30 |
| U138 | 3.45 | 1.19 | 43.86 | 4.04 |
| 1321N1 | 10.37 | 2.09 | 38.32 | 9.14 |
| T98 | 6.81 | 1.85 | 53.33 | 5.28 |
| Astrocyte | 18.06 | 2.02 | 61.64 | 7.45 |
| SK-N-MC | 98.71 | 19.44 | 99.85 | 9.39 |

*M1 markers have been set at the 98% confidence limit and are shown in histograms (FIG. 8)

We next exposed BMVEC-b, HUVEC, and HMVEC-d in basal medium to SCF and determined the rate of thymidine incorporation at 48 hr or 72 hr. SCF stimulated proliferation in all three primary ECs in a dose-dependent manner even at low concentrations (i.e., BMVEC-b proliferation increased approximately 6-fold and 7-fold following exposure to 1 ng or 10 ng of SCF per ml for 72 hr, respectively) (FIG. 2B). Additionally, we exposed human brain microvascular EC (HBMEC) to various concentrations of SCF (from 0.1 to 10 ng per ml) and demonstrated a similar dose-dependent pro-proliferative response to that seen with the BMVEC-b (FIG. 2C). It is particularly noteworthy that SCF alone, without the addition of other cytokines such as VEGF, was sufficient to sustain human brain microvascular EC and other EC cell growth and survival.

We next used a wound healing assay and Matrigel tube formation assay to further characterize the effects of SCF on BMVEC-b migration and differentiation in vitro. SCF induced a 2- to 3-fold increase in BMVEC-b migration compared to control in a dose-dependent manner (FIGS. 2D and 2E). Additionally, SCF induced BMVEC-b and HBMEC to migrate in Matrigel within 4 hr of exposure and to form mature appearing capillary-like structures by 18 hr. By contrast, these primary human and bovine brain ECs exposed to the vehicle control neither migrated nor formed capillary-like structures (FIG. 2F). In order to establish whether glioma cells could be exerting a proangiogenic effect on neighboring ECs through SCF and other angiogenic proteins, we evaluated the effects of glioma cell CM on EC proliferation. EC proliferation increased 5-fold following exposure to glioma CM compared to basal media. Blocking antibodies to both SCF and VEGF were able to abolish the mitogenic effects of the glioma CM on EC proliferation (FIG. 2G). These data demonstrate the ability of SCF to induce proliferation, migration, and differentiation of primary brain microvascular ECs in vitro.

SCF/c-Kit Activates Signal Transduction in EC

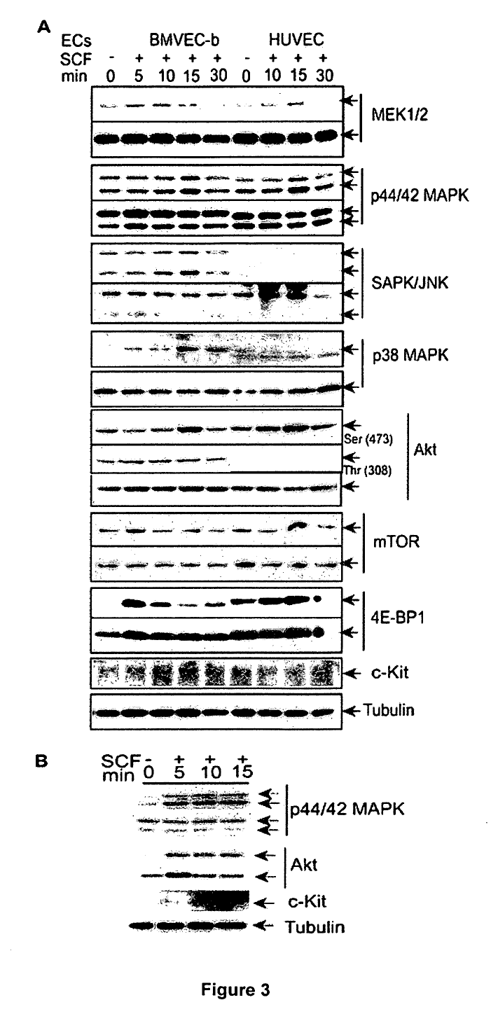

SCF-mediated c-Kit signaling has been shown to induce activation of the mitogen-activated protein kinase (MAPKs) and phosphoinositide 3-kinase (PI3K)/Akt pathways in known SCF target cells (Jin, 2005 Mol Reprod Dev 70:82-90). A recent report suggests that SCF stimulates a HUVEC angiogenic response through activation of these pathways (Matsui et al. 2004 J Biol Chem 279:18600-18607). We investigated whether SCF could induce these two pathways specifically in primary brain ECs rather than just in HUVEC. Confluent BMVEC-b and HUVEC cells were treated with SCF after serum starvation. SCF rapidly induced c-Kit auto-tyrosine-phosphorylation, which peaked at 10-15 min and lasted for 30 min following exposure (FIG. 3A). Additionally, phosphorylation of MEK1/2, as well as its downstream p44/42 MAPK, was dramatically increased. SAPK/JNK was similarly activated 10-15 min following SCF exposure, whereas p38 MAPK exhibited a prolonged activation for over 30 min.

Like the MAPK pathway, the PI3K pathway was activated following BMVEC-b and HUVEC exposure to SCF. Phosphorylation of Akt was significantly enhanced by SCF treatment, which in turn resulted in phosphorylation of mTOR. Activation of mTOR increased phosphorylation of 4E-BP1 consistent with a possible diminished inhibition of 4E-BP1 on eIF4E, which could result in diminished cap-dependent translation. Thus, SCF activates the MAPK and PI3K pathways in BMVEC-b in a manner similar to its effects on HUVEC.

Additionally, SCF (50 ng/ml) induced immediate phosphorylation of the c-Kit, p44/42 MAPK, and Akt proteins in HBMECs (FIG. 3B). Thus, SCF-induced activation of the MAPK and P13K pathways appears to occur in ECs from both systemic capillary beds as well as from the brain. In contrast to BMVEC-b, SCF could not induce phosphorylation of p38 MAPK and SAPK/JNK in HBMEC. Whether this represents a species-specific difference in SCF-mediated signaling or reflects different subtypes of ECs from the brain remains to be determined.

SCF Promotes Angiogenesis in vivo

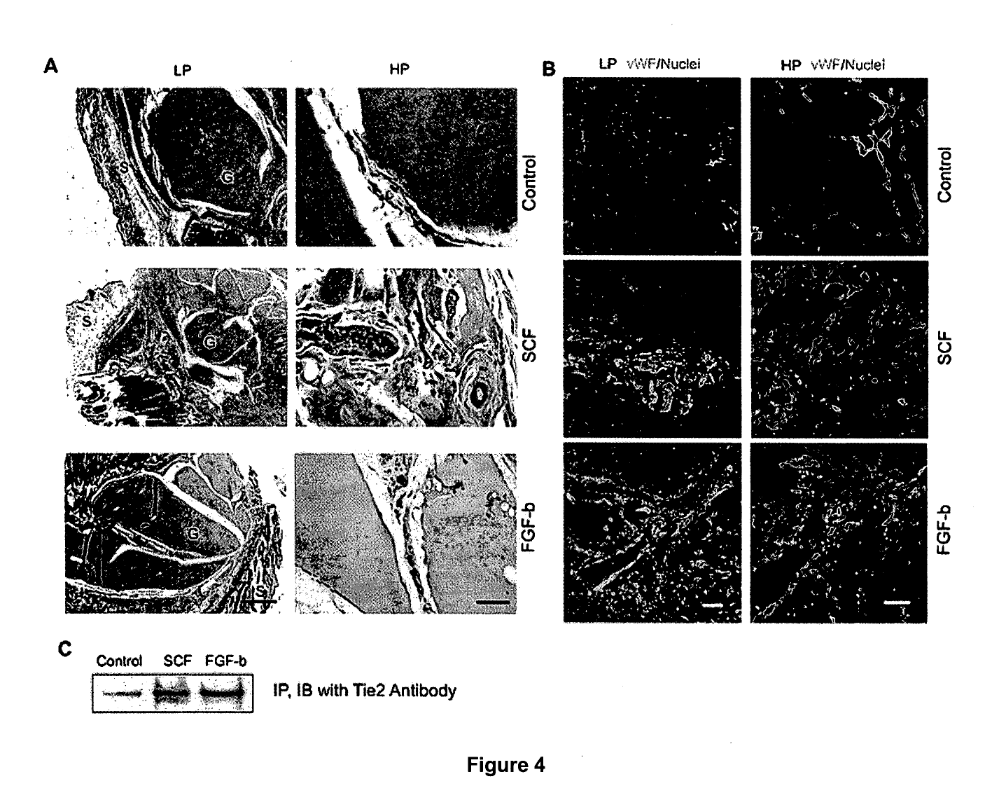

We next evaluated whether SCF has proangiogenic activity in vivo by subcutaneously implanting Matrigel impregnated with SCF, β-FGF (positive control), or vehicle alone into the adult SCID mice. Newly formed blood vessels in the Matrigel plugs were visualized by hematoxylin/eosin (H&E) staining and confirmed by immunofluorescent staining for factor VIII-related antigen (vWF)-positive ECs. In addition, Tie2 protein levels in the Matrigel plugs were determined. Fourteen days after injection, vehicle control-impregnated Matrigel plugs demonstrated few vWF-positive vessels and had essentially no detectable Tie2 protein expression (FIG. 4). By contrast, SCF (as well as the positive control β-FGF) induced substantial angiogenesis, as demonstrated by abundant intact blood vessels as well as high Tie2 expression within the SCF-containing Matrigel plug. These data demonstrate that SCF can promote angiogenesis in vivo.

Silencing SCF Attenuates Glioma Angiogenesis

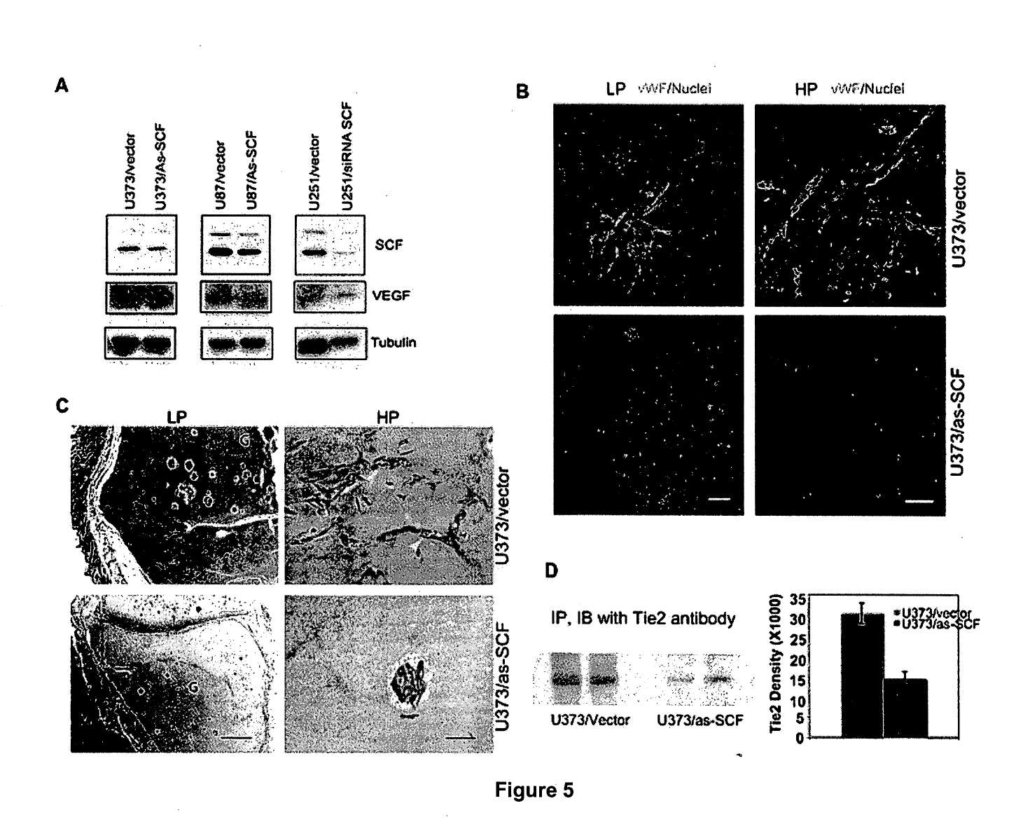

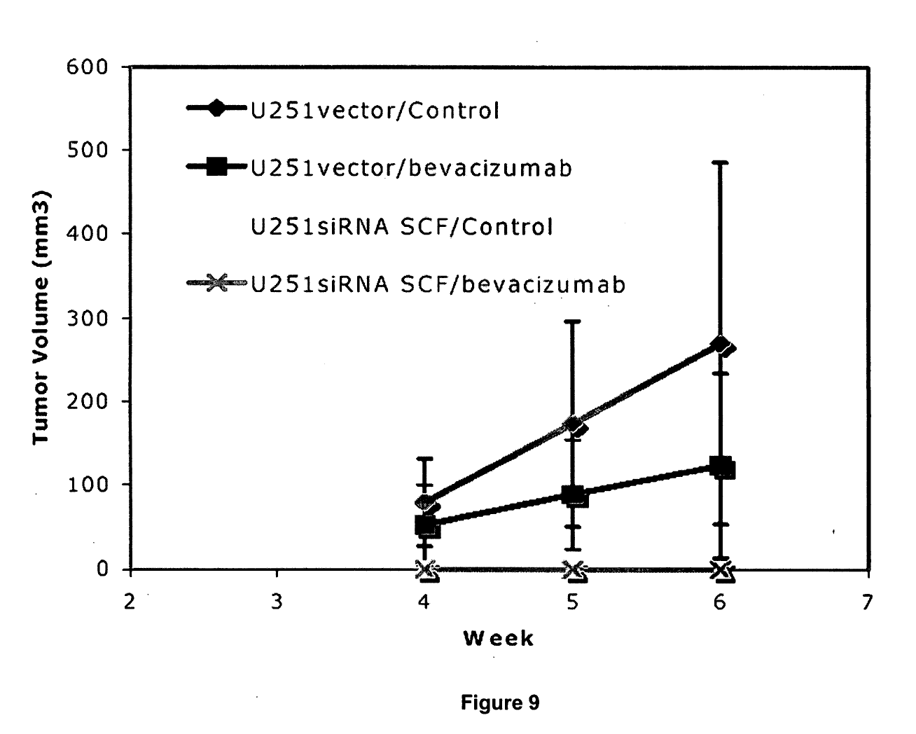

To evaluate the effect of SCF on glioma angiogenesis and progression, we engineered glioma cells expressing low levels of SCF by retrovirally transducing an antisense SCF construct or a SCF shRNA into wild-type U373, U87, and U251 glioma cells expressing high levels of SCF. The resulting U373 cell line (U373/as-SCF), U87 cell line (U87/as-SCF), and U251 cell line (U251/shRNA SCF) were expanded, and then stable low level-expressing clones were selected by antibiotics and verified by Western blot (FIG. 5A). To avoid the confounding variable of concurrent VEGF expression, we verified by Western blots that suppression of SCF expression in our cloned glioma cell lines had no effects on VEGF expression (FIG. 5A). Next, we implanted U373/as-SCF or U373/vector cell lines together with Matrigel subcutaneously into SCID mice in order to assess glioma-induced angiogenesis in vivo with and without SCF stimulation. Within 2 weeks, a profound angiogenic response was seen in the Matrigel containing the control U373/vector cells with multiple vWF-positive perfused vessels and abundant Tie2 protein in the Matrigel plug homogenates. By contrast, Matrigel containing the U373/as-SCF cells demonstrated far fewer capillaries with vWF staining and significantly less Tie2 protein in the plug homogenates (FIGS. 5B-5D). Thus, suppression of SCF in glioma cells results in significant inhibition of glioma-induced angiogenesis in vivo. Consistent with these observations, U251/as-SCF did not form palpable subcutaneous tumors several weeks after implantation, in contrast to their matched U251/vector cells, which formed tumors so large that animals needed to be euthanized (FIG. 9). Likewise, systemically administered bevacizumab (Genentech, San Francisco, Calif.) inhibited U251/vector produced VEGF resulting in profound tumor growth suppression. We could not, however, demonstrate an additive or synergistic effect of SCF knockdown and VEGF inhibition, since SCF knockdown alone resulted in complete tumor growth inhibition (FIG. 9).

SCF Effects on Survival in Intracranial Tumor Model

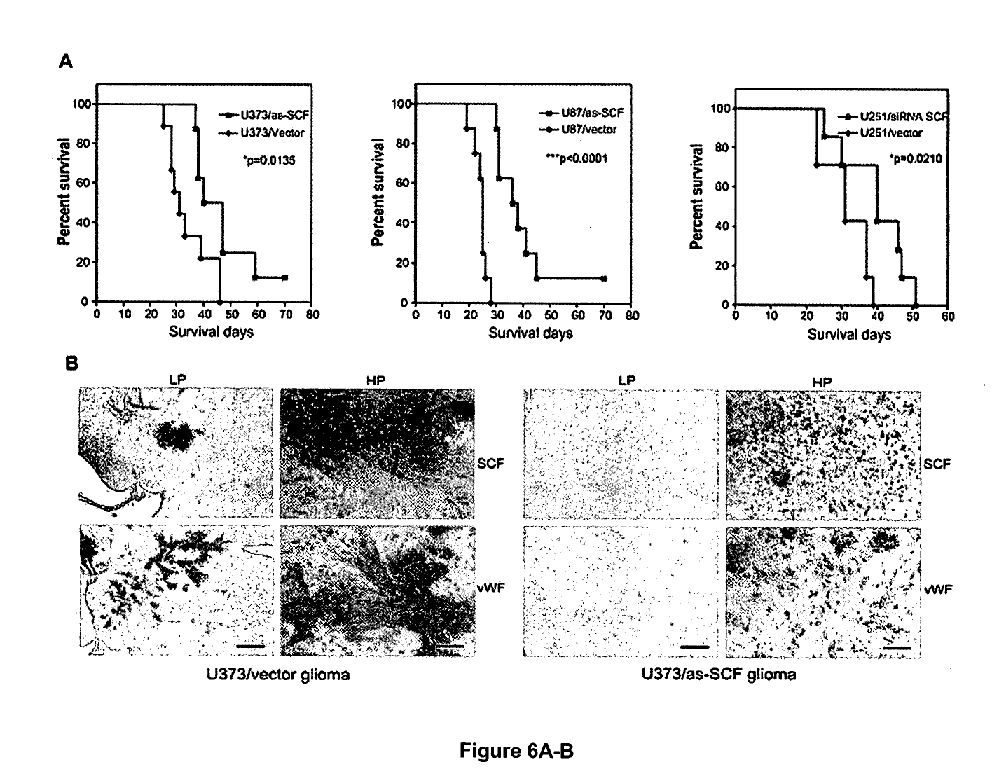

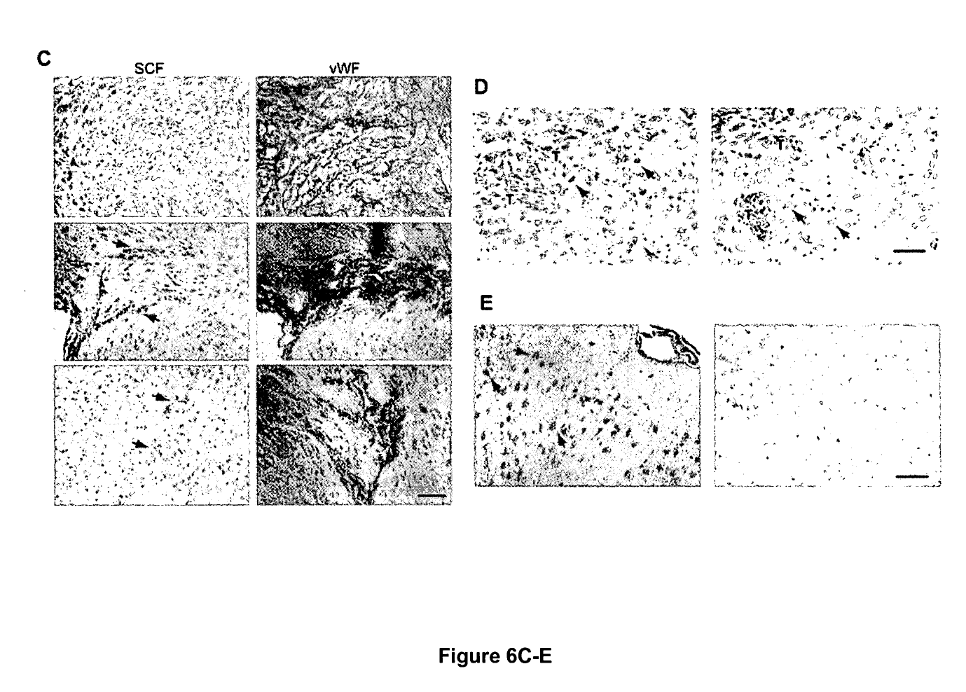

We next evaluated whether suppression of SCF would affect the survival of animals with intracranial gliomas. U373/as-SCF or U373/vector cells, U87/as-SCF or U87/vector cells, and U251/shRNA SCF or U251/vector cells were stereotactically implanted into the cerebral subcortex of adult athymic nu/nu mice. Log-rank analysis of the Kaplan-Meier survival curves demonstrated a significant survival advantage for the SCF low-expression glioma-bearing mice compared to their matched SCF high-expression parental vector-infected glioma-bearing animals (p<0.05) (FIG. 6A). Examination of 4-week-old U373/vector intracranial tumors demonstrated significant SCF expression within the tumor cells and a robust angiogenic response. By contrast, there was significantly lower SCF expression within the tumor cells of the intracranial U373/as-SCF tumors with an attenuated angiogenic response within the tumor (FIG. 6B).

Immunohistological staining demonstrated that, although SCF is generally expressed in most tumor cells within the central U373/vector tumor mass, SCF expression was most marked in tumor cells within the tumor border and in the few infiltrating glioma cells at the invasion front. Increased SCF expression was also observed in the normal brain tissue adjacent to the tumor mass (FIG. 6C). In situ hybridization for SCF mRNA revealed that it was the normal host neurons within the cerebral cortex, adjacent to the invading tumor mass, that accounted for the non-tumor cell-associated high-level SCF expression (FIG. 6D). Additionally, a similar pattern and level of normal neuronal overexpression of SCF was seen in cortex immediately adjacent to U373/as-SCF tumors despite the fact that the actual tumor cells made very little SCF (in contrast to the U373/vector cells). Normal neurons at increasingly farther distances from the tumor site, however, demonstrated progressively less SCF expression, such that SCF expression was barely detectable in neurons within the cortex contralateral to the growing tumor mass (FIG. 6E). Together, these results support a role for tumor cell and normal host neuron-mediated SCF-induced tumor angiogenesis in glioma progression.

Biological and Clinical Significance of SCF in Glioma-Infiltrated Human Cerebral Cortex

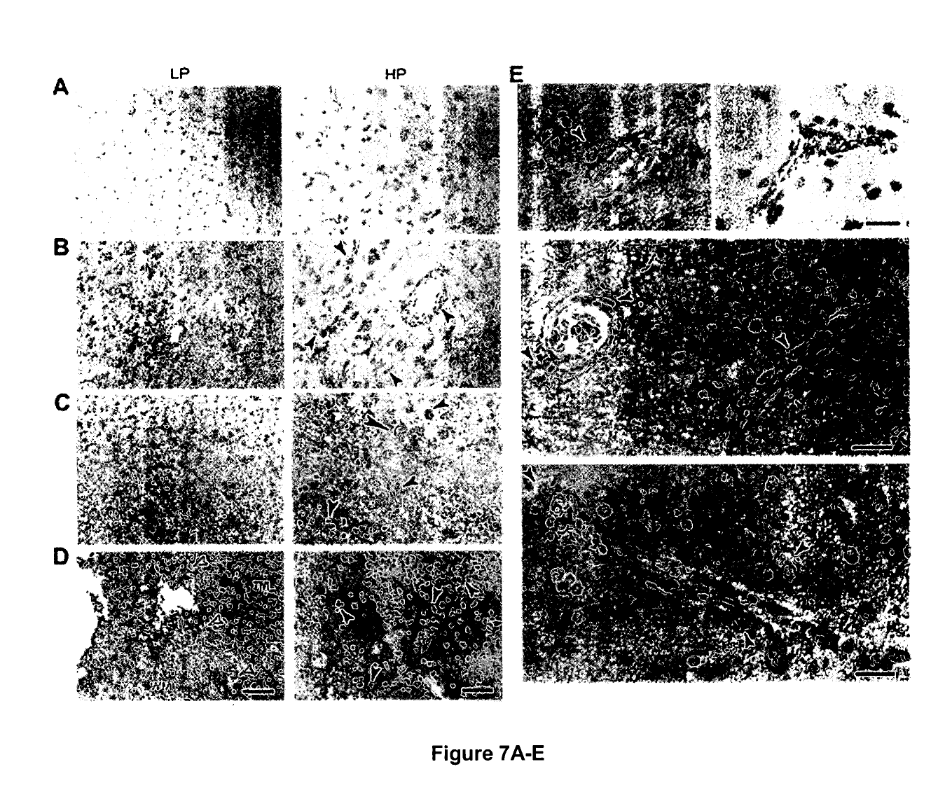

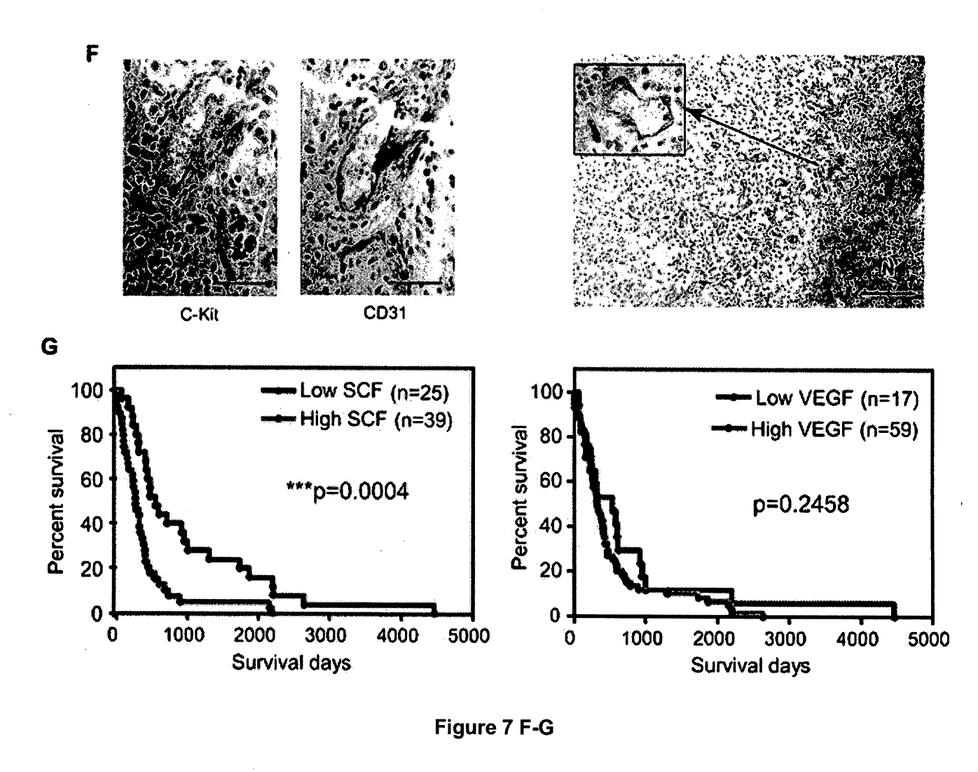

To evaluate whether the patterns of glioma-associated SCF expression in the intracranial mouse xenograft model reflected patterns seen in human brains with malignant gliomas, we performed extensive immunohistochemical analysis of surgical specimens from patients with glioblastoma. We observed generally four important features of the heterogeneous SCF expression in the brains of patients with glioblastoma. In some areas, close to the center of tumor mass, very little or no SCF was detected (FIG. 7A). SCF was generally expressed at high levels by glioma cells infiltrating into the cerebral white matter (FIG. 7B). In the glioma-infiltrated cerebral cortex abundant SCF expression was seen not only in the tumor cells, but also in normal host neurons (FIG. 7C). Thus, there was profound expression of SCF in cerebral cortex infiltrated by glioma cells secondary to both tumor- and normal host neuron-associated SCF expression (FIG. 7D). In summary, SCF expression appears to reside most prominently in the invasive front of the infiltrating glioma, suggesting its roles in tumor progression. Most notably, this area of high SCF expression colocalizes with areas of dense microvasculature sprouting and branching, consistent with a role for SCF in glioma- and normal host neuron-mediated angiogenesis (FIG. 7E). In addition, abundant c-Kit expression was also detected on the glioblastoma-associated ECs but little on the actual tumor cells, consistent with our in vitro data (FIG. 7F). The clinical significance of these findings is reflected by the fact that patients with glioblastoma (grade IV; n=64), otherwise matched for known prognostic factors (i.e., age, treatment), who had tumors that expressed high levels of SCF experienced significantly shorter survival than patients with tumors expressing relatively low levels of SCF (p=0.0004 by log-rank analysis; FIG. 7G). By contrast, VEGF expression levels did not correlate with survival in patients with glioblastoma (p=0.24).

### DISCUSSION

The work reported here describes a role for SCF in tumor-associated angiogenesis and specifically in glioma progression. SCF has been long recognized as an important growth factor for a number of cell types, including hematopoietic stem cell, mast cells, melanocytes, and germ cells (Zsebo et al. 1990 Cell 63:213-224; Mackenzie, M. A. et al. 1997 Dev Biol 192:99-107). In these divergent cell types, SCF has been shown to have a number of different biological properties. A role for SCF as a potential angiogenic factor, however, has not been previously entertained until a recent report demonstrated its mitogenic effects on HUVEC cells in vitro (Matsui et al. 2004 J Biol Chem 279:18600-18607). We have now expanded on those observations by demonstrating that SCF has potent mitogen, differentiation, and chemotactic properties on a variety of primary EC lines in vitro, and furthermore can induce a robust angiogenic response and contribute to tumor-associated angiogenesis in vivo.

It has been previously reported that glioma cell lines can express SCF and c-Kit, implicating SCF as an autocrine growth factor for gliomas (Hamel and Westphal, 2000 Acta Neurochir (Wien) 142:113-137). Previous observations, however, could not demonstrate a mitogenic effect of SCF on glioma cells. Thus, the role for SCF in glioma pathogenesis remained unclear until now. Our data now confirm that SCF expression is not a direct glioma mitogen but rather a potent glioma-associated angiogenic factor. Although SCF has been previously implicated in angiogenesis through its ability to regulate mast cell migration with subsequent release of VEGF (Zhang et al. 2000 Cancer Res 60:6757-6762), no direct role for SCF in angiogenesis had been previously described except for a recent report of its ability to induce HUVEC proliferation and migration in vitro (Matsui et al. 2004 J Biol Chem 279:18600-18607). We have extended those observations by showing that not only HUVECs, but also primary ECs (including human brain microvascular ECs) express c-Kit and that SCF-mediated c-Kit signaling in ECs results in activation of the MAPK and AKT pathway, leading to enhanced proliferation, survival, and migration of ECs even in the absence of VEGF, β-FGF, or other, thought to be obligate, EC growth factors.

Our data clearly demonstrate the ability of SCF to induce a potent angiogenic response both subdermally and within the brain. Furthermore, we show using several different cell lines and using several different methodologies that downregulation of SCF expression in an orthotopic glioma model results in diminished angiogenesis and prolongation of animal survival. We believe that these observations have clinical significance based on our demonstration that the level of SCF expression in primary human gliomas directly correlates with the grade of the tumor and patient survival. These data demonstrating that high-grade gliomas express significantly more SCF than low-grade gliomas or nontumor human brain tissue are fully consistent with the fact that high-grade gliomas are significantly more angiogenic than low-grade gliomas. In fact, endothelial proliferation is one of the pathological criteria for grading a glioma high grade (Kleihues and Cavenee, 2000 Pathology and Genetics of Tumors of the Nervous System, International Agency for Research on Cancer, Lyon). Additionally, our demonstration of increased angiogenic activity colocalizing with increased SCF expression in surgical sections of human brains with malignant gliomas further supports the relevance of SCF-induced angiogenesis in the pathogenesis of malignant gliomas.

It is becoming increasingly clear that tumorigenesis in vivo involves not just the biology of the tumor cell itself, but rather a complex interplay between tumor cells and the deregulation of normal host physiological functions that ultimately aid in tumor progression (Tlsty and Hein, 2001 Curr Opin Genet Dev 11:54-59 and Liotta and Kohn, 2001 Nature 411:375-379). Others and we have previously demonstrated that SCF expression is induced in normal neurons in vivo following various types of brain trauma (Zhang and Fedoroff 1999 Acta Neuropathol (Berl) 97:393-398 and Sun et al. 2004 J Clin Invest 113:1364-1374). Gliomas cause significant damage to normal brain parenchyma through numerous mechanisms including disruption of the blood-brain barrier with resultant increased cerebral edema and increased intracranial pressure, glioma-mediated degradation of the extracellular matrix through overexpression of metalloproteases, and release of neurotoxic excitatory molecules such as glutamate (Forsyth et al. 1999 Br J Cancer 79:1828-1835; Takano et al. 2001 Nat Med 7:1010-1015; VanMeter et al. 2001 J Neurooncol 53:213-235; Davies, 2002 J Anat 200:639-646 and Chantrain et al. 2004 Cancer Res 64:1675-1686). Thus, we wondered whether, along with the direct release of SCF from glioma cells, tumor growth could be eliciting expression of SCF from normal brain tissue in a manner analogous to that seen in our brain injury models. Data from both our orthotopic model and the surgical sections from human brains with malignant gliomas demonstrate that normal host neurons within the area of cerebral cortex, adjacent to the tumor mass and within the invading front of glioma cells, do express SCF to very high levels, whereas neurons in areas of cortex away from the growing tumor do not. These areas of neuronal and glioma SCF overexpression overlap precisely with areas of sprouting and branching angiogenesis. Thus, normal host cells within the brain appear to have been coopted to induce pathological angiogenesis in support of the infiltrating tumor cells. The expression of SCF by normal host tissue may also help explain why downregulation of direct glioma SCF expression through our antisense and siRNA constructs in our orthotopic models decreased glioma-mediated angiogenesis but did not abolish it and only led to moderately prolonged animal survival. Full angiogenesis inhibition will likely depend on inhibition of all SCF expression (or c-Kit inhibition), including both tumor- and normal host neuron-mediated expression, possibly in conjunction with inhibition of other angiogenic pathways.

These data strongly implicate SCF as an important angiogenic factor in pathogenesis of malignant gliomas. Among the relatively large number of factors that have been implicated in glioma-mediated angiogenesis, the most commonly cited are β-FGF, PDGF, and VEGF (Dunn et al. 2000 J Neurooncol 50:121-137 and Lamszus et al. 2004 Cancer Treat Res 117:169-190). Although gliomas clearly express β-FGF in a grade-dependent manner, various studies have failed to find β-FGF receptors on glioma-associated endothelium, leading some to speculate that β-FGF is acting more as a glioma autocrine growth factor rather than an angiogenic factor (Zagzag et al. 1990 Cancer Res 50:7393-7398 and Morrison et al. 1994 J Neurooncol 18:207-216). The various heterodimer PDGF receptors have been demonstrated on glioma-associated endothelium; however, PDGF overexpression tends to occur in low-grade gliomas and higher-grade gliomas that have transformed from low-grade gliomas rather than in the more common primary glioblastomas (Westermark et al. 1995 Glia 15:257-263; Ribom et al., 2002 and Dai et al. 2001 Genes Dev 15:1913-1925). Thus, PDGF may also be playing a largely mitogenic autocrine role in glioma growth and may not have a major angiogenic function in the majority of high-grade gliomas. VEGF is the one angiogenic factor that has clearly been implicated in glioma angiogenesis, as it is highly overexpressed in most high-grade gliomas and especially in glioblastoma, where its receptor is overexpressed on glioma-associated endothelium (Berkman et al. 1993 J Clin Invest 91:153-159 and Hatva et al. 1995 Am J Pathol 146:368-378). VEGF expression, however, has been demonstrated to be most pronounced in glioblastoma cells adjacent to areas of necrosis, consistent with its known hypoxia inducibility (Plate et al. 1992 Nature 359:845-848 and Shweiki et al. 1992 Nature 359:843-845). These areas of necrosis and relative tumor hypoxia, however, are not the areas of the most pronounced angiogenesis or most rapid tumor cell proliferation within a malignant glioma. Rather, it is the invading border of the tumor that harbors the most prominent angiogenic reaction and tumor cell proliferation (VanMeter et al. 2001 J Neurooncol 53:213-235). This is the area that corresponds to the highest SCF expression.

Our interpretation of these data leads us to believe that SCF, along with VEGF, may have complementary roles in the robust angiogenic response seen in malignant gliomas. To date, there have been a number of clinical trials with small molecule inhibitors of FLK-1 (the VEGF receptor); however, none have yet to show definitive clinical benefit. Although the lack of clinical success may be secondary to the failings of the individual molecules tested, it is also plausible to postulate that angiogenic pathways other than or in addition to VEGF will need to be targeted for clinical success. As such, SCF may prove to be an important therapeutic target in high-grade gliomas. There has already been one clinical trial of Imatinib (Gleevec), a small molecular inhibitor of ABL, PDGF, and c-Kit, in patients with malignant gliomas primarily based on the premise of targeting PDGF as an autocrine growth factor (Kilic et al. 2000 Cancer Res 60:5143-5150; P. Y. Wen et al., 2002, Proc. Am. Soc. Clin. Oncol., abstract). The investigators in this trial did not observe any clinical activity from Imatinib; however, the lack of activity may have been the result of the inability of Imatinib to cross the BBB. Although it has been argued that one of the theoretical appeals of antivascular and antiangiogenic therapy for brain tumors is the non-necessity for molecules to cross the BBB, the reality is that many of the endothelial targets may not be accessible to BBB-impermeable molecules within the bloodstream if such targets are present on the adluminal side of the EC. Indeed, given that the BBB works in both directions, a large protein such as SCF would not easily cross out of the brain back into the bloodstream. Thus, it is possible that c-Kit expression may not be expressed on the side of the endothelial cell facing the blood vessel lumen, thereby making the receptor inaccessible to BBB-impermeable drugs such as Imatinib. It remains to be seen whether some of antitumor effects of Imatinib activity against certain systemic solid tumors, such as gastrointestinal stromal tumors (GIST), and chronic myelogenous leukemia (CML) may be in part a result of its antiangiogenic as well as its direct antitumor effects (Demetri et al. 2002 N Engl J Med 347:472-480; Kantarjian et al. 2002 N Engl J Med 346:645-652 and Sawyers et al. 2002 Blood 299:3530-3539).

In conclusion, we have presented data that demonstrate SCF to be a potent angiogenic factor both in systemic tissue and within the brain. Additionally, SCF appears to be an important angiogenic factor in high-grade gliomas, both through direct tumor cell expression of SCF and through the induction of its expression by normal host neurons within areas of brain infiltrated by tumor. Normal neuronal expression of SCF in response to traumatic brain injury also raises the disturbing possibility that standard invasive procedures such as surgical biopsies or partial tumor resections may be inducing a proangiogenic response, or trigger, within the brain. As such, SCF represents an important therapeutic target for malignant gliomas and other SCF-expressing tumors. Finally, the ability of normal host neurons to express SCF in response to injury, as well as the potent angiogenic properties of SCF within the brain, leads one to conclude that SCF might have important therapeutic potential as a proangiogenic factor in regenerative tissue repair strategies for various disease states such as stroke and brain and spinal cord injury as well as neurodegenerative diseases.

Neuronally Expressed Stem Cell Factor Induces Neural Stem Cell Migration to Areas of Brain Iniury

Neural stem/progenitor cell (NSPC) migration toward sites of damaged central nervous system (CNS) tissue may represent an adaptive response for the purpose of limiting and/or repairing damage. Little is known of the mechanisms responsible for this migratory response. We constructed a cDNA library of injured mouse forebrain using subtractive suppression hybridization (SSH) to identify genes that were selectively upregulated in the injured hemisphere. We demonstrate that stem cell factor (SCF) mRNA and protein are highly induced in neurons within the zone of injured brain. Additionally, the SCF receptor c-Kit is expressed on NSPCs in vitro and in vivo. Finally, we demonstrate that recombinant SCF induces potent NSPCs migration in vitro and in vivo through the activation of c-Kit on NSPCs. These data indicate that the SCF/c-Kit pathway is involved in the migration of NSPCs to sites of brain injury and that SCF or SCF-like polypeptides are envisioned as proving useful for inducing progenitor cell recruitment to specific areas of the CNS for cell-based therapeutic strategies.

SCF Expression Induced by “freeze” Injury in Brain

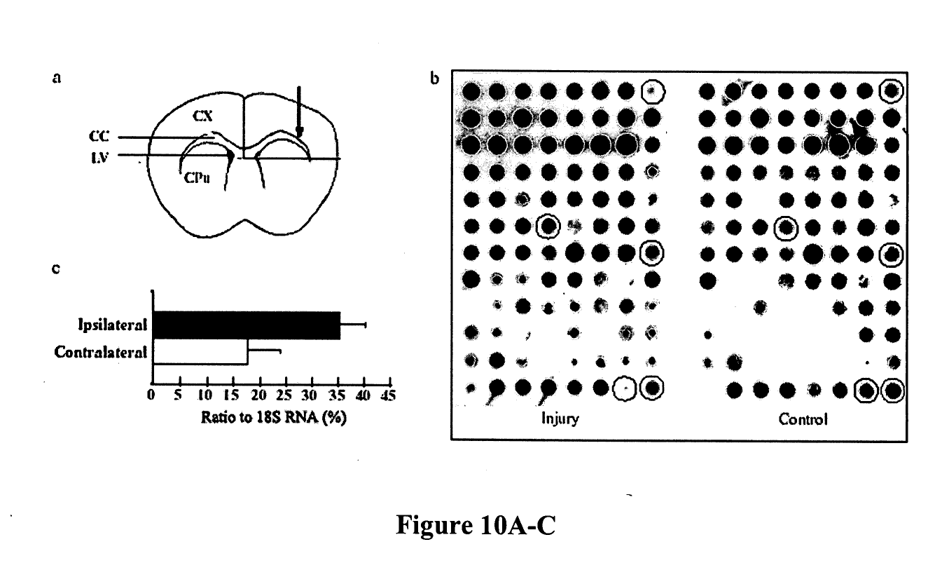

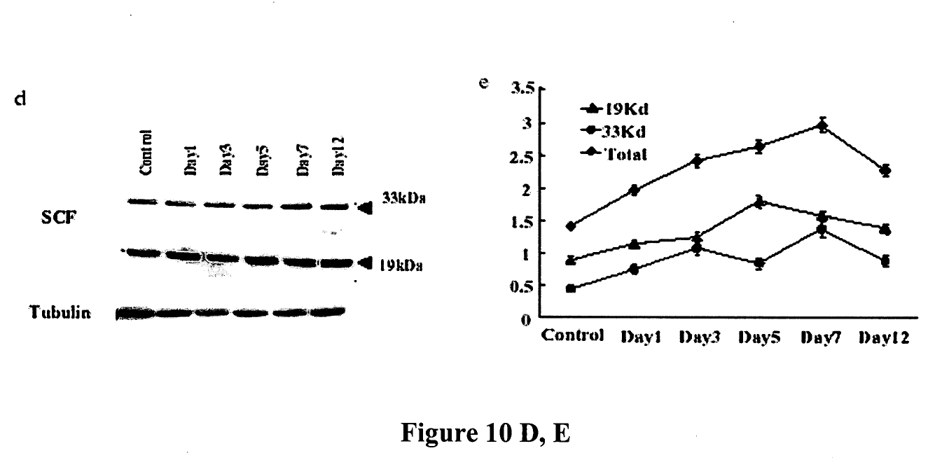

“Freeze” injuries were introduced into the right frontal lobe of mouse brain as illustrated in FIG. 10A. Genes that were differentially expressed in brain tissue 5 days after injury were globally screened through SSH using contralateral uninjured tissue as the control. The differential expression of the isolated clones was verified using custom microarray. More than 300 clones, representing 196 unique genes, were identified. The microarray membranes, which contained the individual clones from the SSH library in duplicate, were hybridized with cDNA from injured frontal lobe or with cDNAs from the uninjured contralateral side. The differentially expressed clones were subsequently sequenced and two of them were identified as SCF (FIG. 10B). The injury-mediated induction of SCF mRNA expression was further confirmed by quantitative real-time RT-PCR, revealing an increase of more than two-fold in SCF transcripts in the injured tissue (FIG. 10C).

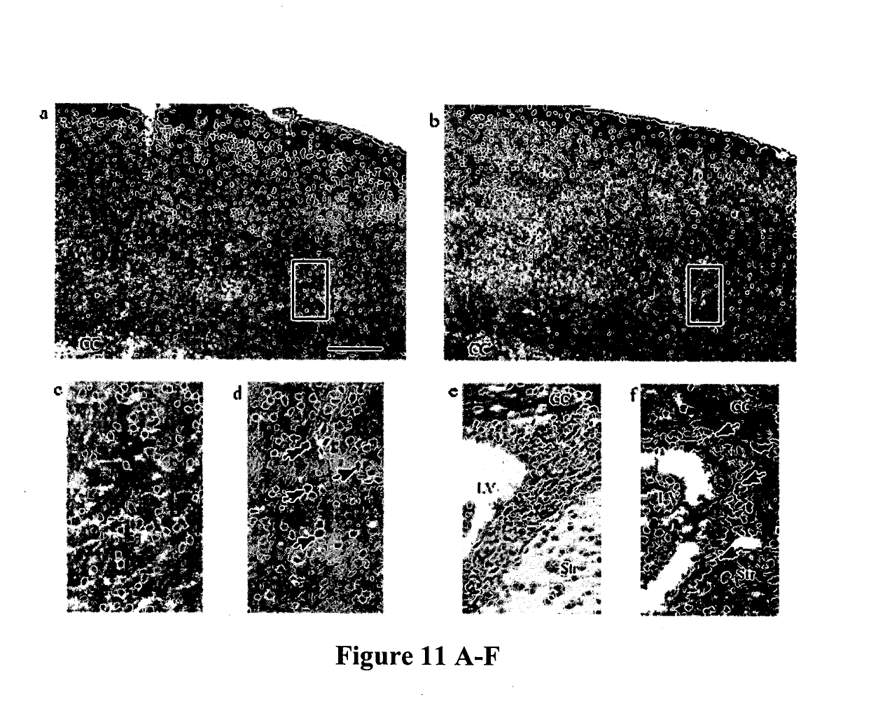

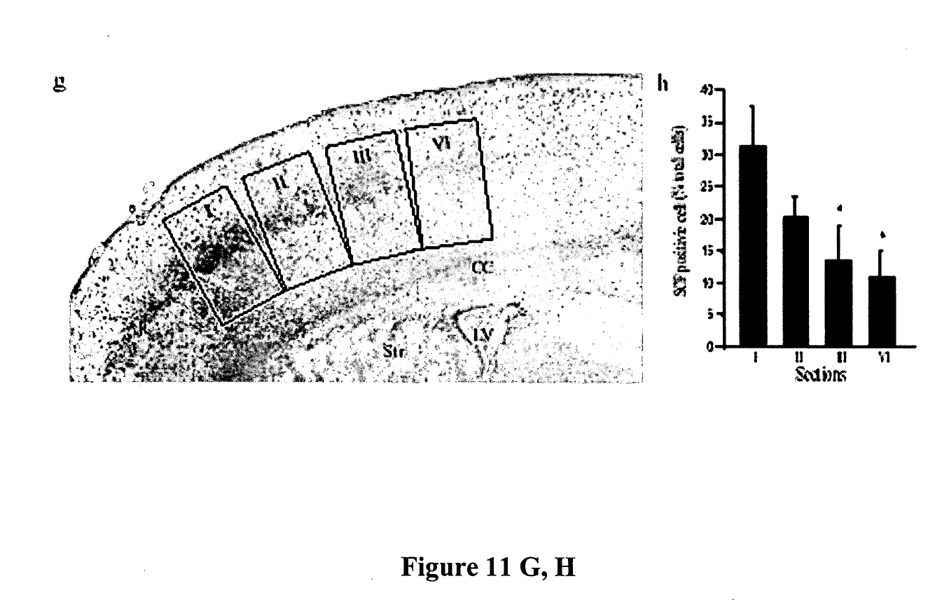

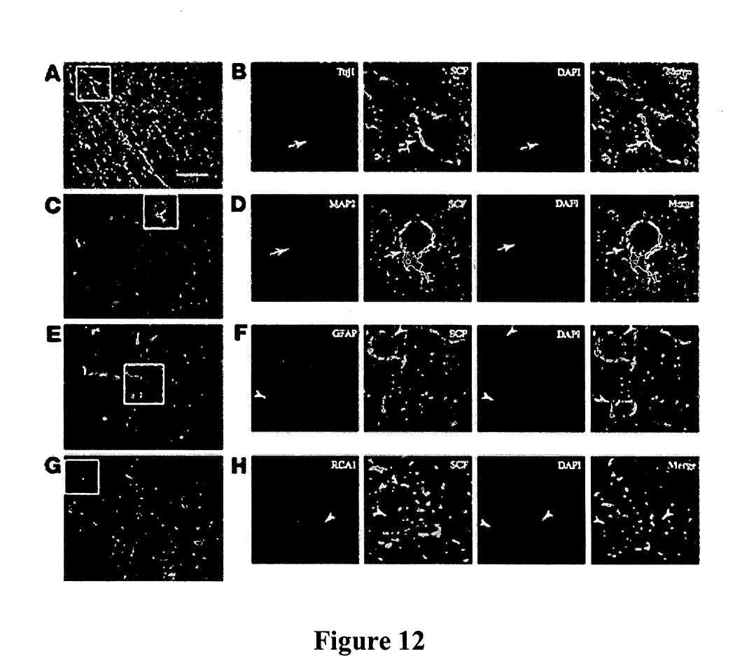

We next investigated whether SCF protein, in parallel to SCF mRNA, was also increased in the injured brain. Western blot confirmed the presence of two SCF bands corresponding to the transmembrane and cleaved soluble forms of SCF (33 and 19 kDa, respectively; FIG. 10D). Compared with the corresponding forebrain tissue of uninjured mice, SCF protein levels increased as early as day 1 after injury, with the soluble form reaching maximum induction by day 5 and the membrane-bound form peaking 7 days after injury. The total amount of SCF also peaked at day 7 and then declined by day 12 after injury (FIG. 10E). Finally, we analyzed the distribution of SCF protein in the injured forebrain. SCF immunohistochemical staining was barely detectable in the uninjured control forebrains and only was weakly detectable in cortical layers I and II (FIGS. 11A and C). By contrast, prominent staining was seen in cells in layers II, III, IV, and V of the injured neocortex with extension up to the corpus callosum (FIGS. 11B and D). SCF staining was also observed in the SVZ of injured brain, but was not seen in the control brain (FIGS. 11E and F). In the contralateral, uninjured hemisphere, we observed some SCF staining in the SVZ and neocortex that was adjacent to the injured side. We did not, however, observe increased SCF expression in the areas of the contralateral uninjured brain at a distance from the site of injury. At a microscopic level it was clear that the highest level of SCF expression occurred in cells immediately adjacent to the site of direct injury, with a slow drop off in the level of expression as the distance increased away from the injury site (FIGS. 11G and H). We initially expected that the majority of the SCF would originate from inflammatory cells, specifically blood-borne monocytes and or microglia, however, only a minority of the SCF-expressing cells within the injured brains showed positive staining for microglia and monocytes markers, including lectin-RCA I, F4/80 and CD13 (FIGS. 12G and H). Likewise, the majority of GFAP-positive cells did not express SCF (FIGS. 12E and F). Instead there was intense SCF staining of the neuropil, indicating a predominantly neuronal expression of SCF. Immunohistochemical studies confirmed that the majority of SCF-expressing cells were positive for TUJ-1 and/or MAP2, consistent with neuronal SCF expression (FIG. 12A-D).

Expression of c-Kit on the NSPC and Responsiveness to SCF

Human fetal and mouse E14 NSPC populations from embryonic day 14.5 were expanded in serum-free media containing bFGF and EGF. These undifferentiated brain-derived NSPCs grew in neurospheres under our experimental conditions and avidly expressed nestin, a marker for NSPCs. In contrast, the cells showed negative staining for endothelia and mesenchymal differentiation markers. The true NSPC-like nature of our cells were confirmed by exposure of the cells to differentiating conditions, which resulted in expression of TUJ-1, GFAP, and CNPase in cells with morphologies consistent with cells of neuronal, astrocytic, and oligodendrocytic lineages, respectively.

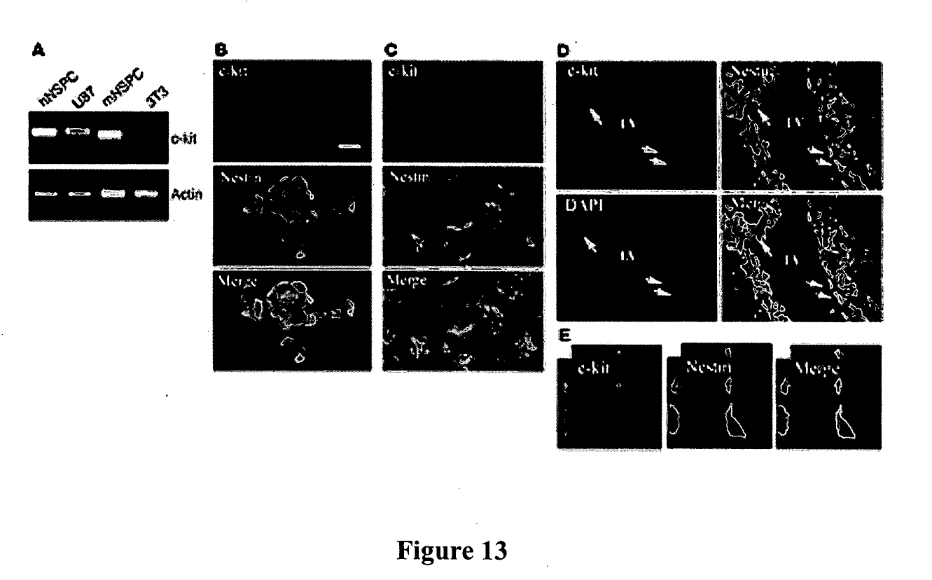

To more directly address the potential role of SCF on neural progenitor migration, we examined c-Kit (SCF receptor) expression and activation on the mouse NSPCs as well as human NSPCs. Semiquantitative RT-PCR revealed abundant c-Kit mRNA in the cultured mouse and human NSPCs (FIG. 13A). Immunohistochemistry confirmed the expression of c-Kit receptor in the cultured mouse and human NSPC (FIGS. 13B and C). In addition, we could readily identify a subpopulation of nestin-positive cells in the SVZ that were c-Kit positive (FIGS. 13D and E), indicating c-Kit expression on adult neural progenitors in vivo.

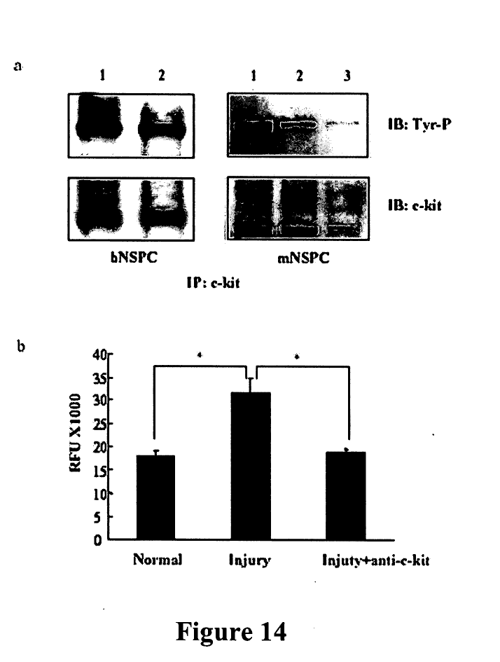

The binding of SCF to its receptor, c-Kit, is known to induce c-Kit autophosphorylation on tyrosine residues, resulting in activation of various downstream signaling pathways. In order to evaluate whether the c-Kit receptor was functional on neural progenitor cells, we stimulated the NSPC with 100 ng/ml of rmSCF followed by immunoprecipitation of cellular proteins using a c-Kit Ab. Immunoprecipitated proteins were then immunobloted using a phosphotyrosine Ab followed by reprobing of the blot with a c-Kit Ab, as described in Example 2. The results demonstrated that c-Kit proteins on NSPCs were strongly phosphorylated following SCF stimulation. Moreover, pretreatment of mouse NSPCs, with a specific c-Kit -blocking Ab (ACK 45) markedly decreased c-Kit autophosphorylation on mouse NSPCs (FIG. 14A). These data demonstrate that the c-Kit receptor is biologically active, as determined by tyrosine kinase activity, on neural progenitor cells.

Chemoattractive Effects of SCF on NSPC Migration in vitro

We next asked if the SCF upregulation in the injured brain was functionally relevant for neural progenitor migration. We established a Boyden chamber-based migration assay to quantitatively evaluate NSPC migration in vitro. Lysates taken from injured brains induced significantly more cell migration than did lysates from normal brains. Additionally, the increased cell migration seen following exposure of NSPC to injured brain lysates could be abolished by pretreatment of the NSPCs with the ACK45 c-Kit-blocking Ab (FIG. 14B). This migration inhibition did not occur after pretreatment with a control rat IgG.

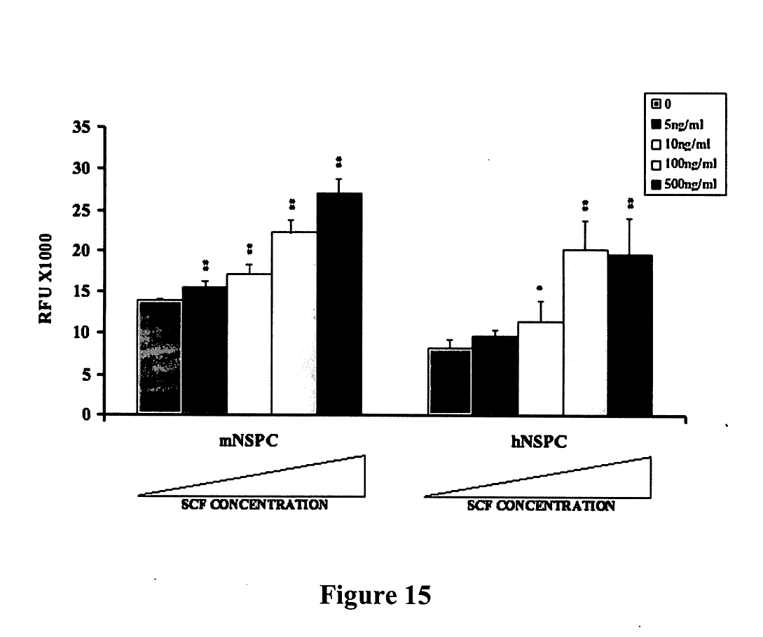

To directly evaluate the chemoattractant effects of SCF on neural stem cell migration, we used various concentrations (5-500 ng/ml) of purified rmSCF to induce NSPC migration in the Boyden Chamber migration assay. We observed that c-Kit mRNA was not detectable on the NIH 3T3 cells with RT-PCR (FIG. 13A,) and therefore we included NIH 3T3 cell in the Boyden chamber assay as a negative control. In the presence of 1% serum, NSPCs and NIH 3T3 demonstrated the same minimal nonspecific migratory response. The response to rmSCF, however, was quite different between these cell types; rmSCF did not induce migration of NIH 3T3 cells regardless of the concentration used. In contrast, rmSCF resulted a significant chemoattractive effect on NSPC in a dose dependent manner (FIG. 15). Statistically significant increases in cell migration were seen at SCF concentrations as low as 5 ng/ml for mouse NSPCs and at 10 ng/ml for human NSPCs. This experiment was repeated three times and similar results were obtained. These data demonstrate that SCF regulates NSPC migration in vitro.

Chemoattractive Effects of SCF on Neural Stem Cell Migration in vivo

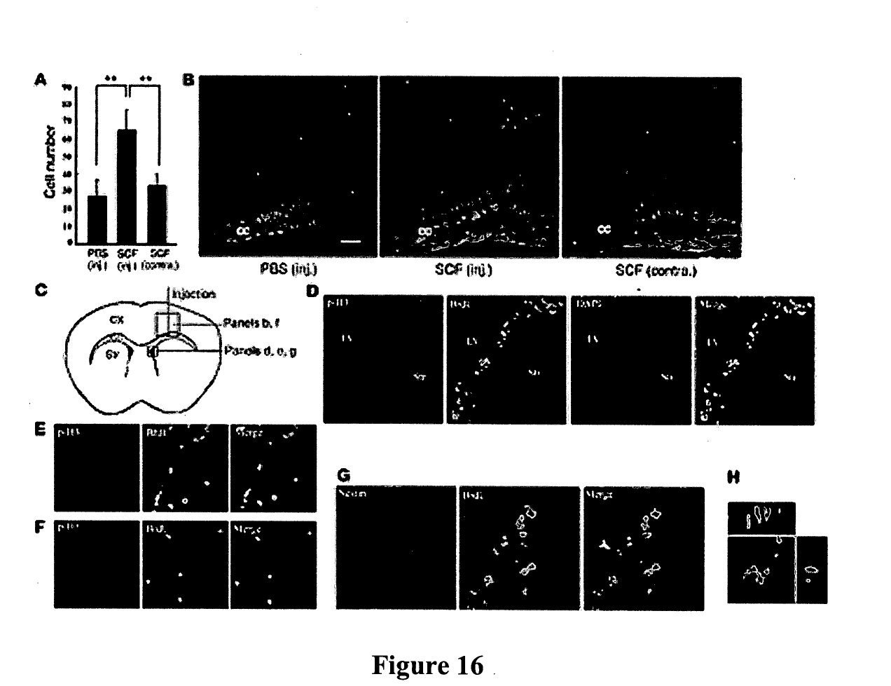

To test the effects of SCF on endogenous progenitor cell migration in vivo, we prelabeled endogenous dividing NSPCs with BrdU in SCID mice. Twenty hours after the last BrdU injection, several brains were processed for BrdU immunohistochemistry analysis as a day 0 control. The remaining animals were divided into two groups: those with intracerebral injection of rmSCF and those injected with the vehicle control (PBS). Treated animals were euthanized on day 7 after SCF or PBS administration. The brain sections were then evaluated for expression of BrdU, nestin, and phospho-histone H3 by immunohistochemistry and fluorescence confocal microscopy. The SCF-treated group had significant numbers of BrdU-positive cells in the SCF-injected area compared with both the contralateral side of the same brain and the PBS-injected animals (FIGS. 16A and B). There were approximately twice the number of BrdU-positive cells in the SCF-injected area (1.5-mm² section) compared to the contralateral side and 2.3 times the number of cells in the injected area in SCF-treated animals compared to PBS-treated animals (p<0.01). In contrast, the day-0 group animals, the majority of dividing cells were distributed within the SVZ, with only a few cells widely dispersed within the corpus callosum, cerebral cortex and striatum (FIG. 16D). Moreover, BrdU incorporation colocalized with the cell proliferation marker, phospho-histone H3, demonstrating that BrdU incorporation was a function of cell division rather than DNA damage (FIG. 16D-F). The majority of BrdU-positive cells expressed nestin, indicating that they were NSPCs (FIGS. 16G and H).

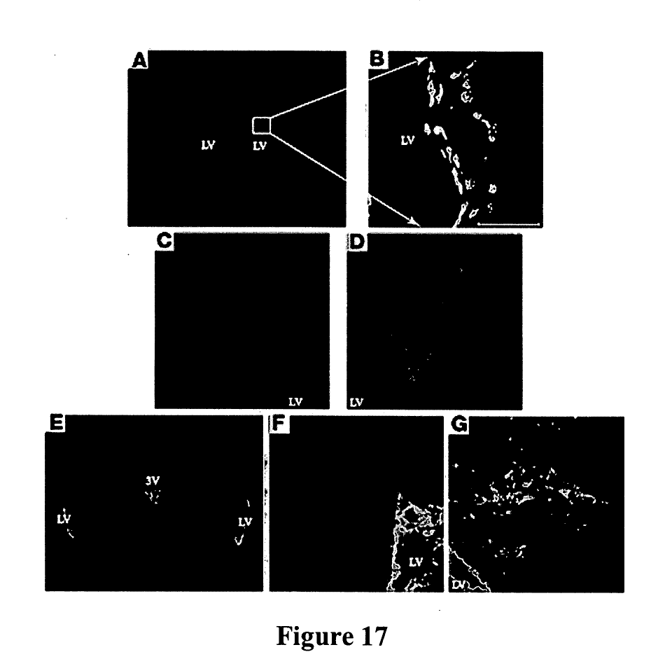

To clearly demonstrate that the BrdU-positive stem cells were SVZ stem cells that had migrated to the SCF injection site, rather than representing proliferation of a local population of stem cells, we injected DiI flurorescent dye or adenovirus expressing GFP into the cerebral ventricle of the mouse. As has been previously demonstrated (Clarke, D. L. et al. 1999 Cell 96:25-36), ventricular injection of DiI or GFP adenovirus enables one to track the progeny of NSPCs in the SVZ region. The ventricular injection of DiI or adenovirus expressing GFP was given prior to SCF administration. The injection of DiI was restricted to areas near ventricular zones (FIG. 17A), eliminating the possibility of diffusion of free dye into other areas of the brain, thereby ensuring the labeling of cells that were immediately adjacent to the ventricle (FIG. 17B). Following the injection of SCF, significant numbers of DiI labelled cells were found near the injection site of SCF. In contrast, few DiL-labeled cells were detected in the contralateral cortex of SCF injected animals or near the injection site of animals injected with PBS (FIGS. 17C and D). Similarly, GFP expression was initially confined to cells immediately adjacent to the ventricular system after the injection of GFP-expressing adenoviral vectors into the ventricles (FIG. 17E). After SCF injection, however, a large number of GFP-positive cells had migrated into the SCF-injected cortex (FIGS. 17F and G). Taken together, these ventricular labeling experiments clearly show that SVZ stem cells migrate into the SCF injected cortex.

### DISCUSSION

NSPCs have marked migratory abilities within the CNS under physiological and pathological conditions such as during embryonic nervous system development, adult neurogenesis, and injury response. Although there is a growing body of literature regarding the mechanisms responsible for NSPC migration during CNS development, little is known of the mechanisms that are operative in mediating NSPC migration during times of injury. Understanding of the signals responsible for NSPC migration to sites of brain and spinal cord injury could prove crucial for strategies attempting NSPC-mediated repair of such damage.

To begin to elucidate the signals involved in NSPC migration to sites of CNS injury, we constructed a “subtraction library” of injured brain tissue compared to control brain tissue. We identified more than 300 clones representing 196 unique genes that are differentially expressed in the injured tissue. In order to prioritize clones that might be most interesting to evaluate first, we made the assumption that a neural progenitor chemoattractant would likely be a membrane bound and/or secreted protein with the ability to diffuse within the extracellular space of the brain, given the pronounced effect of injury on NSPC migration at relatively large distances. Thus, we performed a bioinformatics screen of our clones, looking for genes that contained a predicted signal peptide. One of the most prominent clones represented in our subtraction and bioinformatics screen was that encoding SCF. Our data demonstrate that SCF mRNA is overexpressed in injured brain compared with normal brain by SSH analysis, custom cDNA microarray analysis and real-time quantitative RT-PCR. Correspondingly, SCF protein is also induced by injury.

A single SCF gene that maps to 12q22-24, encodes two different isoforms of the protein (Geissler, E. N. et al. 1991 Somat. Cell Mol. Genet. 17:207-214, Zsebo, K. M. et al. 1990 Cell 63:213-224). A longer 248 amino acid SCF isoform contains a proteolytic cleavage site at Ala 165 that, after cleavage, results in loss of the transmembrane region and a soluble truncated protein (Pandiella, A. et al. 1992 J. Biol. Chem. 267:24028-24033, Longley, B. J. et al. 1997 PNAS USA 94:9017-9021). Both SCF isoforms bind and activate the c-Kit receptor, although their effects on c-Kit signaling may be subtly different secondary to differences in receptor internalization (Jiang, X. et al 2000 EMBO J. 19:3192-3203). Specifically, the soluble c-Kit/SCF complex is rapidly internalized and degraded, resulting in transient tyrosine kinase activation of c-Kit, whereas the membrane bound SCF appears to prevent receptor internalization, resulting in more persistent receptor tyrosine phosphorylation (Kurosawa, K. et al. 1996 Blood 87:2235-2243; Miyazawa, K. et al. 1995 Blood 85:641-649; Ikuta, K. et al. 1991 Int. J. Cell Cloning 9:451-460). Loss-of-function mutations in SCF or c-Kit has demonstrated the importance of SCF/c-Kit pathway in hematopoiesis, gametogenesis, and melanogenesis. In contrast, activating mutations in c-Kit mediate transformation of hematopoietic stem cells, mast cells, and gastrointestinal stromal cells (Taylor, M. L. et al. 2001 Blood 98:1195-1199; Galli, S. J. et al. 1992 Ann. N.Y. Acad. Sci. 664:69-88; Mackenzie, M. A. et al. 1997 Dev. Biol. 192:99-107; Kunisada, T. et al. 1998 Development 125:2915-2923; Miller, S. C. et al. 1993 Nat. Immun. 12:293-301; Kunisada, T. et al. 1998 J. Exp. Med. 187:1565-1573). Recent reports have indicated that the SCF/c-Kit pathway is activated during injury and plays a protective role in selected injury models of non-neural tissue (Bone-Larson, C. L. et al. 2000 Am. J. Pathol. 157:1177-1186; Simpson, K. et al. 2003 Lab Invest 83:199-206). In addition to these effects on cellular proliferation, differentiation, survival and transformation, SCF can also induce migration of hematopoietic stem cells, melanoblasts, and mast cells (Kim, C. H. & Broxmeyer, H. E. 1998 Blood 91:100-110; Meininger, C. J. et al. 1992 Blood 79:958-963; Nilsson, G. et al. 1994 J. Immunol. 153:3717-3723; Gomperts, M. et al. 1994 Ciba Found. Symp. 182:121-134; Jordan, S. A. & Jackson, I. J. 2000 Dev. Biol. 225:424-436; Klein, A. et al. 2000 J. Immunol. 164:4271-4276; Drayer, A. L. et al. 2000 Br. J. Haematol. 109:776-784).

Our data are consistent with a previous report demonstrating that SCF expression is induced by brain injury in neurons surrounding the injury, as assessed by in situ hybridization and immunohistochemistry (Zhang, S. C. & Fedoroff, S. 1999 Acta Neuropathol. (Berl) 97:393-398). Additionally, a recent study has shown increased SCF levels in the medium from cerebral cortical cultures after hypoxic insult, indicating that SCF is also induced in other types of neural injury such as cerebral ischemia (Jin, K. et al. 2002 J. Clin. Invest 110:311-319). We have now extended these observations by demonstrating not only that SCF is overexpressed in neurons following traumatic injury in vivo but also that recombinant SCF can directly induce neural stem cell migration in vitro and in vivo. These data, therefore, indicate a role for SCF in mediating NSPC migration to areas of acute brain injury. Of interest is a previous report indicating that the brain predominantly produces the soluble form of SCF (Huang, E. J. et al. 1992 Mol. Biol. Cell 3:349-362). Our Western blot data, however, clearly demonstrate that although the soluble form of SCF is predominant, membrane bound SCF is also increased in the traumatized brain. Given the differences in the degree and kinetics of c-Kit activation between the two different SCF isoforms, it is tempting to speculate that the production of both the soluble and membrane form of the cytokine more readily allows for the establishment of a chemotaxis gradient toward the sight of injury.

In addition to demonstrating the direct migratory effects of SCF on NSPCs, we have also demonstrated the presence of c-Kit on NSPCs, the resultant tyrosine phosphorylation of the receptor after the exposure to SCF, and the presence of activated receptor on progenitor cells in the SVZ of the lateral ventricle and subgranular zone of the hippocampal dentate gyrus. These data are consistent with those of Jin and co-workers, who demonstrated c-Kit expression in neuroproliferative zones in the adult mammalian brain following ischemic injury (Jin, K. et al. 2002 J. Clin. Invest 110:311-319). The signal transduction pathways operative in NSPC migration following c-Kit activation remain to be fully elucidated, although recent evidence indicates that c-Kit mediated mast cell migration is dependent on phosphoinositide-3 kinase (PI-3kinase) signaling (Li, L. et al 2002 Mol Cell Neurosci 20:21-9). Recent work has demonstrated that mouse NSPC heterozygous for phosphatase and tensin homolog deleted on chromosome 10 (PTEN) have increased migratory properties and that conditional PTEN knock-out mice have disorganization of brain neuronal architecture, probably due to precursor migratory errors. These data are consistent with a central role for PI-3Kinase in NSPC migration since PTEN is a negative regulator of components of PI-3Kinase signaling (Li, L. et al. 2002 Mol Cell Neurosci 20:21-9, Li, L. et al. 2003 J. Cell Biochem 88:24-8). Finally, recent data indicate a role for Slug, a member of the Snail zinc-finger family of transcription factors, in SCF-mediated migration of hematopoietic and gamete stem cells (Perez-Losada, J. et al. 2002 Blood 100:1274-1286). Although further studies are clearly needed to precisely define the role of PI-3Kinase and Slug in c-Kit induced NSPC migration, our data clearly demonstrate that the. SCF/c-Kit pathway is activated within the brain after both traumatic and ischemic injury and contributes to NSPC migration to those sites of injury.

Although both SCF expression in neurons and c-Kit activation on NSPC after injury have been described previously, this is the first report to our knowledge that has made the functional connection between neuronal-expressed SCF and induction of NSPC migration to the site of injury. Despite the known migratory effects of SCF on other types of stem cell populations, it is empirical that SCF has such effects on NSPC. We do not at this time understand the significance of injury-induced SCF-mediated neural stem cell migration to sites of injury, although it is tempting to speculate that recruitment of NSPCs is part of an injury repair process. Whether SCF/c-Kit mediated recruitment of NSPC to sites of injury actually accomplishes this goal, what other molecules are operative in this process, and whether SCF is playing an additional role in injury-induced neurogenesis and stem cell differentiation within the area of injury are important questions that will require additional study. Irrespective of the physiological role of SCF in the brain, the elucidation of SCF as a potent NSPC migratory factor opens up the opportunity to utilize recombinant or genetic vector-derived SCF as a chemotactic agent to induce NSPC recruitment to specific areas of the central nervous system for cell-based therapeutic interventions.

### EXAMPLE 1

Cell Culture

Primary ECs (Clonetics, Walkersville, Md.) and human astrocytes (Sciencell Research Laboratories, San Diego, Calif.) were used at passage 8 or earlier. HBMEC (Sciencell Research Laboratories) was used at passage 1 or 2. All malignant glioma cells except for 1321N (Europe collection of cell cultures, Sigma-Aldrich Corp., St. Louis, Mo.), bone marrow neuroblastoma cell, SK-N-SH, and NIH 3T3 were purchased from American Type Culture Collection (Manassas, Va.). All cells were passaged and cultured in conditions recommended by their suppliers.

Thymidine Incorporation

After ECs (3×10³/well) were adhered to 96-well gelatin-coated plates (BD Discovery Labware, Bedford, Mass.), the EC growth medium was replaced by SCF (1.10 ng/ml) (R&D Systems, Minneapolis, Minn.) suspended in treatment medium (basal medium with 0.1% BSA) for an additional 48 hr (HUVEC and HMVEC-d) or 72 hr (BMVEC-b) with renewal of medium at 24 hr intervals. ECs were cultured for 4 hr in 0.25 μCi of [³H]thymidine (Amersham Pharmacia Biotech, Piscataway, N.J.) at the end. High molecular mass [³H]-radioactivity was precipitated and counted.

EC Proliferation

The HBMEC and HUVEC were seeded on 48-well and 24-well plates (poly-L-lysine-coated plates for HBMEC) at 5×10³ or 2×10⁴ per well, respectively. After cells adhered to the well (6 hr), growth medium was replaced by CM. The CM collected from U251 glioma cell cultures were pretreated with SCF or VEGF neutralized antibodies (R&D systems) at the indicated concentrations for 1 hr at 37° C. before being added to the HUVEC. After 48 hr culture in the treated media, dead floating cells were washed away, and attached living cells were trypsinized and counted with hematocytometer.

EC Differentiation Assay

After the Matrigel (BD Biosciences) formed a solid gel in 24-well plates, serum-starved BMVEC-b cells were seeded on the gel surface at 10⁵ cells per well and cultured in treatment medium in the presence or absence of SCF (20 ng/ml). Primary HBMEC were placed on the gel directly at 24×10³ per well. Tubular formation was observed using an inverted phase contrast microscope, and images were captured at 4 and 18 hr after cell seeding. Each experiment was repeated two or three times.

Wound Healing Assay

The BMVEC-b (3×10⁵/well) was seeded on collagen I-coated plates (24-well). After cells adhered to the plates (3 hr), growth medium was replaced by SCF and β-FGF (R&D Systems) or vehicle in treatment medium. After 6 hr of incubation, confluent monolayers of BMVEC-b were wounded with a pipette tip and incubated in the same condition. The cells were fixed with 4% paraformaldehyde 18 hr after being wounded, and images were captured. The cells that had migrated across the edge of the wound were observed microscopically (magnification, ×20) and counted in three fields for each well. All assays were repeated three times in duplicates or triplicates.

Flow Cytometry Analysis

Cells were incubated with Cy-Chrome conjugated anti-human c-Kit (CD117) antibody or PE-Cy5 mouse IgG1 as a negative control (BD Pharmingen, San Diego, Calif.), respectively. Dead cells were excluded by propidium iodide staining (Sigma Chemie), and expression of c-Kit on the cell surface was analyzed by FACS (Becton Dickinson, San Jose, Calif.). For the detection of total c-Kit including that in the cytosol, permeabilization was performed with BD cytofix/cytoperm solution (Kit from BD Biosciences) for 15 min prior to the incubation with c-Kit antibody.

Immunoprecipitation and Immunoblotting

Confluent-grown ECs were treated with 50 ng/ml of SCF for 5, 10, 15, and 30 min after 24 hr of serum starvation. The ECs were collected with RIPA lysis buffer (Upstate, Charlottesville, Va.) containing 50 mM NaF, 1 mM Na₃VO₄, 50 mM PMSF, and Protease Inhibitor Cocktail (Roche). The antibodies were purchased from Cell Signaling Technology, Inc. (Beverly, Mass.) or as mentioned otherwise. All the primary antibodies were used in a 1:1000 dilution except for the anti-SCF antibody, which was used in a 1:2000 dilution. The primary antibodies were anti-phospho-c-Kit, anti-phospho-MEK1/2 and anti-MEK1/2, anti-phospho-p44/42 MAPK and anti-p44/42 MAPK, anti-phospho-SAPK/JNK and anti-SAPK/JNK, anti-phospho-p38 MAPK and anti-p38 MAPK, anti-phospho-Akt and anti-Akt, anti-phospho-mTOR and anti-mTOR, anti-phospho-4E-BP1 and anti-4E-BP1, anti-SCF and anti-VEGF (Chemicon, Temecula, Calif.), and anti-β-tubulin (Sigma). Re-blotting was done after membranes were stripped by re-blot kit (Chemicon). Tie2 detection in Matrigel plugs from the in vivo Matrigel assay was carried out by immunoprecipitation and immunoblotting. The gel plugs were carefully separated from muscle and skin and homogenized with RIPA lysis buffer. The resulting total tissue lysates (40 mg of protein) were immunoprecipitated with Tie2 antibody (Santa Cruz Biotechnology, Inc., Santa Cruz, Calif.) and then analyzed by immunoblot with Tie2 antibody (Zadeh et al. 2004 Am J Pathol 162:467-476).

ELISA

Cells were seeded at 80% of confluence, and the growth medium was changed 24 hr later to basal medium containing 0.1% BSA after washing with PBS. Forty-eight hours later, the CM were collected, and supernatants were obtained by centrifuge at 2K RPM for 5 min. SCF in the CM was measured using the human SCF ELISA kit (R&D System) in triplicate following the protocol provided by the manufactory and normalized by the total protein levels in the media of each individual sample.

Microarray Analysis

All gliomas (grade II, III, and IV) were pathologically diagnosed according to WHO standard. All tumors and nontumor brain tissues of epilepsy were obtained from surgery patients and frozen immediately after operation (clinical protocol approved by the NCI IRB committee with informed consent obtained from all subjects). The frozen samples were disrupted and homogenized with TRIZOL (Invitrogen Corp., Carlsbad, Calif.), and the total RNA isolated from each sample was further purified using RNeasy Mini Kit (Qiagen, Valencia, Calif.). The integrity and quality of the RNA met the quality requirements for Human Genome U133 Plus 2.0 arrays (Affymetrix, Inc., Santa Clara, Calif.) recommended by the company. All experimental procedures for labeling, array hybridization, and scanning were performed using protocols recommended by Affymetrix. CEL files were generated by Affymetrix GCOS 1.2 software, and the present/absent calls were defined with global scaling to target value of 500. By dChip software (Li and Wong, 2001 Proc Natl Acad Sci USA 98:31-36), the CEL files were normalized to a median-intensity array, and model-based expression values were calculated using PM/MM difference model. Based on the latest annotation from Affymetrix NETAFFX service, four probe sets for SCF (KITLG) gene were present in each chip. The signal intensities of each probe set were used for analyzing SCF (KITLG) expressions. The significance in differential SCF (KITLG) expressions in different grades of gliomas versus human nontumor brains was determined using log2-transformed expression values by standard unpaired two-tailed Student's t test of two groups without assuming equal variance between groups. The microarray data have been submitted to the Gene Expression Omnibus (GEO) public database at NCBI, and the accession number is GSE4290.

Real-Time RT-PCR

RNA isolation and cDNA synthesis were performed as described above. A human probe set covering exon 8 of SCF gene was purchased from Applied Biosystems (Foster City, Calif.). Isoform-specific probe sets were selected in the connecting region of exons 5 and 7 (for mSCF) and in exon 6 (for sSCF) (Table 2). Two-step real-time PCR was performed in duplicates or triplicates. Data were analyzed on the basis of threshold cycle values of each sample and normalized with β-actin.

TABLE 2

SCF probe sets for real-time PCR

| | |
|---|---|
| primer | probe |

| | | | |
|---|---|---|---|
| mSCF | up | GATTGTGTGGTTTCTTCAACAT | AGAAAGGGAAGGCCA |
| | | TAAGTC | (SEQ ID NO: 2) |
| | | (SEQ ID NO: 1) | |
| | down | AGTATAAGGCTCCAAAAGCAAA | |
| | | GC | |
| | | (SEQ ID NO: 3) | |
| sSCF | up | GTTGCAGCCAGCTCCCTTAG | CAGTAGCAGTAATAGGAA |
| | | (SEQ ID NO: 4) | (SEQ ID NO: 5) |
| | down | AGAAAACAATGCTGGCAATGC | |
| | | (SEQ ID NO: 6) | |

Expression Vector and Transduction

SCF cDNA was synthesized by RT-PCR and subcloned into the pLenti6/V5-DEST vector (Invitrogen Corp.) in the antisense orientation following the supplier's protocol. The 293FT package cell was cotransfected with the expression vector and ViralPower Packaging Mixture (Invitrogen Corp.). The supernatants containing infectious virus particles from transfected 293FT were used to transduce U373 and U87 glioma cells. The stably transduced U373 and U87 cell lines were selected with 5 ug/ml of blasticidin for over 2 weeks, and the amount of SCF protein expression was assayed by immunoblot. The retroviral expression vector pSM2 with the SCF shRNA insert and the LinX retroviral packaging cells were purchased from Open Biosystems (Hunstsville, Al.). The retrovirus was obtained from plasmid transfected LinX cells and was used to transduce U251 cells following the manufacturer's protocol. The stable shRNA expression cells were selected with puromycin at 1 ng/ml for over 2 weeks, and downregulation of SCF was verified by immunoblot.

In vivo Angiogenesis Assay and Intracranial Tumor Model

All adult mice (6-8 weeks of age) were from NCI-Fredrick, and the animal experiments were conducted on an animal experimentation protocol approved by the NCI's animal protection committee (the “ACUC”). For the in vivo angiogenesis assay (Capogrossi and Passaniti, 1999 “An in vivo angiogenesis assay to study positive and negative regulators of neovascularization.” in A. H. Baker, Editor, Methods in Molecular Medicine, Vascular Disease: Molecular Biology and Gene Therapy Protocols, Humana Press Inc., Totowa, N.J., pp. 367-384), Matrigel with 150 ng of SCF and controls or 5×10³ cells was injected into a SCID mouse, and the plugs with skin and muscle were collected 2 weeks later. For the intracranial implantation of glioma cells, stereotactic surgery was performed (1 mm anterior to the bregma and 2.5 mm lateral to the midline) using athymic nu/nu mice under anesthesia. U373/vector or U373/as-SCF (10⁶ cell) were suspended in 5 μl of HANK and injected into the caudate nucleus. For immunohistochemical analysis, mice were sacrificed 4 weeks later, and brains were carefully recovered after perfusion.

Immunohistochemistry

The fixed brains and Matrigel plugs were prepared for paraffin sectioning and immunohistochemical analysis. The human GBM tissues were obtained from surgery patients following the clinical protocol approved by the NCI IRB committee with informed consent obtained from all subjects. After being deparaffinized, the sections were processed for immunohistochemistry with the following primary antibodies: anti-vWF (1:500 dilution), anti-c-Kit (1:200 dilution), anti-CD31 (1:20 dilution), and anti-SCF (1:100 dilution), respectively, All of the primary antibodies were purchased from Dako Corp. (Carpinteria, CA) except for the anti-SCF antibody, which was bought from Chemicon. Visualization of antibody binding was performed by a FITC-conjugated second antibody (1:500 dilution; Molecular Probes, Eugene, Oreg.) or by the second antibody tagged with HRP (1:1000 dilution; Dako) following reaction to DAB (Molecular Probes) or AEC (Sigma).

In situ Hybridization

The DIG-labeled probe hybridized to nucleotides 848-895 of the mouse SCF mRNA sequence (NM_{—}013598 in GenBank) was obtained from GeneDetect company (Aucklank, New Zealand). After they were deparaffinized, mouse sections were permeabilized with target retrieval solution (Dako) and hybridized with sense or antisense DIG-labeled probe at 37° C. for 18 hr. After stringent washing, the hybridized signals were detected by tyramide signal amplification system and visualized with DAB (Dako).

Statistical Analysis

Student's t test was used to determine statistical significance in comparisons. Survival curves were plotted with Kaplan-Meier method and compared using log-rank test. p<0.05 was considered significance. Survival trends of grade IV glioma patients (n=64) were analyzed relative to SCF mRNA levels. The actin normalized SCF levels ranging from 0.64- to 10.76-fold were determined by real-time PCR; levels less than 1.8 were grouped as low SCF, and those greater than 1.8 were classified as high SCF. In the figures, columns and bars show the mean and SEM, respectively.

### EXAMPLE 2

Animals and “Freeze” Injury

All mice (NCI-Fredrick, Fredrick, Md., USA) were handled following Institutional guidelines (National Institutes of Health ethics guidelines for use of animals in research). Stereotactic surgery was performed with SCID male mice (6-8 weeks of age) sedated under anesthesia (ketamine and xylazine at doses of 80 and 10 mg/kg body weight, respectively by intraperitoneal administration). As schematically illustrated in the FIG. 10A, the skull was opened 1 mm anterior to the bregma and 2.5 mm lateral to the midline with the dental drill leaving the dura intact. The “freeze” brain injury was produced by insertion of a Hamilton syringe pre-cooled in liquid nitrogen through a cranial hole to a depth of 2 mm below the dural surface. The needle was kept in this position for 30 sec. This procedure was repeated five times in each animal. The animal's body temperature was kept within physiological range during and after the surgery. The animals were euthanized at 5 days after injury by CO₂ inhalation and their brains were rapidly dissected on an ice-cold board. The dorsal forebrain ipsilateral and contralateral to the site of injury was rapidly dissected out (illustrated in FIG. 10A) and stored at −70° C. for RNA extraction. The entire dorsal forebrain from animals with bilateral injury was collected for Western blot and the Boyden chamber migration assay. In these studies, the corresponding brain areas from naive animals were used as the control.

Cell Culture

Human NSPCs (Clonetics, Walkersville, Md., USA) and mouse NSPCs, isolated from the forebrain of embryos at 14.5 day of gestation, were cultured in neural basal medium supplemented with B27 (Invitrogen Corporation, Carlsbad, Calif., USA), 20 ng/ml of bFGF and 20 ng/ml of EGF (R&D systems, Minneapolis, Minn., USA), 0.5 μM glutamine, and appropriate antibiotics, as described previously (Carpenter, M. K. et al. 1999 Exp. Neurol. 158:265-278). The cells grew in a 6-well plate as nonadherent cells and were prevented from attaching to the plates by periodic gentle agitation of the plates each day. EGF and bFGF were added every other day and culture medium was changed weekly until neurospheres became visible. The spheres were passaged by enzymatic and mechanical dissociation every 7-10 days and were reseeded as single cells into growth medium at a density of ˜100,000 cells/ml. The cells that adhered to the plastic and began to extend processes were removed from the well and were not cultured in subsequent passages. U-87, a cell line derived from a malignant glioma, and NIH 3T3 were purchased from ATCC (Manassas, Va., USA) and cultured in the conditions recommended by the company.

Boyden Chamber Assay

The effects of SCF on NSPC migration were determined by a modified Boyden chamber assay as described previously (Sun, L. et al. 2001 Cancer Res. 61:4994-5001). A 96-well cell migration kit was utilized (Chemicon, Temecula, Calif., USA) and each well was separated into two chambers by a membrane with 8-μm pores. The cells were briefly trypsinized and adjusted to 4×10⁵ cells/ml in 1:20 dilution of B27 medium without growth factors. Some cells were pretreated with a c-Kit-blocking antibody (ACK45, PharMingen, San Diego, Calif., USA) for 30 min at room temperature. Migration of NIH 3T3 cell was also assayed as a control. Recombinant mouse SCF (rmSCF) (R&D systems, Minneapolis, Minn., USA) was dissolved in the same medium and adjusted to the various concentrations. After different concentrations of SCF or brain tissue lysates were added to the lower chamber as attractants, 100 μl of the NSPCs (4×10⁴) were applied to the upper chamber on the top of membrane. A group of control chambers without rmSCF was also included. After 4 hours of incubation at 37° C., the migratory cells on the bottom of the insert membrane were dissociated from the membrane by incubation with cell detachment buffer. These cells were subsequently lysed and stained by the CyQuant GR dye (Chemicon), which exhibits strong fluorescence enhancement when bound to cellular nucleic acids, and measured using a fluorescence plate reader with a 480/520-nm filter set.

SSH Library

Total RNA was isolated from either the injured or contralateral dorsal forebrain from 60 mice using Trizol (Invitrogen, Carlsbad, Calif., USA) and purified with an RNeasy kit (Qiagen, Valencia, Calif., USA). The poly(A)RNA was subsequently isolated from purified total RNA with a poly(A)RNA isolation kit (Qiagen, Valencia, Calif., USA). Complimentary DNA (cDNA) was synthesized from 2 μg poly(A)RNA using the SMART PCR cDNA Synthesis Kit and subtractive hybridization was performed with the CLONTECH PCR-selective cDNA Subtraction Kit (BD Biosciences, Palo Alto, Calif., USA) based on the protocols suggested by the company. The “tester” cDNA came from injured brain, whereas the “driver” pool was from uninjured forebrain. After adaptor ligation, the “tester” cDNA pool was then hybridized with “driver” cDNAs at a ratio of 1:20 for selection of transcripts specifically upregulated in the injured hemisphere. After hybridization, suppression PCR using primers specific for the “tester” PCR adaptors selectively amplified differentially expressed transcripts for 15 cycles. The amplified cDNAs were subsequently cloned into the T/A cloning vector pGEM-T easy (Promega, Madison, Wis., USA). and about 500 colonies were obtained in total.

Custom cDNA Microarray

To confirm the cDNA clones of the SSH library contained the upregulated transcripts induced by the injury; custom cDNA microarrays were constructed and used to screen the library. First, individual colonies from the SSH library were grown overnight at 37° C. in Luria broth medium containing 50 μg/ml of ampicillin. After the plasmids were isolated, the inserts were cut out by EcoRi restriction enzyme and were analyzed by electrophoresis on a 1% agarose gel. Inserts within the clones (˜85% of total) were then amplified by PCR. The PCR products were then “dot-blotted” onto GeneScreen Plus hybridization transfer membranes (Perkin Elmer Life Sciences Inc. Boston, Mass., USA) in duplicate by S&S Minifold I Dot-Blot System (Schleicher & Schuell, Dassel, Germany). In addition to the SSH library clones, each blot also contained actin and GAPDH as controls. cDNA targets were then labeled and hybridized to the filter. The poly(A)RNA from brain 5 days after injury and uninjured brain was “reverse-transcribed” into cDNA and was amplified for 10 cycles with SMART PCR cDNA Synthesis Kit as described above. Purified cDNA probes (100 ng per blot) were labeled with fluorescence by the random primer method using Gene Images Random Primer Labeling and Detection System (Amersham Pharmacia Biotech, Piscataway, N.J., USA). Each duplicated membrane was hybridized with the injured or uninjured brain-derived probes at 60° C. overnight. The subsequent washing and antibody-mediated detection was identical to the protocol recommended by the company. Finally, the hybridized membranes were laid next to each other, exposed to a Kodak X-ray film (Eastman Kodak Co., Rochester, N.Y., USA), and developed simultaneously.

DNA Sequencing and Sequence Analysis

Our custom cDNA microarray screen confirmed the presence of over 300 overexpressed clones in the SSH library. These clones were then sequenced using the ABI PRISM Dye Terminator Cycle Sequencing Ready Reaction kit (Perkin Elmer Life Sciences) with the T7 promoter primer on an ABI 373 automated sequencer (Applied BioSystems, Foster City, Calif., USA). Database homology searches for the cDNA sequence and deduced protein sequences were performed by a BLAST program using the DNA and expressed sequence tag DNA (EST) databases at the National Center for Biotechnology Information (ncbi.nih.gov/BLAST website). From the 300 clones, 196 unique genes were identified to be upregulated in the injured area of the forebrain.

RT-PCR and Real-Time PCR

The reverse transcription reaction and PCR were performed as previously described (Sun, L. et al. 1998 FEBS Lett. 441:392-396). The c-Kit primers for mouse: 5′-CCATGTGGCTAAAGATGAAC-3′ up-stream (SEQ ID NO: 7); and 5′-ACTGCTGGTGCTCGGGTTTG-3′ down-stream (SEQ ID NO: 8); and for human were: 5′-TATACAACCCTGGCATTATGTCC-3′ up-stream (SEQ ID NO: 9); and 5′-TGCGAAGGAGGCTAAACCTA-3′ down-stream (SEQ ID NO: 10). For real time RT-PCR analysis, SCF probes were selected in the connecting region of two exons: 5′-ACTCTAGCGTGTAAATC-3′ (SEQ ID NO: 11); the up-stream primer was 5′-GAAGTCAGTCTTTTCCCTTGACAGT-3′ (SEQ ID NO: 12), and the down-stream was 5′-GCATGTCACATTATACTATTGCAAACA-3′ (SEQ ID NO: 13). The real time PCR reaction was performed by the ABI PRISM 7900HT Sequence Detection System (Applied BioSystems) in triplicate and data were analyzed on the basis of threshold cycle values of each sample and normalized with 18S RNA.

Immunoprecipitation and Immunoblot

Injured dorsal forebrains were dissected 24 hours, 3 days, 5 days, 7 days and 12 days after injury and were homogenized in a lysis buffer containing 50 mM Tris-HCl (pH 8.0), 120 mM NaCl, 0.5% Nonidet P-40, and Protease Inhibitor Cocktail (Roche, Indianapolis, Ind., USA). Brain tissue was kept on ice for 30 min and supernatants were collected after centrifugation. The brain lysates were separated by a 4-12% polyacrylamide gel electrophoresis (Invitrogen) and transferred to the membranes. The filters were blotted with rabbit polyclonal anti-SCF antibody (1:50 dilution; Chemicon, Temecula, Calif., USA). The NSPCs were treated or not treated with 100 ng/ml of rmSCF or were left untreated for 10 min and then were washed with ice-cold PBS. Some mouse NSPCs were treated with rat monoclonal c-Kit blocking antibody, (ACK4; PharMingen, San Diego, Calif., USA) for 30 min at room temperature before SCF stimulation. The NSPCs were resuspended in a protease inhibitor mixture containing radioimmunoprecipitation (RIPA) buffer (1% NP-40, 0.5% Sodium deoxycholate, 0.1% SDS, 0.1 mg/ml PMSF, 0.1 mM NaVO₄ in PBS). The cell lysates were pre-cleared with protein A-Sepharose (Amersham Pharmacia Biotech, Piscataway, N.J., USA), incubated with rabbit polyclonal antibody to c-Kit (Clone H-300, Santa Cruz, San Diego, Calif., USA), and collected on protein A-Sepharose beads. After being washed and eluted, the immunoprecipitates were separated by electrophoresis through 7% polyacrylamide gels. After transfer, filters were hybridized with anti-phosphotyrosine antibody (1:1,000 dilution; clone PY99, Santa Cruz) and visualized with a peroxidase-conjugated secondary antibody. After detection of SCF or a phosphotyrosine within c-Kit, the membranes were stripped and re-blotted with beta-tubulin antibody (Santa Cru Biotechnology) or with anti-c-Kit (H-300, Santa Cruz Biotechnology).

Immunocytochemistry

Cells and brain sections were processed for immunocytochemistry as described previously (Lee, J. et al. 2002 Neoplasia. 4:312-323). Briefly, brains were fixed in 4% phosphate-buffered paraformaldehyde (PFA) after transcardial perfusion and processed for paraffin sections or floating sections. For paraffin sections, brains were embedded in paraplast and cut into coronal sections (10-μm in thickness) on a rotary microtome. After being deparaffinized, the sections were processed for immunohistochemistry. For floating sections, perfused brains were soaked in 30% sucrose overnight, frozen in isopentane cooled by dry ice, and cut on a cryostat into sections 30 μm in thickness. The NSPCs were cultured on laminin coated glass wells and were fixed in 4% PFA for 20 min at room temperature. Cells and brain sections were processed for immunohistochemistry using the following primary antibodies: rat monoclonal anti-BrdU (1:50 dilution; Accurate Chemical, Westbury, N.Y., USA); rabbit polyclonal anti-SCF (1:100 dilution; Chemicon) and mouse monoclonal anti-nestin (1:400 dilution; Chemicon); mouse monoclonal anti-glial fibrillary acidic protein (anti-GFAP) (1:400 dilution; Sigma-Aldrich), mouse monoclonal anti-microtubule-associated proteins 2 (anti-MAP2) (1:,000 dilution; Sigma-Aldrich); rat monoclonal anti-CD13 (1:100 dilution; BD PharMingen); rabbit polyclonal anti-c-Kit (1:100 dilution; H-300; Santa Cruz); mouse monoclonal anti-beta-tubulin type III (anti-TUJ-1) (1:2,000 dilution; Covance research products,. Berkeley, Calif., USA); biotinylated lectin ricinus communis agglutinin I (RCA I) (1:1,000 dilution, Vector Labs, Burlingame, Calif., USA); and rabbit polyclonal phospho-histone-H3 (1:200 dilution, Upstate, Lake Placid, N.Y., USA). In order to prevent nonspecific binding of antibodies, cells or sections were treated with 5% normal goat serum in PBS with 1% BSA. The specificity of immunolabeling was verified in all experiments by controls in which the primary antibody was omitted. Secondary antibodies tagged with Alexa Fluor 488 and 594 (Molecular Probes, Eugene, Oreg., USA) were used for visualization at a 1:500 dilution of 1:500 for each. For dual and triple staining, the primary antibodies used in one section were from different species and single-antibody staining was performed to ensure specificity of staining. Specimens were examined on Zeiss LSM 510 confocal imaging system (Zeiss, Heidelberg, Germany) for immunofluorescence. Individual optical sections (optical depth, less than 0.1 μm) were obtained for different fluorogens and stacked optical sections were merged using Maximum Projection Software (Zeiss). For quantitative immunocytochemical data, Abercrombe's correction was used to approximate the number of cells with positive staining per examined area and a percentage per total cells within the area. Data were expressed as means±SEM.

BrdU Labeling and Intracerebral Stereotacfic Injections

For labeling of neural progenitors in vivo, BrdU (Roche) was administrated intraperitoneally twice a day, at 8-hour intervals, for 14 days (50 mg/kg body weight). Twenty hour from the last BrdU injection, several animals were killed and their brains were sectioned for BrdU immunostaining. Intracerebral injections were administered to the remaining animals. Stereotaxic surgery was performed as described in the “freeze” injury, and the injections were given at the position of 1 mm anterior to the bregma, 1.5 mm lateral to the midline, and 2.5 mm ventral to the dura. Mice received a suspension of rmSCF (3 μg in 3 μl of PBS; R&D systems) or PBS through a Hamilton syringe. Each injection took 15 min. Seven days from the intracranial injection, the animals were killed and the brains were processed for BrdU immunohistochemical analysis. For ventricular injection of DiI (1,1′-dioctadecyl-6,6′-di(4-sulfophenyl)-3,3,3′,3′-tetrametylindocarbocyanine; Molecular Probes) or adenovirus expressing GFP, 2 μl of 0.2% DiI in DMSO or 2 μl of replication incompetent adenovirus (10⁸ plaque-forming units per μl) (He, T. C. et al. 1998 PNAS USA 95:2509-2514) was injected 0.5 mm posterior and 0.7 mm lateral to bregma, and 2 mm below the dura matter into the lateral ventricle. Three days after ventricular injection, SCF was injected into the left hemisphere as described above. Seven days after SCF injection, the brains were sectioned and analyzed for DiI or GFP distributions.

### EXAMPLE 3

We previously found that injury-induced neuronally-produced SCF attracted neural stem cell migration toward the injury side, which could be important for damage repair. For the survival of neural stem cells and further functional incorporation into the local site, the injured tissue also has to generate a new vascular supply for gas exchange, cell nutrition, waste disposal, and damaged tissue absorption. Our recent work demonstrated that SCF is an important pro-angiogenesis factor and plays a critical role in the brain microvascular endothelial cell proliferation and tube formation.

SCF Stimulated Angiogenesis in vivo

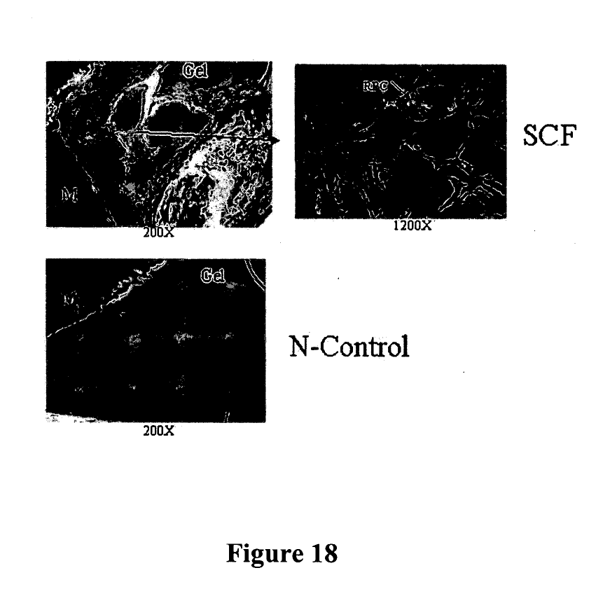

Recombinant mouse SCF (rmSCF) was used at 50 ng/ml in an in vivo angiogenesis assay. The matrigel plug containing SCF or PBS control was placed subcutaneously in SCID mice for 7 days, and the blood vessel formation within the gel was examined by H&E staining. As showed in FIG. 18, blood vessels can be seen in the SCF containing matrigel, which is obviously abundant compared with the PBS control (N-Control). In the higher magnification figure on the right side, the red blood cells inside of the newly formed vessel are indicated by an arrow.

SCF Stimulated Brain Microvascular Endothelial Cell (bMVEC-B) DNA Synthesis

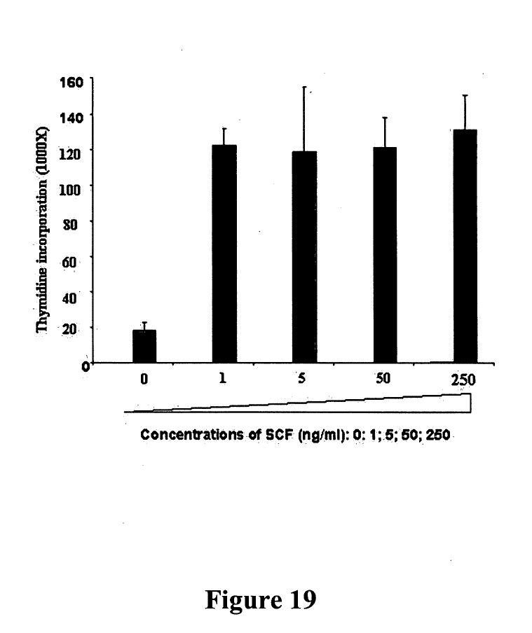

To specifically confirm the pro-angiogenic effects of SCF in the brain, bMVEC-B cells were used. The cells were cultured in the presence of different concentrations of rmSCF (1 ng/ml to 250 ng/ml) or 0.1% BSA only medium for 72 hours. The rate of DNA synthesis was measured by thymidine incorporation. FIG. 19 shows that SCF strongly stimulated bMVEC-B DNA synthesis and bMVEC-B responded to the SCF stimulation at concentrations as low as 1 ng/ml.

SCF Enhanced bMVEC-B Cell Tube Formation on Matrigel

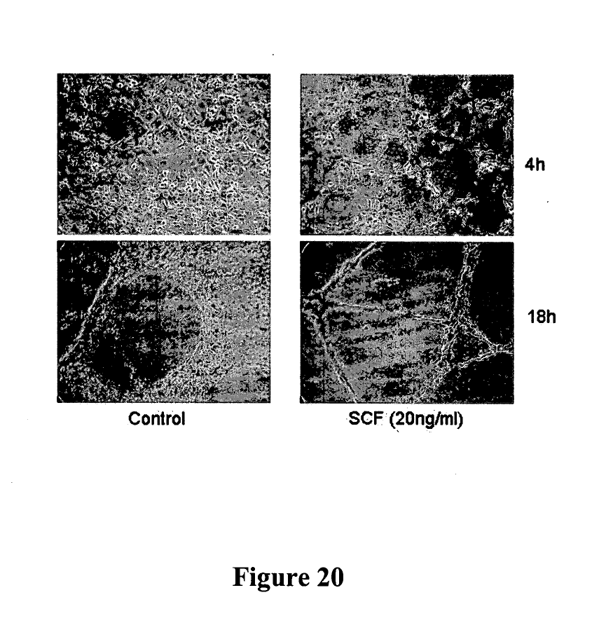

Endothelial cell tube formation on extracellular matrix (ECM) is a commonly used in vitro model of EC differentiation and angiogenesis. We examined the SCF effects on the bMVEC-B cell tube formation. In the presence of the SCF, bMVEC formed tube structures on the matrigel at much earlier time point and more completely compared with SCF-absent control (see FIG. 20).

Conclusion

The current data show that SCF acts not only as the chemokine for the migration of neural stem cells, but also as an angiogenesis factor in the brain. Both functions of SCF are envisioned to be critically important for the development of a novel clinical SCF-based approach to the repair of injured brain tissue.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

What is claimed is:

1. A method of promoting or inhibiting angiogenesis in humans comprising administering to a human in need thereof an effective amount of Stem Cell Factor or a modulator of Stem Cell Factor.

2. The method of claim 1 comprising:
(a) identifying a patient in need of promotion or inhibition of angiogenesis, and
(b) administering to said patient Stem Cell Factor or a modulator of Stem Cell Factor.

3. The method of claim 1 comprising:
(a) administering to a patient Stem Cell Factor or a modulator of Stem Cell Factor, and
(b) measuring in said patient promotion or inhibition of angiogenesis.

4. The method of claim 1 wherein said SCF is administered.

5. A method of promoting or inhibiting angiogenesis in humans comprising administering to a human in need thereof an effective amount of an agonist or antagonist of c-Kit.

6. The method of claim 5 comprising:
(a) identifying a patient in need of promotion or inhibition of angiogenesis, and
(b) administering to said patient an agonist or antagonist of c-Kit.

7. The method of claim 5 comprising:
(a) administering to a patient an agonist or antagonist of c-Kit, and
(b) measuring in said patient promotion or inhibition of angiogenesis.

8. The method of any of claim 5 wherein said antagonist is a small molecule inhibitor.

9. The method of claim 1 wherein said method is to inhibit tumor-induced angiogenesis.

10. The method of claim 5 wherein said method is to inhibit tumor-induced angiogenesis.

11. The method of claim 1 wherein said SCF is defined as the soluble form of the protein of GenBank Accession No. NM_{—}000899 or active variant thereof retaining biological activity and having at least about 65%, 70%, 75%, 80%, 85%, 86%, 87 88%, 89%, 90%, 91%, 92%, 93%, 94%, 95%, 96%, 97%, 98%, or 99% amino acid sequence identity.

12. The method of claim 1 wherein said angiogenesis is within the brain.

13. The method of claim 5 wherein said angiogenesis is within the brain.

14. A method of inducing migration of a neural stem or progenitor cell to a site of neurological injury in the central nervous system of a subject, the method comprising administering Stem Cell Factor or polynucleotide encoding it to a subject in need thereof in an amount effective to stimulate said neural stem or progenitor cell migration.

15. The method of claim 14 wherein said SCF is administered.

16. The method of claim 14 wherein said Stem Cell Factor is defined as the soluble form of the protein of GenBank Accession No. NM_{—}000899 or active variant thereof retaining biological activity and having at least about 65%, 70%, 75%, 80%, 85%, 86%, 87 88%, 89%, 90%, 91%, 92%, 93%, 94%, 95%, 96%, 97%, 98%, or 99% amino acid sequence identity.

17. The method of claim 14 wherein said subject is a human.

18. The method of claim 14 wherein said Stem Cell Factor or polynucleotide encoding it is formulated in a composition for parenteral administration.

19. The method of claim 14 wherein said Stem Cell Factor or polynucleotide encoding it is formulated together with a vehicle in a composition.

20. The method of claim 14 wherein said Stem Cell Factor or polynucleotide encoding it is formulated in unit dosage form.


SEQUENCE LISTING (13 sequences)

SEQ ID NO: 1 - DNA, 28 bp, Artificial Sequence
  Synthetic oligonucleotide
gattgtgtggtttcttcaacattaagtc

SEQ ID NO: 2 - DNA, 15 bp, Artificial Sequence
  Synthetic oligonucleotide
agaaagggaaggcca

SEQ ID NO: 3 - DNA, 24 bp, Artificial Sequence
  Synthetic oligonucleotide
agtataaggctccaaaagcaaagc

SEQ ID NO: 4 - DNA, 20 bp, Artificial Sequence
  Synthetic oligonucleotide
gttgcagccagctcccttag

SEQ ID NO: 5 - DNA, 18 bp, Artificial Sequence
  Synthetic oligonucleotide
cagtagcagtaataggaa

SEQ ID NO: 6 - DNA, 21 bp, Artificial Sequence
  Synthetic oligonucleotide
agaaaacaatgctggcaatgc

SEQ ID NO: 7 - DNA, 20 bp, Artificial Sequence
  Synthetic oligonucleotide
ccatgtggctaaagatgaac

SEQ ID NO: 8 - DNA, 20 bp, Artificial Sequence
  Synthetic oligonucleotide
actgctggtgctcgggtttg

SEQ ID NO: 9 - DNA, 23 bp, Artificial Sequence
  Synthetic oligonucleotide
tatacaaccctggcattatgtcc

SEQ ID NO: 10 - DNA, 20 bp, Artificial Sequence
  Synthetic oligonucleotide
tgcgaaggaggctaaaccta

SEQ ID NO: 11 - DNA, 17 bp, Artificial Sequence
  Synthetic oligonucleotide
actctagcgtgtaaatc

SEQ ID NO: 12 - DNA, 25 bp, Artificial Sequence
  Synthetic oligonucleotide
gaagtcagtcttttcccttgacagt

SEQ ID NO: 13 - DNA, 27 bp, Artificial Sequence
  Synthetic oligonucleotide
gcatgtcacattatactattgcaaaca